SEQUENCE LISTING (26 sequences)

SEQ ID NO: 1 - DNA, 4006 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgaacgcaagtgagttccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggacccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatccgcccatgtgcagcgggcctgggaacacatcaaagagctggcggccgacgtgctgcgagcagagagggagtagaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 2 - DNA, 3093 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 3 - DNA, 2997 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 4 - DNA, 3120 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 5 - DNA, 3390 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgtgcgtgggggctcggcggctgggccgcgggccgtgtgcggctctgctcctcctgggcctggggctaagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 6 - DNA, 3402 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 7 - DNA, 3277 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 8 - DNA, 4775 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcataggtcgacaagcttggtacctccggagcggccgctctagagctagcgacgtcgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 9 - DNA, 3018 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 10 - DNA, 42 bp, Artificial Sequence
  Oligonucleotide UCori-Bam
ggatccagatctactcgaggtagaaaagatcaaaggatcttc

SEQ ID NO: 11 - DNA, 31 bp, Artificial Sequence
  Oligonucleotide UCori-Nco
agtccatggataacgcaggaaagaacatgtg

SEQ ID NO: 12 - DNA, 31 bp, Artificial Sequence
  Oligonucleotide hGH-F
aggatccgaattccctgtgacccctccccag

SEQ ID NO: 13 - DNA, 40 bp, Artificial Sequence
  Oligonucleotide hGH-R
ctctttaccaattctacgcgtaaggacagggaagggagca

SEQ ID NO: 14 - DNA, 40 bp, Artificial Sequence
  Oligonucleotide RO-F
cttccctgtccttacgcgtagaattggtaaagagagtcgt

SEQ ID NO: 15 - DNA, 41 bp, Artificial Sequence
  Oligonucleotide RO-R
ccgtagaaaactagttaatgatttttatcaaaatcattaag

SEQ ID NO: 16 - DNA, 49 bp, Artificial Sequence
  Oligonucleotide RO-1
gaattggtaaagagagtcgtgtaaaatatcgagttcgcacatcttgttg

SEQ ID NO: 17 - DNA, 47 bp, Artificial Sequence
  Oligonucleotide RO-2
gatttttggcgaaaccatttgatcatatgacaagatgtgtatctacc

SEQ ID NO: 18 - DNA, 47 bp, Artificial Sequence
  Oligonucleotide RO-3
atgatttttatcaaaatcattaagttaaggtagatacacatcttgtc

SEQ ID NO: 19 - DNA, 34 bp, Artificial Sequence
  Oligonucleotide Kan-F
aaatcattaactagttttctacggggtctgacgc

SEQ ID NO: 20 - DNA, 33 bp, Artificial Sequence
  Oligonucleotide Kan-R
cagccatggactagtggtggcacttttcgggga

SEQ ID NO: 21 - DNA, 30 bp, Artificial Sequence
  Oligonucleotide MCS1
gatccgatatcgtcgacaagcttggtacct

SEQ ID NO: 22 - DNA, 20 bp, Artificial Sequence
  Oligonucleotide MCS2
caagcttgtcgacgatatcg

SEQ ID NO: 23 - DNA, 32 bp, Artificial Sequence
  Oligonucleotide MCS3
ccggagcggccgctctagagctagcgacgtcg

SEQ ID NO: 24 - DNA, 42 bp, Artificial Sequence
  Oligonucleotide MCS4
aattcgacgtcgctagctctagagcggccgctccggaggtac

SEQ ID NO: 25 - DNA, 25 bp, Artificial Sequence
  Oligonucleotide EF1-Xho
atctcgagcgtgaggctccggtgcc

SEQ ID NO: 26 - DNA, 29 bp, Artificial Sequence
  Oligonucleotide EF1-R
tcggatcctggcttttaggggtagttttc

(19) United States
(12) Patent Application Publication
(10) Pub. No.: US 2026/0041789 A1
(43) Pub. Date: Feb. 12, 2026

(54) Gene therapy DNA vector based on gene therapy DNA vector GDTT1.8NAS12 carrying the therapeutic gene selected from the group of DDC, IL10, IL13, IFNB1, TNFRSF4, TNFSF10, BCL2, HGF, and IL-2 genes for increasing the expression level of these therapeutic genes, method of its production and use, Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.

(71) Applicants: GENETIC DIAGNOSTICS AND THERAPY 21 LTD, London (GB); OBSCHESTVO S OGRANICHENNOI OTVETSTVENNOSTJU "REKOMBITEKH", Moskva (RU)

(72) Inventor: Anton GAMOLSKI, London (GB)

(21) Appl. No.: 18/725,309

(22) PCT Filed: Jan. 15, 2021
(86) PCT No.: PCT/RU2021/000015

§ 371 Date: Oct. 21, 2025

(30) Foreign Application Priority Data
Jan. 15, 2020 (RU) 2020101299

(51) Int. Cl. A61K 48/00; C12N 15/85

(52) U.S. Cl. CPC A61K 48/0058; C12N 15/85; C12N 2800/107

(57) ABSTRACT

Proposed is a gene-therapy DNA vector, based on gene-therapy DNA vector GDTT1.8NAS12, for treating diseases characterized by progressive pathological changes in the nerve tissue structure and neuron function, including neuron death, which are associated with genetic factors, including mutations in genes coding for proteins critical to the normal functioning of the neurons, inter alia Huntington's disease and hereditary forms of amyotrophic lateral sclerosis, as well as with misfolding of the tertiary structure of proteins, inter alia Parkinson's disease and Alzheimer's disease, damage to the central nervous system, disruption of the oxygen supply to the brain or spinal cord, defective neuronal energy metabolism and axonal transport, or autoimmune demyelinating processes, inter alia multiple sclerosis. As a result of the limited size of the vector part GDTT1.8NAS12, which is not greater than 2600 bp, each of the proposed gene-therapy DNA vectors is capable of effectively penetrating into human and animal cells and expressing the target gene cloned within it.

Gene therapy DNA vector based on gene therapy DNA vector GDTT1.8NAS12 carrying the therapeutic gene selected from the group of DDC, IL10, IL13, IFNB1, TNFRSF4, TNFSF10, BCL2, HGF, and IL-2 genes for increasing the expression level of these therapeutic genes, method of its production and use, Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.8NAS12-IFNB1, or Escherichia coli strain JM110-NAS/GDTT1.8NAS12-TNFRSF4, or Escherichia coli strain JM110-NAS/GDTT1.8NAS12-TNFSF10, or Escherichia coli strain JM110-NAS/GDTT1.8NAS12-BCL2, or Escherichia coli strain JM110-NAS/GDTT1.8NAS12-HGF, or Escherichia coli strain JM110-NAS/GDTT1.8NAS12-IL2 carrying the gene therapy DNA vector, method of production thereof, method of gene therapy DNA vector production on an industrial scale

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and subsequent translation of protein molecules encoded by these genes. Within the description of the invention, gene expression refers to the production of a protein molecule with amino acid sequence encoded by this gene. DDC, IL10, IL13, IFNB1, TNFRSF4, TNFSF10, BCL2, HGF, and IL-2 genes included in the group of genes play a key role in several processes in human and animal organisms. A correlation has been shown between low/insufficient concentrations of proteins coded by these genes and different adverse human states and in some cases it is confirmed by disorders in normal expression of genes encoding these proteins. Thus, the increase of the expression of a gene selected from the group of DDC, IL10, IL13, IFNB1, TNFRSF4, TNFSF10, BCL2, HGF, and IL-2 genes through gene therapy has the potential to correct various conditions in humans and animals.

DCC gene (Dopa decarboxylase, Gene ID: 1644) encodes the aromatic L-amino acid decarboxylase (AADC) enzyme catalysing decarboxylation reaction to ensure biosynthesis of dopamine from L-3,4-dihydroxyphenylalanine (L-DOPA), serotonin from L-5-hydroxytryptophan, and tryptamine from L-tryptophan. L-DOPA is obtained from tyrosine in a reaction catalysed by the tyrosine hydroxylase enzyme. Since dopamine does not penetrate brain-blood barrier, L-DOPA is used as a pharmaceutical product for dopamine deficiency compensation in Parkinson's disease patients, thus relieving the symptoms of the Parkinson's disease. L-DOPA provides a strong effect only at the early stages of the Parkinson's disease. The reason is that dopaminergic neurons in striatum, though much fewer in numbers, still contain enough AADC to maintain the required dopamine level (Marsden and Parkes, 1977). However, as the disease progresses, AADC level falls, and L-DOPA application dosage and rate must be increased to achieve the desired clinical effect (Nagatsu et al., 1979). In this regard, gene therapy in the form of AADC encoding gene delivery into striatum is considered as a possible approach to improve clinical response to L-DOPA therapy as Parkinson's disease progresses. E.g., in a MPTP-induced monkey model of Parkinson's disease, a comprehensive therapy was performed, including delivery of human recombinant DCC gene into the striatum using an adeno-associated virus and systemic use of L-DOPA (Bankiewicz et al., 2000, 2006). The results obtained confirmed the therapeutic gene expression for at least 6 years after the introduction and demonstrated the recovery of striatum cells' ability to convert L-DOPA into dopamine. This resulted in achievement of the desired therapeutic effects with significantly smaller dosage of L-DOPA in animals injected with the transgene. Reduced L-DOPA dosage was also decreasing adverse effects of application thereof. In a similar study, positive effects of DCC gene delivery in a monkey model of Parkinson's disease were observed for 15 years (Sehara et al., 2017).

Currently, several research centres are performing clinical studies of gene therapy using a construct encoding the DCC gene and based on the adeno-associated virus serotype 2 (Eberling et al., 2008; Muramatsu et al., 2010; Mittermeyer et al., 2012; Valles et al., 2010).

Interleukin 10 (IL-10) (Gene ID: 3586). Interleukin 10 is an immunomodulating cytokine produced mainly by monocytes and, to a lesser extent, by lymphocytes. It has various effects in immunity and inflammation regulation. In an experimental encephalomyelitis (EEM) model induced by introduction of myelin (MOG1-125), a rat glycoprotein, a construct using the interleukin 10 gene with a point mutation IL-10F129S was introduced intrathecally (Sloane et al., 2008). Analysis of the test and reference animals showed retarded development of paralysis, slower body weight loss, reduced penetration of inflammatory cells into the brain parenchyma, suppressed activation of CNS astrocytes, and suppression of allodynia (increased pain sensitivity to non-painful stimuli). Beside experimental encephalomyelitis, gene therapy involving the gene encoding IL 10 was used in models of chronic pain caused by sciatic nerve ligation (Dengler et al., 2014; Milligan et al., 2006) or taxol injection (Ledeboer et al., 2007). In these studies, delivery was implemented intrathecally using a plasmid vector. Gene therapy effect on chronic pain induction was analysed by a mechanical allodynia test. In all study cases, delivery of the gene encoding IL-10 resulted in normalisation of the mechanical irritation sensitivity thresholds. Suppression of spinal astrocytes and microglia activation by interleukin 10 was believed to constitute the main mechanism of action of such gene therapy in the chronic pain models (Lau et al., 2012).

Interleukin 13 (IL-13) (Gene ID: 3596). Interleukin 13 is an immunomodulating cytokine produced mainly by activated T helper 2 cells. It regulates some maturation and differentiation stages of antibody-producing B-cells, enables antibody production by B-cells to switch to IgE isotype, and suppresses macrophage activity by inhibiting pro-inflammatory cytokine and chemokine synthesis (Mao et al., 2018). As the latest studies imply, IL-13 plays a certain part in pathogenesis of autoimmune diseases including multiple sclerosis (Mao et al., 2018). In a cuprizone-induced demyelination model, the gene encoding IL-13 was delivered by a lentiviral vector into splenium of corpus callosum (Guglielmetti et al., 2016). It resulted in changes in microglia and macrophage response to the demyelinating damage. In particular, IL-13 changes phenotype of microglia and macrophages infiltrating brain tissues, and the microenvironment supporting inflammatory response development. Thus, gene therapy using an IL-13-encoding construct facilitates alleviation of cuprizone-induced demyelination consequences.

Interferon beta 1 (INFB1) (Gene ID: 3456). This gene encodes a cytokine from the family of interferons involved in activation of innate immune response to a pathogen invasion. IFNB1 is crucial for protection mostly against viral infections. In case of multiple sclerosis, recombinant IFNB 1 is used as a first-line drug to relieve the symptoms of chronic inflammation and demyelination in the relapsing form of the disease (Moreno et al., 2010). In a model of experimental encephalomyelitis (EEM) induced by a myelin glycoprotein peptide (MOG35-55) (Hamana et al., 2017), a plasmid vector encoding murine INFB was injected into the tail vein of test animals 7 days after the induction. The construct injected reduced EEM symptoms significantly after one month. Moreover, blood-brain barrier analysis showed that the construct was suppressing the barrier dysfunction specific to EEM. Histological analysis showed that much less inflammatory cells penetrated spinal cord of mice subjected to gene therapy, compared to the reference animals. The authors concluded that a single injection of IFNB-encoding construct suffices for a long-term anti-inflammatory effect in the EEM model.

TNF receptor superfamily member 4 (TNFRSF4, OX40) (Gene ID: 7293) and TNF superfamily member 10 (TNFSF10, TRAIL) (Gene ID: 8743). OX40 gene encodes one of the proteins from the tumour necrosis factor receptor superfamily. Knock-out of this gene suppresses apoptosis in the transformed and tumour cells. The protein encoded by this gene is involved in regulation of B-cell proliferation and differentiation. TRAIL gene encodes a cytokine belonging to a family of apoptosis-inducing ligands related to the tumour necrosis factor. It induces apoptosis in the transformed and tumour cells by binding with its receptors and activating MAPK8/JNK, caspases 8 and 3. In a model of experimental encephalomyelitis (EEM) induced by myeline glycoprotein (MOG) injection (Yellayi et al., 2011), a plasmid within a DNA-lipid complex (lipoplex) was introduced intrathecally (into the cerebellomedullary cistern) on the 8th day after induction (i.e. before manifestation of symptoms). The genetic construct was encoding OX40-TRAIL fusion protein. Genetic construct expression was resulting in synthesis of the fusion protein with immunomodulating properties. Analysis of animals involved assessment of symptom development (tail weakness, weakness and paralysis of hind limbs, etc.) and was performed for 30 days after plasmid injection. Analysis results showed reliable reduction in clinical manifestations of EEM, while histological analysis confirmed expression of the construct in question in the subependymal zone of the 3rd ventricle and significant reduction in the number of inflammatory cells infiltrating the white matter of the spinal cord.

B-Cell Lymphoma protein-2 (BCL2 apoptosis regulator, BCL-2) (Gene ID: 596). BCL-2 gene encodes the protein integrating into the outer mitochondrial membrane and blocking apoptosis in some cell types. It regulates anoxia-induced cell death (Snyder and Chandel, 2009). For example, pCMV-bcl2 or pHRE-bcl2 plasmids within a DNA-liposome complex (lipoplex) were injected intrathecally (into the Cisterna magna) into rats with cerebral ischemia induced by a temporary (for 1.5 hours) ligation of the middle cerebral artery, immediately after induction of the lesion (Cao et al., 2002). BCL-2 protein is localised in the outer mitochondrial membrane where it inhibits various pro-apoptotic factors thus ensuring survival of the cell. In this study, gene therapy using the BCL-2 gene was aimed to increase survival rate of neurons in the brain region affected by ischemia. The study compares vectors with CMV (cytomegaloviral) promoters and HRE (human hypoxia-inducible endothelial growth factor promoter). The transgene is to be expressed throughout the brain in the former case, and predominantly in the ischemia-affected region in the latter. Morphological analysis showed that both constructs produced significant reduction in the number of apoptotic cells, with CMV promoter showing more drastic reduction. And only the CMV promoter demonstrated reliable shrinkage of the infarct zone. The authors concluded on the basis of the results obtained that intrathecal delivery of vectors carrying anti-apoptotic factors has a neuroprotective effect on ischemic lesions.

Hepatocyte growth factor (HGF) (Gene ID: 3082). Hepatocyte growth factor is a plasminogen-dependent pleiotropic growth factor originating in mesenchymal cells (Nakamura et al., 2011). It regulates cell growth, mobility, and morphogenesis of various cell types including epithelial and endothelial cells. In addition, it plays a role in angiogenesis, regeneration of certain organs and tissues, inflammatory reactions, and enhanced growth of nerve cell processes in dorsal root ganglia (Maina and Klein, 1999). It also implements several functions in the central (CNS) and peripheral (PNS) nervous systems, protecting damaged neurons from death and ensuring regeneration of nerve cell processes in CNS and PNS. HGF-encoding plasmid vectors were injected either intramuscularly (Tsuchihara et al., 2009) or intrathecally (Hu et al., 2017) in a model of sciatic nerve or its branch ligation. Mechanosensitivity test results showed recovery of paw withdrawal threshold after the gene therapy performed. As in the case of interleukin 10, suppression of spinal astrocytes and microglia activation was believed to be the main mechanism of HGF action in the chronic pain models.

Interleukin 2 (IL-2) (Gene ID: 3558). Interleukin 2 is a cytokine amplifying T and B cell proliferation after stimulation with an antigen. Beside immunomodulation, IL-2 also acts as a CNS regulatory molecule. IL-2 and its receptor are produced in various brain parts including hippocampus, hypothalamus, cerebellum, neostriatum, and dorsal root ganglia (Hanisch and Quirion, 1995). Accumulated data show that IL-2 plays a major role in nervous and neuroendocrine regulation. Another action of IL-2 in the nervous system is its antinociceptive function both in the peripheral nerves and in the CNS. It is capable of binding to opioid receptors (Wang et al., 1996). In a sciatic nerve ligation model, a plasmid vector encoding IL-2 was injected into rats intrathecally 30 days after chronic pain induction (Yao et al., 2002). Heating sensitivity analysis showed that a single injection of the construct with Lipofectamine resulted in increase of the latent period in response to heating, and that the effect lasted for several days. Blockage of antinociceptive effect of the transgene by naloxone indicates direct action of IL-2 on opioid receptors. The authors concluded that gene therapy involving a gene encoding IL-2 can be used to alleviate neuropathic pain.

Thus, background of the invention suggests that DDC, IL10, IL13, IFNB1, TNFRSF4, TNFSF10, BCL2, HGF, and IL-2 genes have the potential to correct a range of and conditions associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis.

This is why DDC, IL10, IL13, IFNB1, TNFRSF4, TNFSF10, BCL2, HGF, and IL-2 genes are grouped in this patent. Genetic constructs that provide expression of proteins encoded by genes from the group of DDC, IL10, IL13, IFNB1, TNFRSF4, TNFSF10, BCL2, HGF, and IL-2 can be used to develop drugs for the prevention and treatment of various diseases and pathological conditions.

Moreover, these data suggest that insufficient expression of proteins encoded by the DDC, IL10, IL13, IFNB1, TNFRSF4, TNFSF10, BCL2, HGF, and IL-2 genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell, and DNA vectors (Guideline on the quality, non-clinical, and clinical aspects of gene therapy medicinal Products EMA/CAT/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N.//Expert Rev Vaccines. 2016; 15 (3): 313-29).

However, limitations of plasmid vectors use in gene therapy include: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression level of therapeutic genes (promoters, enhancers, post-translational regulatory elements) that constitute nucleotide sequences of genomes of various viruses (Draft Guideline on the quality, non-clinical and clinical aspects of gene therapy medicinal products, http://www.ema.europa.eu/docs/en_GB/document_library/Scientific_guideline/2015/05/WC5001 87020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R.//Mol Biotechnol. 2008.39 (2): 97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HELA cells with 383-4548 bp DNA vectors it was shown that the difference in penetration efficiency can be up to two orders of magnitude (100 times different) (Hornstein B D et al.//PLOS ONE. 2016; 11(12): e0167537.).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2). The vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

Reference is made to the U.S. Pat. No. 10,316,074 describing a method of IL-2 protein production for medical use. The gene encoding IL-2 was included into a recombinant construct for expression in yeast. The disadvantage of this invention is the use of IL-2 protein instead of a gene therapy vector expressing the gene encoding the IL-2 protein.

Reference is made to the U.S. Pat. No. 6,090,791 describing a method to increase the concentration of therapeutic protein, namely IL10, by introduction of a plasmid vector containing a sequence with unmethylated cytosine and guanine nucleotides into the target cells. The disadvantage of this invention is a limited number of cells capable of inducing the target protein production, and vague safety requirements applied to the vector used.

Reference is made to the U.S. Pat. No. 7,598,058 describing methods of modified IL13 protein production, involving synthesis of a polypeptide, DNA sequences encoding that gene, and cloning of those sequences into plasmid vectors. The disadvantage of this invention is the presence of regulatory elements in the DNA vector, that constitute sequences of viral genomes, and presence of antibiotic resistance genes.

The following patents and patent applications are prototypes of this invention with regard to the use of gene therapy approaches to increase the expression level of genes from the group of the DDC, IL10, IL13, IFNB1, TNFRSF4, TNFSF10, BCL2, HGF, and IL-2 genes:

Invention patent U.S. Pat. No. 4,808,523 A describes a plasmid vector carrying the gene encoding IFNB1. This vector enhances the expression of IFNB1 in mammalian cells. The disadvantage of this invention is a method of use limited to the production of IFNB 1 protein in vitro and different from the gene therapy use of this vector. Also, the disadvantage of this invention is the presence of sequences of viral origin in the vector.

Invention patent RU 2678756 describes gene therapy DNA vector VTvaf17 and method of production thereof; Escherichia coli strain SCS110-AF and method of production thereof; Escherichia coli strain SCS110-AF/VTvaf17 carrying the gene therapy DNA vector VTvaf17 and method of production thereof. The disadvantage of this invention is the considerable length of the vector part, which may have an adverse effect on the efficiency of DNA vector delivery to cells.

Invention patent U.S. Pat. No. 8,647,618 describing a delivery method for a gene therapy vector carrying an IL-2 gene sequence in an attenuated Salmonella typhimurium strain for treatment of oncology diseases. A disadvantage of this method is the induction of an immune response to repeated introduction of bacterial cells and, therefore, a critical reduction of the therapeutic effect and vague requirements to safety of the vector used.

Invention patent U.S. Pat. No. 8,389,492 describing an expressing plasmid vector that contains a gene encoding hepatocyte growth factor (HGF) for treatment of ischemic and hepatic diseases. The patent describes two routes of vector introduction, as a naked DNA, and within a liposome complex. The disadvantage of this invention is the presence of kanamycin antibiotic resistance genes in the vector.

### SUMMARY OF THE INVENTION

The purpose of this invention is to construct the gene therapy DNA vectors increasing expression level of a group of DDC, IL10, IL13, IFNB1, TNFRSF4, TNFSF10, BCL2, HGF, and IL-2 genes in humans and animals that combine the following properties:

I) Efficiency of the gene therapy DNA vector in increasing the expression level of therapeutic genes in eukaryotic cells.

II) Possibility of safe use in gene therapy of human beings and animals due to the absence of regulatory elements representing the nucleotide sequences of viral genomes in the gene therapy DNA vector.

III) Possibility of safe use in the gene therapy of human beings and animals due to the absence of antibiotic resistance genes in the gene therapy DNA vector.

IV) Producibility and constructability of the gene therapy DNA vector on an industrial scale.

Items II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by constructing gene therapy DNA vector based on the gene therapy DNA vector GDTT1.8NAS12 in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis, while the gene therapy DNA vector GDTT1.8NAS12-DDC contains the coding region of the DDC therapeutic gene cloned into the gene therapy DNA vector GDTT1.8NAS12, with the nucleotide sequence SEQ ID No. 1, the gene therapy DNA vector GDTT1.8NAS12-IL10 contains the coding region of the IL 10 therapeutic gene cloned into the gene therapy DNA vector GDTT1.8NAS12, with the nucleotide sequence SEQ ID No. 2, the gene therapy DNA vector GDTT1.8NAS12-IL13 contains the coding region of the IL13 therapeutic gene cloned into the gene therapy DNA vector GDTT1.8NAS12, with the nucleotide sequence SEQ ID No. 3, the gene therapy DNA vector GDTT1.8NAS12-IFNB1 contains the coding region of the IFNB1 therapeutic gene cloned into the gene therapy DNA vector GDTT1.8NAS12, with the nucleotide sequence SEQ ID No. 4, the gene therapy DNA vector GDTT1.8NAS12-TNFRSF4 contains the coding region of the TNFRSF4 therapeutic gene cloned into the gene therapy DNA vector GDTT1.8NAS12, with the nucleotide sequence SEQ ID No. 5, the gene therapy DNA vector GDTT1.8NAS12-TNFSF10 contains the coding region of the TNFSF10 therapeutic gene cloned into the gene therapy DNA vector GDTT1.8NAS12, with the nucleotide sequence SEQ ID No. 6, the gene therapy DNA vector GDTT1.8NAS12-BCL2 contains the coding region of the BCL2 therapeutic gene cloned into the gene therapy DNA vector GDTT1.8NAS12, with the nucleotide sequence SEQ ID No. 7, the gene therapy DNA vector GDTT1.8NAS12-HGF contains the coding region of the HGF therapeutic gene cloned into the gene therapy DNA vector GDTT1.8NAS12, with the nucleotide sequence SEQ ID No. 8, the gene therapy DNA vector GDTT1.8NAS12-IL2 contains the coding region of the IL-2 therapeutic gene cloned into the gene therapy DNA vector GDTT1.8NAS12, with the nucleotide sequence SEQ ID No. 9.

Each of the constructed gene therapy DNA vectors, namely GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, has the ability to efficiently penetrate into human and animal cells and express the DDC, IL10, IL13, IFNB1, TNFRSF4, TNFSF10, BCL2, HGF, or IL-2 therapeutic gene cloned into it due to the limited size of GDTT1.8NAS12 vector part not exceeding 2600 bp.

Each of the constructed gene therapy DNA vectors, namely: GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, uses nucleotide sequences that don't constitute antibiotic resistance genes, viral genes, or regulatory elements of viral genomes, which ensures its safe use for gene therapy in humans and animals.

A method of gene therapy DNA vector production based on the gene therapy DNA vector GDTT1.8NAS12 carrying the therapeutic gene DDC, or IL10, or IL13, or IFNB1, or TNFRSF4, or TNFSF10, or BCL2, or HGF, or IL-2 was also developed, that involves obtaining each of gene therapy DNA vectors: GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 as follows: the coding region of the therapeutic gene from the group of DDC, or IL10, or IL13, or IFNB1, or TNFRSF4, or TNFSF10, or BCL2, or HGF, or IL-2 gene is cloned to DNA vector GDTT1.8NAS12, and the gene therapy DNA vector GDTT1.8NAS12-DDC, SEQ ID No. 1, or GDTT1.8NAS12-IL10, SEQ ID No. 2, or GDTT1.8NAS12-IL13, SEQ ID No. 3, or GDTT1.8NAS12-IFNB1, SEQ ID No. 4, or GDTT1.8NAS12-TNFRSF4, SEQ ID No. 5, or GDTT1.8NAS12-TNFSF10, SEQ ID No. 6, or GDTT1.8NAS12-BCL2, SEQ ID No. 7, or GDTT1.8NAS12-HGF, SEQ ID No. 8, or GDTT1.8NAS12-IL2, SEQ ID No. 9, respectively, is obtained, while the coding region of the DDC, or IL10, or IL13, or IFNB1, or TNFRSF4, or TNFSF10, or BCL2, or HGF, or IL-2 therapeutic gene is obtained by isolating total RNA from a biological human tissue sample, followed by reverse transcription and PCR amplification using the obtained oligonucleotides and cleaving the amplification product by corresponding restriction endonucleases, while cloning to the gene therapy DNA vector GDTT1.8NAS12 is performed by SalI and HindIII, or SalI and KpnI, or BamHI and SalI restriction sites, while the selection is performed without antibiotics, the following oligonucleotides are used as dedicated oligonucleotides for reverse transcription and PCR amplification in the process of production of the gene therapy DNA vector GDTT1.8NAS12-DDC, SEQ ID No. 1:

DDC-F
ATCGTCGACCACCATGAACGCAAGTGAGTTCCGA,
DDC-R
ACCAAGCTTCTACTCCCTCTCTG,

and the cleaving of the amplification product and cloning of the coding region of the DDC gene into the gene therapy DNA vector GDTT1.8NAS12 is performed using SalI and HindIII restriction endonucleases,
   the following oligonucleotides are used as dedicated oligonucleotides for reverse transcription and PCR amplification in the process of production of the gene therapy DNA vector GDTT1.8NAS12-IL10, SEQ ID No. 2:

IL10-F
AGGATCCACCATGCACAGCTCAGCACTGC,
IL10-R
CTTGTCGACTCAGTTTCGTATCTTCATTGTC,

and the cleaving of the amplification product and cloning of the coding region of the IL10 gene into the gene therapy DNA vector GDTT1.8NAS12 is performed using BamHI and SalI restriction endonucleases,
   the following oligonucleotides are used as dedicated oligonucleotides for reverse transcription and PCR amplification in the process of production of the gene therapy DNA vector GDTT1.8NAS12-IL13, SEQ ID No. 3:

IL13-F
AGGATCCACCATGCATCCGCTCCTCAATC,
IL13-R
CTTGTCGACTCAGTTGAACTGTCCCTCG,

and the cleaving of the amplification product and cloning of the coding region of the IL13 gene into the gene therapy DNA vector GDTT1.8NAS12 is performed using BamHI and SalI restriction endonucleases,
the following oligonucleotides are used as dedicated oligonucleotides for reverse transcription and PCR amplification in the process of production of the gene therapy DNA vector GDTT1.8NAS12-IFNB1, SEQ ID No. 4:

IFNB1-F
AAAGGATCCACCATGACCAACAAGTGTCTCCTCCAAA,
IFNB1-R
TTTGTCGACTCAGTTTCGGAGGTAACCTGTAAGTCTG,

and the cleaving of the amplification product and cloning of the coding region of the IFNB1 gene into the gene therapy DNA vector GDTT1.8NAS12 is performed using BamHI and SalI restriction endonucleases,
the following oligonucleotides are used as dedicated oligonucleotides for reverse transcription and PCR amplification in the process of production of the gene therapy DNA vector GDTT1.8NAS12-TNFRSF4, SEQ ID No. 5:

TNFRSF4-F
AGGATCCACCATGTGCGTGGGGGCTCGG,
TNFRSF4-R
CTTGTCGACTCAGATCTTGGCCAGGGTG,

and the cleaving of the amplification product and cloning of the coding region of the TNFRSF4 gene into the gene therapy DNA vector GDTT1.8NAS12 is performed using BamHI and SalI restriction endonucleases,
the following oligonucleotides are used as dedicated oligonucleotides for reverse transcription and PCR amplification in the process of production of the gene therapy DNA vector GDTT1.8NAS12-TNFSF10, SEQ ID No. 6:

TNFSF10-F
AGGATCCACCATGGCTATGATGGAGGTCCA,
TNFSF10-R
TGTCGACTCAGCCAACTAAAAAGGCCCCGA,

and the cleaving of the amplification product and cloning of the coding region of the TNFSF10 gene into the gene therapy DNA vector GDTT1.8NAS12 is performed using BamHI and SalI restriction endonucleases,
the following oligonucleotides are used as dedicated oligonucleotides for reverse transcription and PCR amplification in the process of production of the gene therapy DNA vector GDTT1.8NAS12-BCL2, SEQ ID No. 7:

BCL-F
ATCGTCGACCACCATGGCGCACGCTGGGAGA,
BCL-R
TTCGGTACCTCACTTGTGGCCCA,

and the cleaving of the amplification product and cloning of the coding region of the BCL2 gene into the gene therapy DNA vector GDTT1.8NAS12 is performed using SalI and KpnI restriction endonucleases,
the following oligonucleotides are used as dedicated oligonucleotides for reverse transcription and PCR amplification in the process of production of the gene therapy DNA vector GDTT1.8NAS12-HGF, SEQ ID No. 8:

HGF_F
TTTGGATCCACCATGTGGGTGACCAAACTCCTGCCA,
HGF_R
AATGTCGACCTATGACTGTGGTACCTTATATGTTAAAAT,

and the cleaving of the amplification product and cloning of the coding region of the HGF gene into the gene therapy DNA vector GDTT1.8NAS12 is performed using BamHI and SalI restriction endonucleases,
the following oligonucleotides are used as dedicated oligonucleotides for reverse transcription and PCR amplification in the process of production of the gene therapy DNA vector GDTT1.8NAS12-IL2, SEQ ID No. 9:

IL-2-F
AGGATCCACCATGTACAGGATGCAACTCCT,
IL-2-R
TGTCGACTCAAGTCAGTGTTGAGATGATG,

and the cleaving of the amplification product and cloning of the coding region of the IL-2 gene into the gene therapy DNA vector GDTT1.8NAS12 is performed using BamHI and SalI restriction endonucleases.

A method of use of the gene therapy DNA vector based on gene therapy DNA vector GDTT1.8NAS12 carrying DDC, IL10, IL13, IFNB1, TNFRSF4, TNFSF10, BCL2, HGF, or IL-2 therapeutic gene in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis was developed that involves transfection of the cells of patient or animal organs and tissues with the selected gene therapy DNA vector carrying the therapeutic gene based on the gene therapy DNA vector GDTT1.8NAS12 or several selected gene therapy DNA vectors carrying the therapeutic genes based on the gene therapy DNA vector GDTT1.8NAS12 from the group of constructed gene therapy DNA vectors carrying the therapeutic genes based on the gene therapy DNA vector GDTT1.8NAS12 and/or injection of autologous cells of a patient or animal transfected with the selected gene therapy DNA vector carrying the therapeutic gene based on the gene therapy DNA vector GDTT1.8NAS12 or several gene therapy DNA vectors carrying the therapeutic genes based on the gene therapy DNA vector GDTT1.8NAS12 selected from a group of the constructed gene therapy DNA vectors carrying the therapeutic genes based on the gene therapy DNA vector GDTT1.8NAS12 into cells of patient or the same animal organs and tissues, and/or injection of the gene therapy DNA vector carrying the therapeutic gene based on the gene therapy DNA vector GDTT1.8NAS12 or several selected gene therapy DNA vectors carrying the therapeutic genes based on the gene therapy DNA vector GDTT1.8NAS12, selected from the group of constructed gene therapy DNA vectors carrying therapeutic genes based on the gene therapy DNA vector GDTT1.8NAS12 into the organs and tissues of the same patient or animal, or the combination of these methods.

A method of production of strain for construction of the gene therapy DNA vector in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis was developed that involves obtaining electrocompetent cells of Escherichia coli strain JM110-NAS and subjecting these cells to electroporation with the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2. After that, the cells are seeded to agar plates with a selective medium containing yeastrel, peptone, 6% sucrose, and 10 μg/ml of chloramphenicol, and as a result, Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.8NAS12-IFNB1, or Escherichia coli strain JM110-NAS/GDTT1.8NAS12-TNFRSF4, or Escherichia coli strain JM110-NAS/GDTT1.8NAS12-TNFSF10, or Escherichia coli strain JM110-NAS/GDTT1.8NAS12-BCL2, or Escherichia coli strain JM110-NAS/GDTT1.8NAS12-HGF, or Escherichia coli strain JM110-NAS/GDTT1.8NAS12-IL2 is obtained.

Escherichia coli strain JM110-NAS/GDTT1.8NAS12-DDC carrying the gene therapy DNA vector GDTT1.8NAS12-DDC for its production allowing for antibiotic-free selection in the process of the gene therapy DNA vector production, or Escherichia coli strain JM110-NAS/GDTT1.8NAS12-IL 10 carrying the gene therapy DNA vector GDTT1.8NAS12-IL10 for its production allowing for antibiotic-free selection in the process of the gene therapy DNA vector production, or Escherichia coli strain JM110-NAS/GDTT1.8NAS12-IL13 carrying the gene therapy DNA vector GDTT1.8NAS12-IL13 for its production allowing for antibiotic-free selection in the process of the gene therapy DNA vector production, or Escherichia coli strain JM110-NAS/GDTT1.8NAS12-IFNB1 carrying the gene therapy DNA vector GDTT1.8NAS12-IFNB 1 for its production allowing for antibiotic-free selection in the process of the gene therapy DNA vector production, or Escherichia coli strain JM110-NAS/GDTT1.8NAS12-TNFRSF4 carrying the gene therapy DNA vector GDTT1.8NAS12-TNFRSF4 for its production allowing for antibiotic-free selection in the process of the gene therapy DNA vector production, or Escherichia coli strain JM110-NAS/GDTT1.8NAS12-TNFSF10 carrying the gene therapy DNA vector GDTT1.8NAS12-TNFSF10 for its production allowing for antibiotic-free selection in the process of the gene therapy DNA vector production, or Escherichia coli strain JM110-NAS/GDTT1.8NAS12-BCL2 carrying the gene therapy DNA vector GDTT1.8NAS12-BCL2 for its production allowing for antibiotic-free selection in the process of the gene therapy DNA vector production, or Escherichia coli strain JM110-NAS/GDTT1.8NAS12-HGF carrying the gene therapy DNA vector GDTT1.8NAS12-HGF for its production allowing for antibiotic-free selection in the process of the gene therapy DNA vector production, or Escherichia coli strain JM110-NAS/GDTT1.8NAS12-IL2 carrying the gene therapy DNA vector GDTT1.8NAS12-IL2 for its production allowing for antibiotic-free selection in the process of the gene therapy DNA vector production is claimed in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis.

A method of industrial-scale production of the gene therapy DNA vector based on the gene therapy DNA vector GDTT1.8NAS12 carrying the DDC, or IL10, or IL13, or IFNB1, or TNFRSF4, or TNFSF10, or BCL2, or HGF, or IL-2 therapeutic gene in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis was developed that involves production of the gene therapy DNA vector GDTT1.8NAS12-DDC, or the gene therapy DNA vector GDTT1.8NAS12-IL10, or the gene therapy DNA vector GDTT1.8NAS12-IL13, or the gene therapy DNA vector GDTT1.8NAS12-IFNB1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2 by inoculating a culture flask containing the prepared medium with seed culture selected from the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2, then the cell culture is incubated in an incubator shaker and transferred to an industrial fermenter, then grown to a stationary phase, then the fraction containing the therapeutic DNA product is extracted, multi-stage filtered, and purified by chromatographic methods.

### BRIEF DESCRIPTION OF DRAWINGS

FIG. 1

shows the structure of the gene therapy DNA vector GDTT1.8NAS12 carrying the therapeutic gene selected from the group of DDC, IL10, IL13, IFNB1, TNFRSF4, TNFSF10, BCL2, HGF, and IL-2 genes that constitutes a circular double-stranded DNA molecule capable of autonomous replication in Escherichia coli cells.

FIG. 1 shows the structures corresponding to:

FIG. 1A is the gene therapy DNA vector GDTT1.8NAS12-DDC,

FIG. 1B is the gene therapy DNA vector GDTT1.8NAS12-IL10,

FIG. 1C is the gene therapy DNA vector GDTT1.8NAS12-IL13,

FIG. 1D is the gene therapy DNA vector GDTT1.8NAS12-IFNB1,

FIG. 1E is the gene therapy DNA vector GDTT1.8NAS12-TNFRSF4,

FIG. 1F is the gene therapy DNA vector GDTT1.8NAS12-TNFSF10,

FIG. 1G is the gene therapy DNA vector GDTT1.8NAS12-BCL2,

FIG. 1H is the gene therapy DNA vector GDTT1.8NAS12-HGF,

FIG. 1I is the gene therapy DNA vector GDTT1.8NAS12-IL2.

The following structural elements of the vector are indicated in the structures:
EF1a pr—the promoter region of human elongation factor EF1A with an intrinsic enhancer contained in the first intron of the gene. It ensures efficient transcription of the recombinant gene in most human tissues;

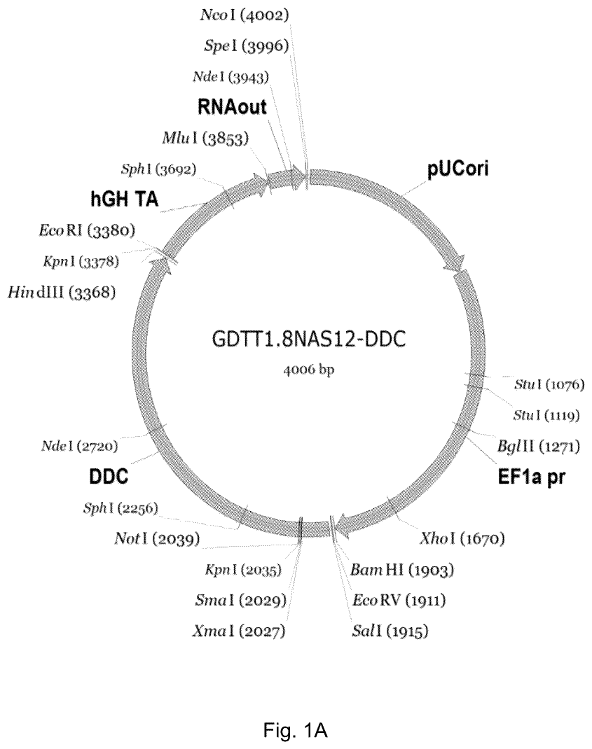

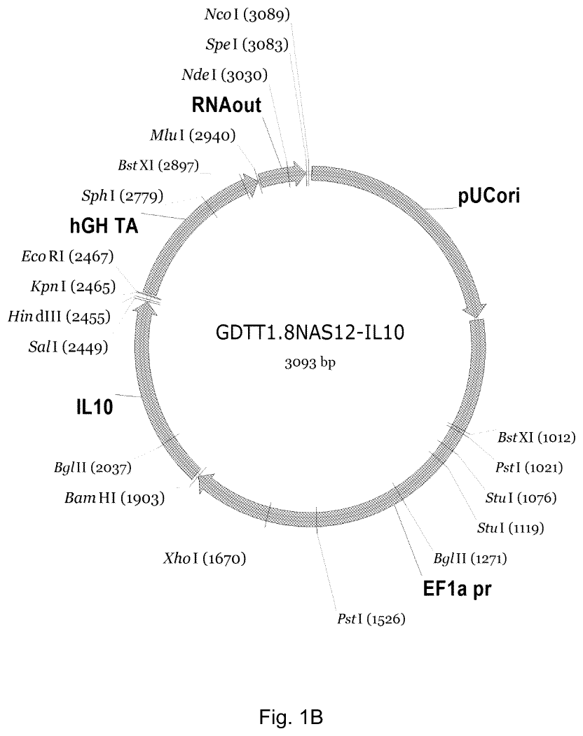

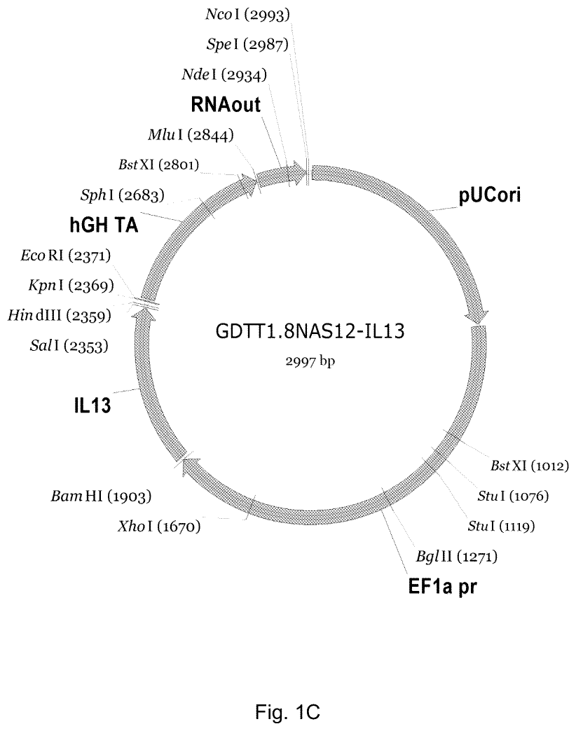

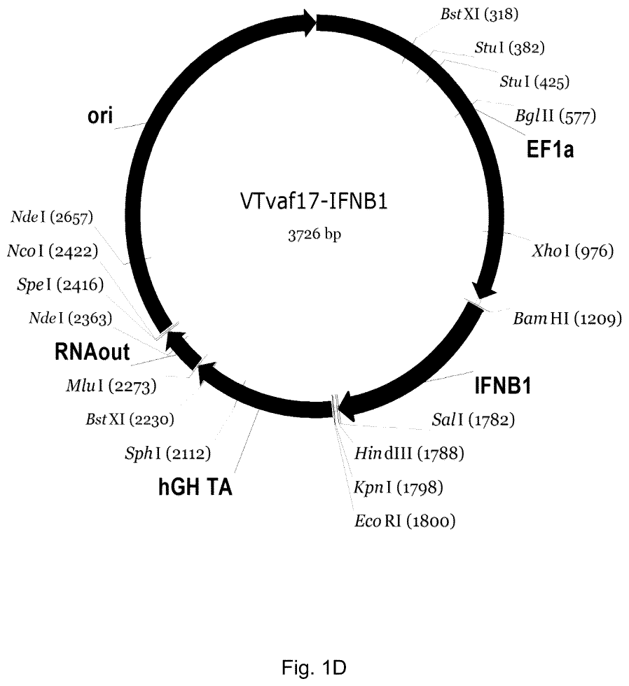

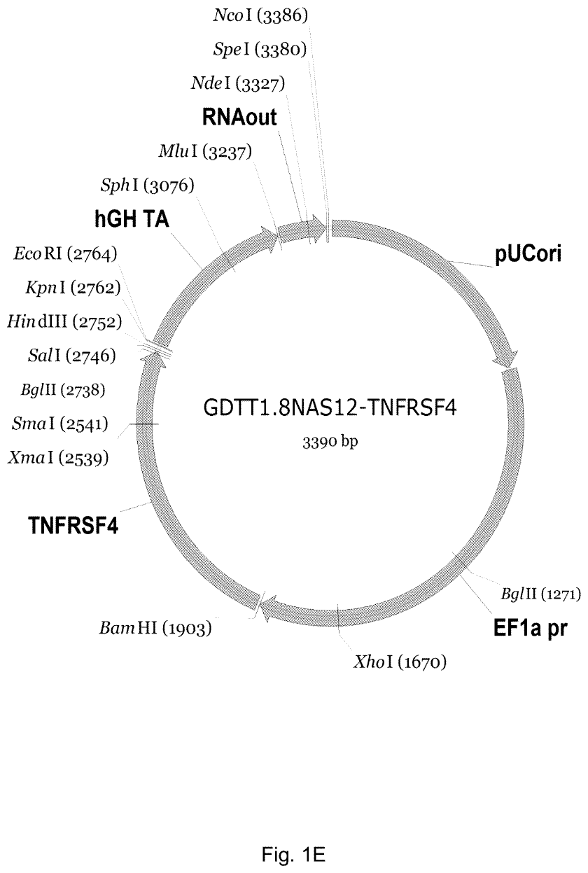

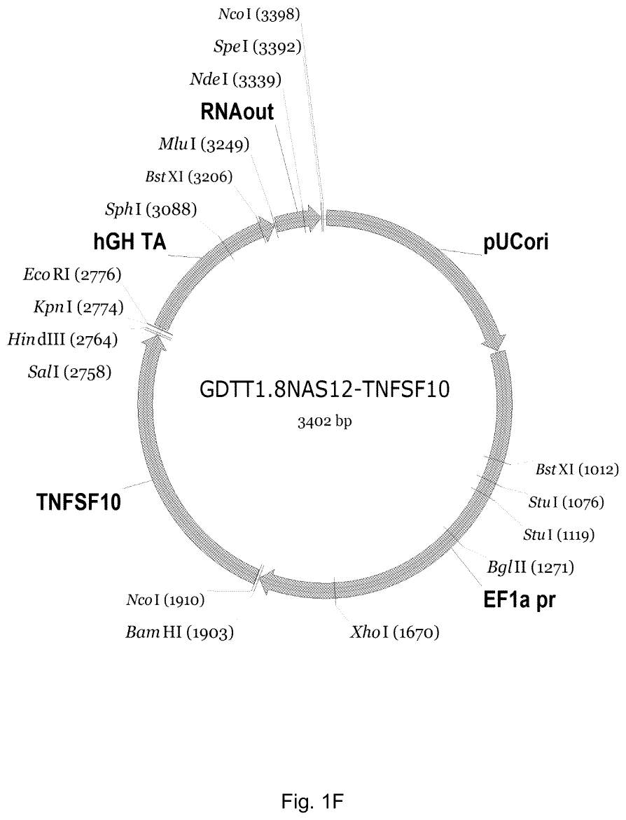

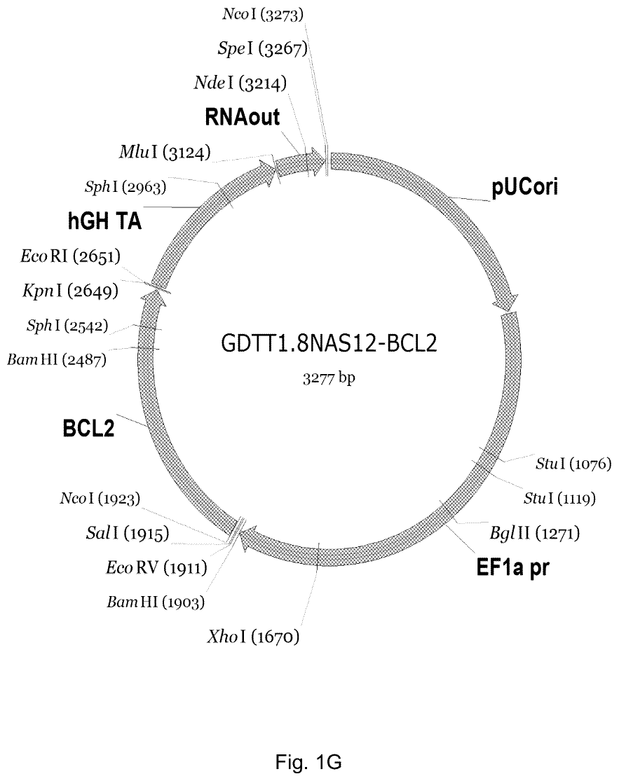

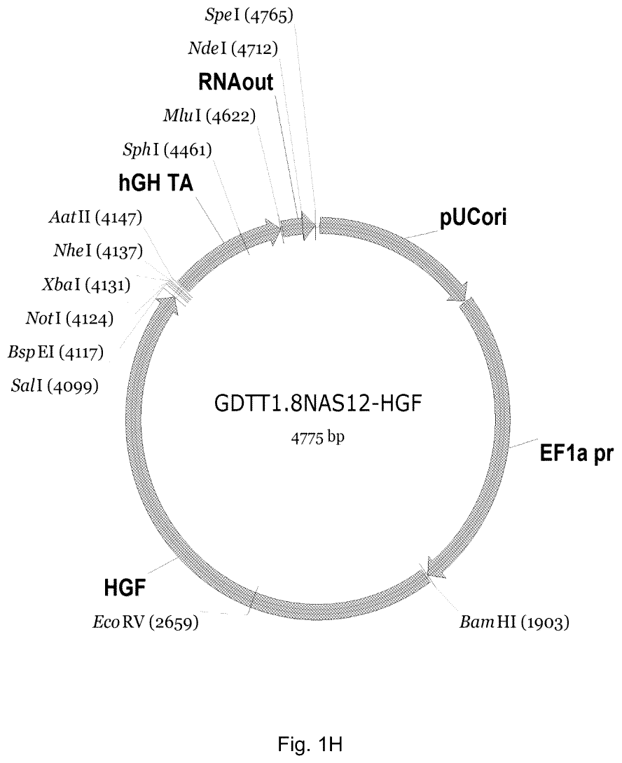

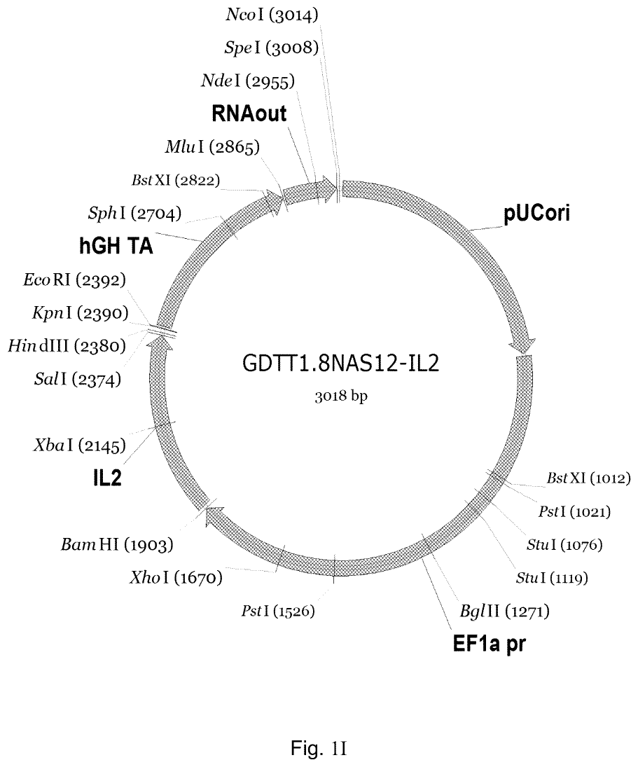

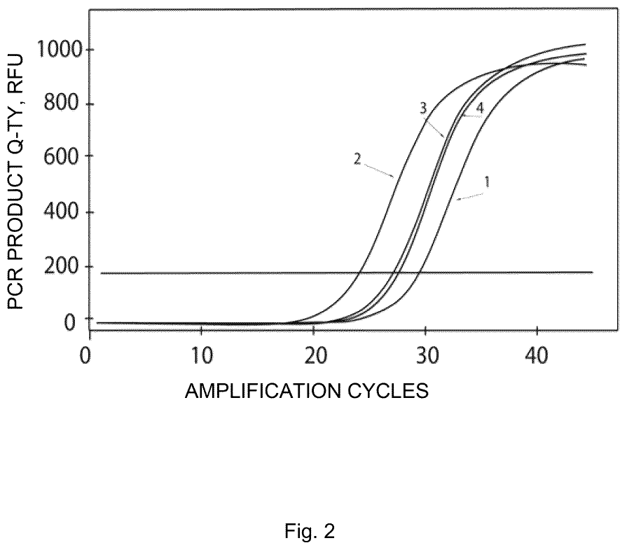

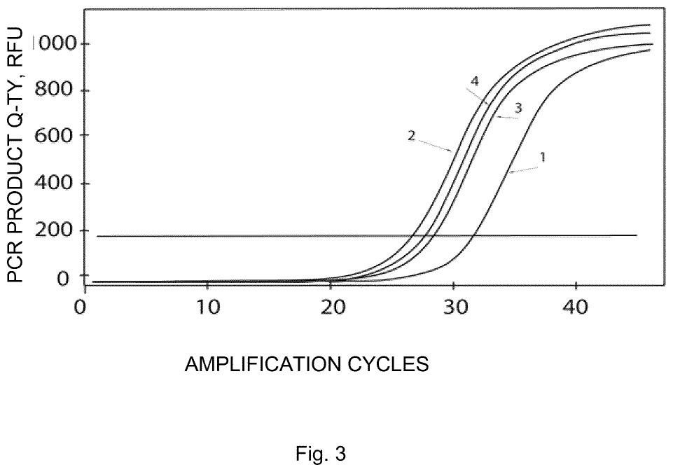

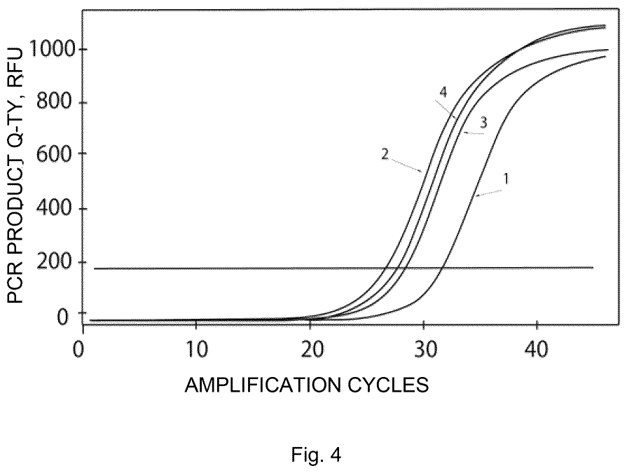

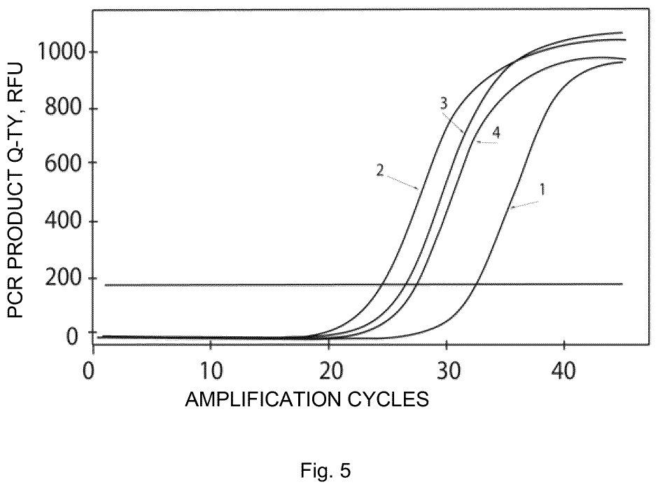

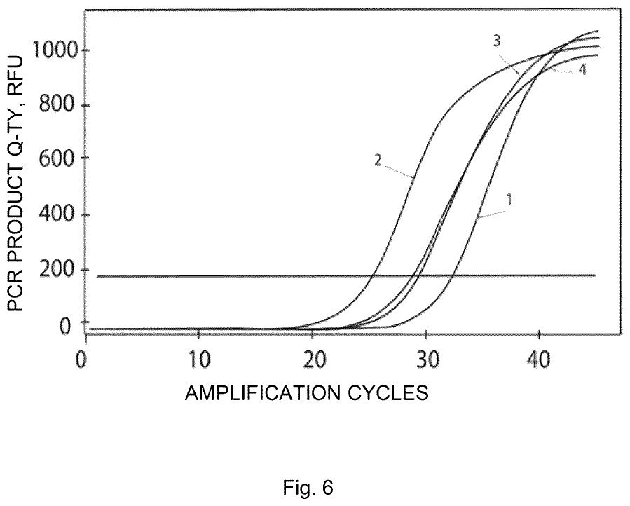

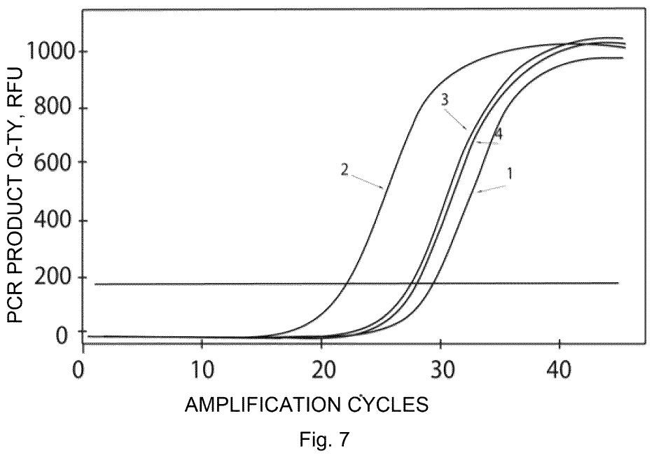

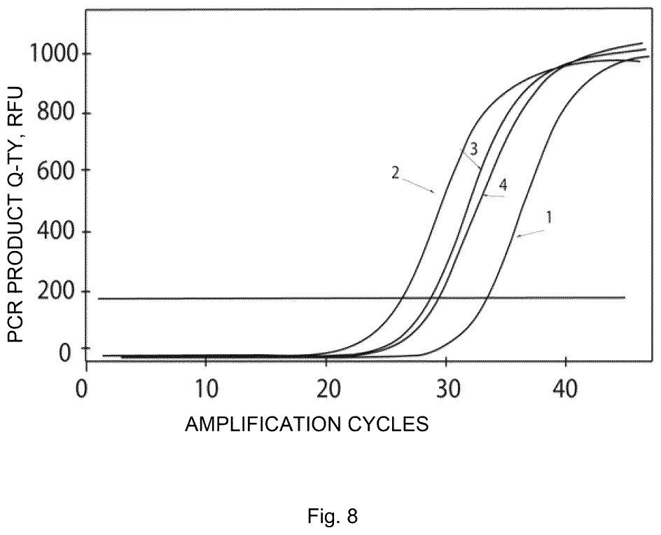

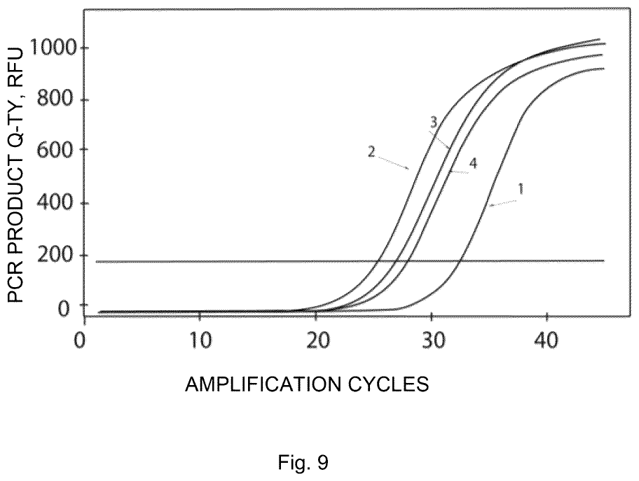

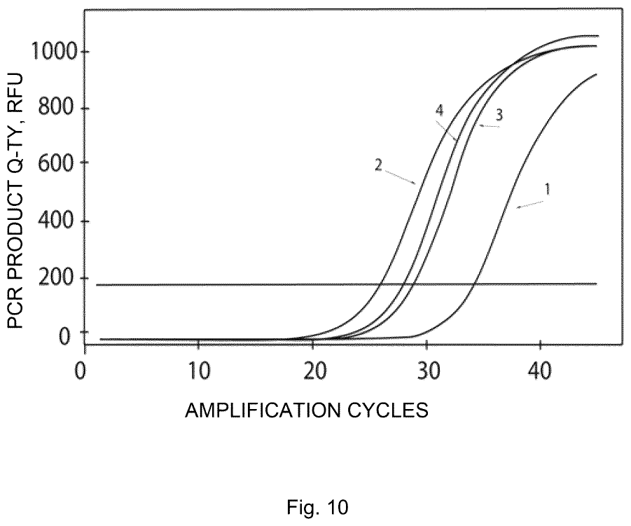

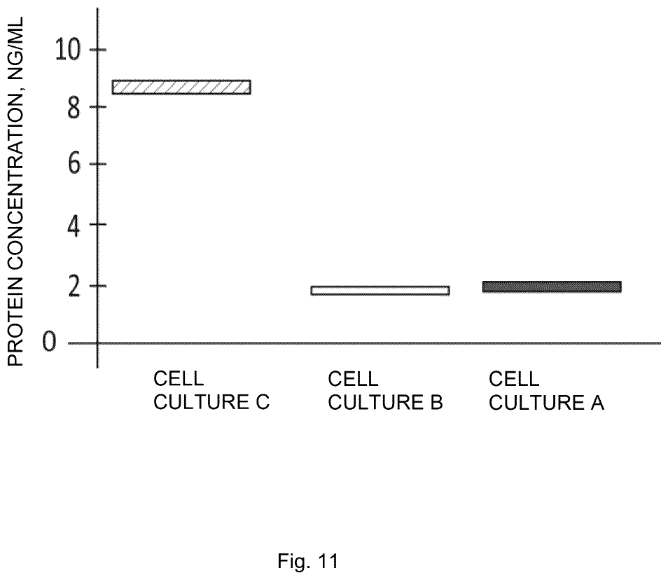

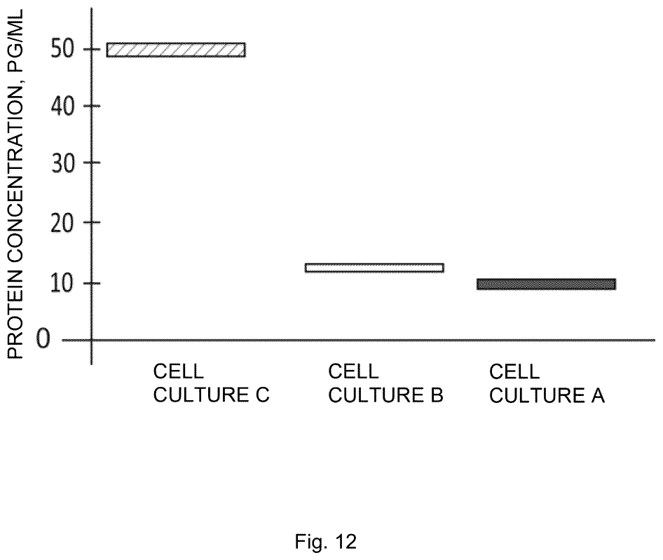

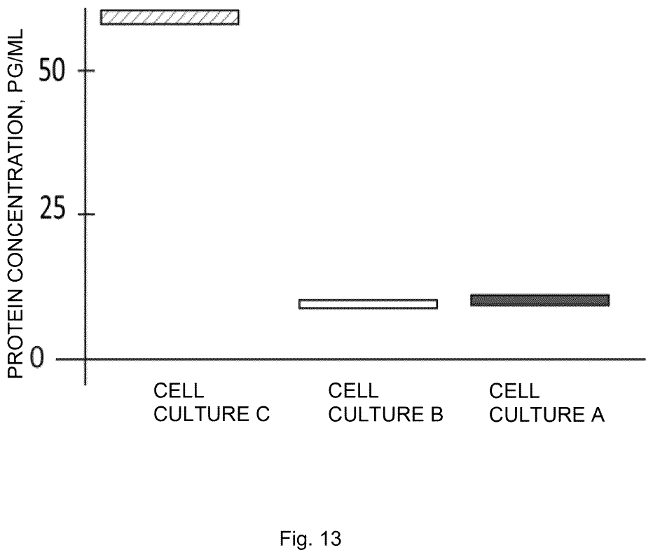

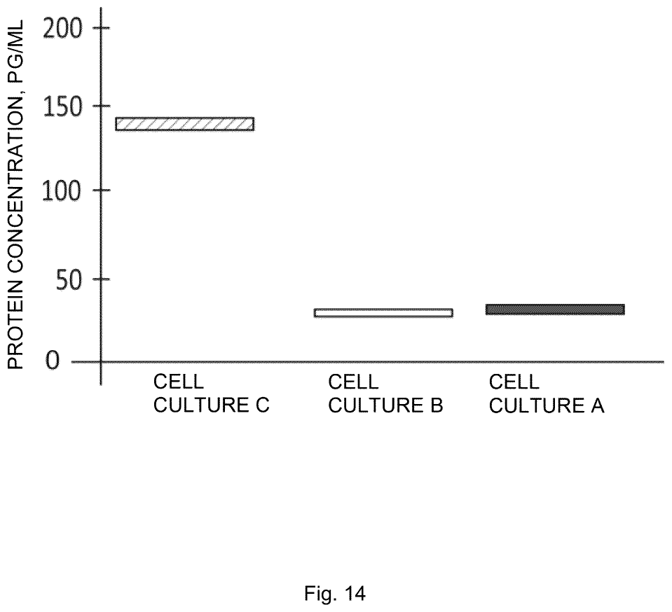

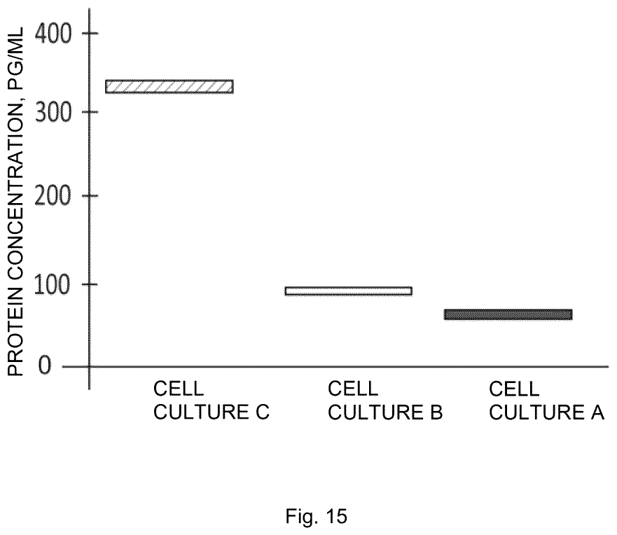

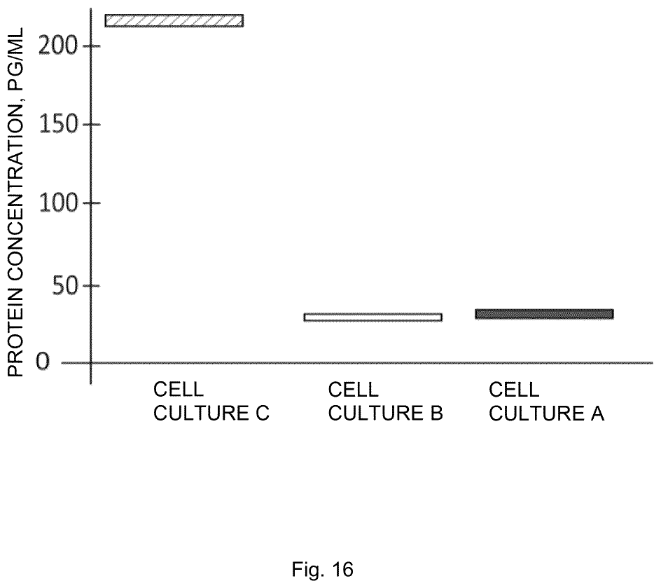

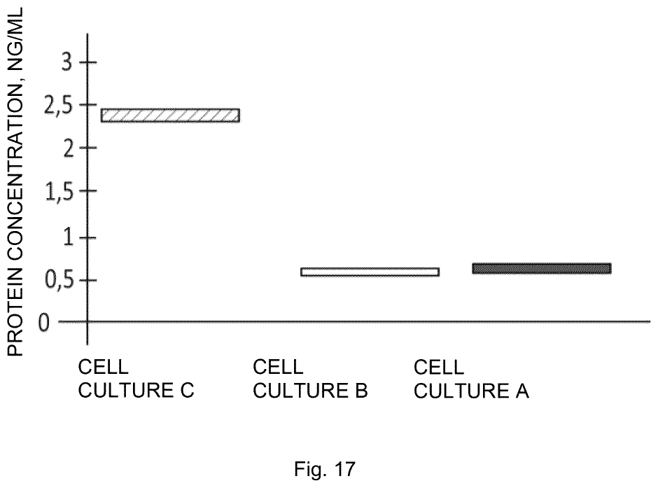

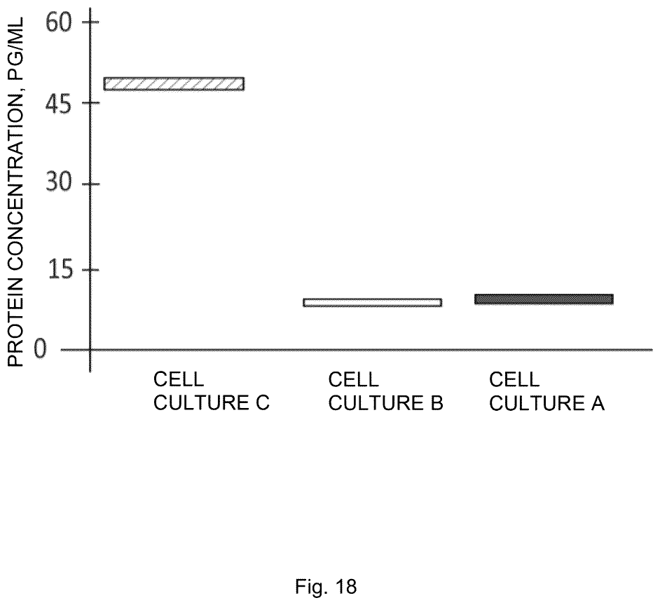

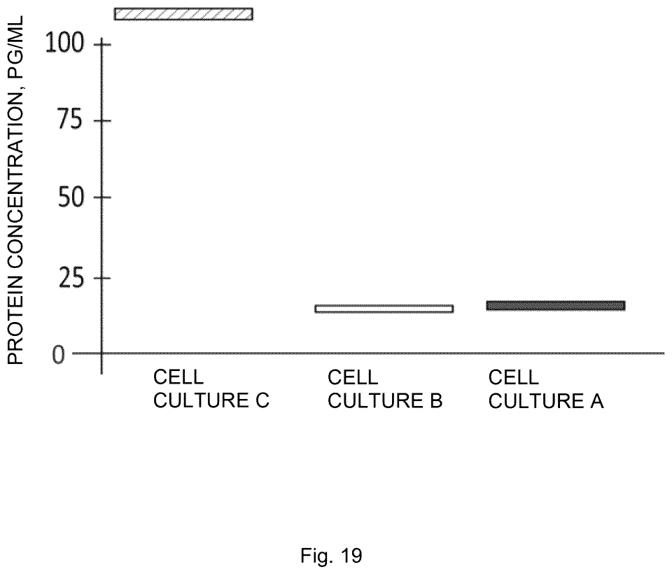

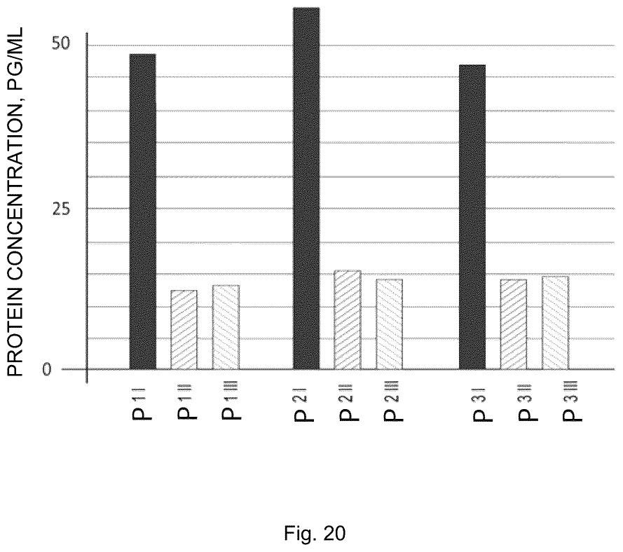

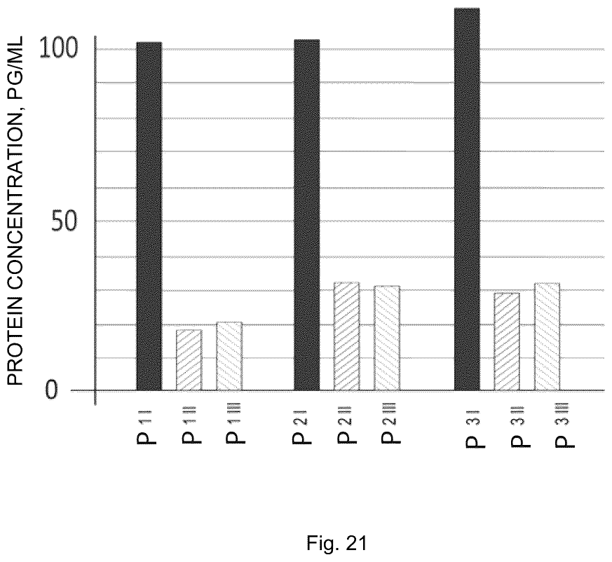

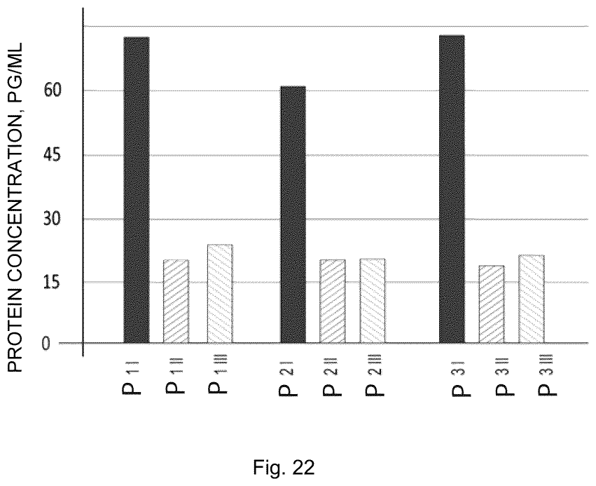

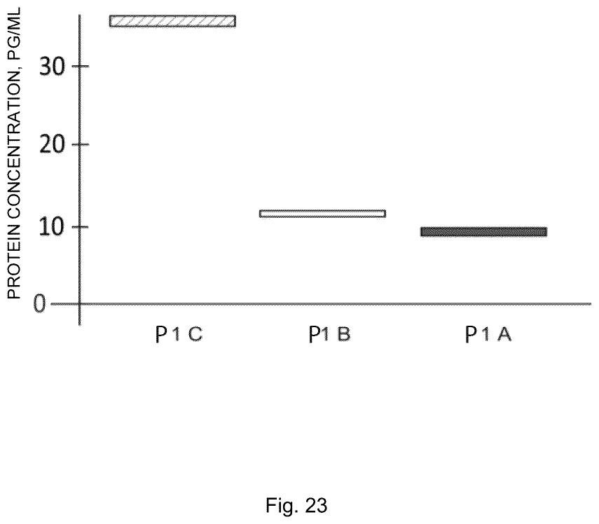

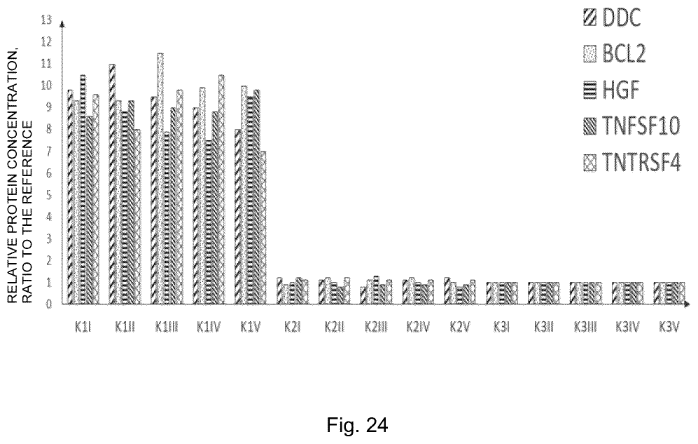

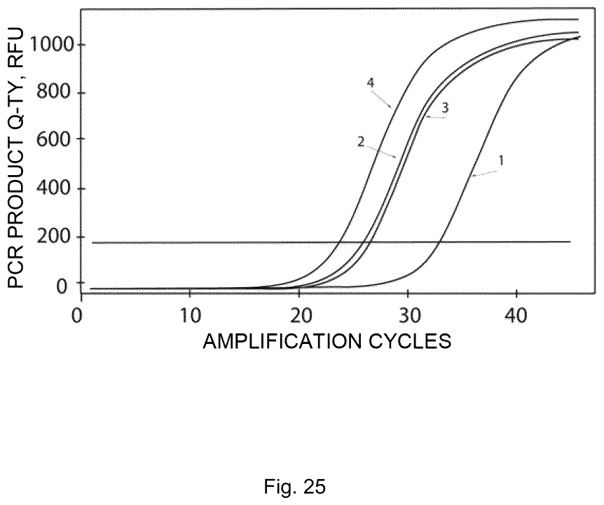

The reading frame of the therapeutic gene corresponding to the coding region of the DDC gene (FIG. 1A), or the IL10 gene (FIG. 1B), or the IL13 gene (FIG. 1C), or the IFNB1 gene (FIG. 1D), or the TNFRSF4 gene (FIG. 1E), or the TNFSF10 gene (FIG. 1F), or the BCL2 gene (FIG. 1G), or the HGF gene (FIG. 1H), or the IL-2 gene (FIG. 1I), respectively;
hGH TA—transcription terminator;
pUCori—the origin of replication for autonomous replication with a single nucleotide substitution to increase plasmid production in the cells of most Escherichia coli strains;
RNA-out—the regulatory element RNA-out of transposon In 10 that allows for antibiotic-free positive selection in case of the use of Escherichia coli strain JM110-NAS.

Unique restriction sites are marked.

FIG. 2

shows diagrams of cDNA amplicon accumulation of the therapeutic gene, namely the DDC gene, in the HCN-2 human cortical neuron cell line (ATCC CRL-10742) before its transfection and 48 hours after transfection with the gene therapy DNA vector GDTT1.8NAS12-DDC in order to assess the ability to penetrate into eukaryotic cells and functional activity, i.e. expression of the therapeutic gene at the mRNA level.

FIG. 2 shows curves of accumulation of amplicons during the reaction corresponding to:
1—cDNA of the DDC gene in the HCN-2 cell line before transfection with the DNA vector GDTT1.8NAS12-DDC,
2—cDNA of the DDC gene in the HCN-2 cell line after transfection with DNA vector GDTT1.8NAS12-DDC,
3—cDNA of the B2M gene in the HCN-2 cell line before transfection with the DNA vector GDTT1.8NAS12-DDC,
4—cDNA of the B2M gene in the HCN-2 cell line after transfection with the DNA vector GDTT1.8NAS12-DDC.

The B2M (beta-2-microglobulin) gene listed in the GenBank database under number NM 004048.2 was used as a reference gene.

FIG. 3

shows diagrams of cDNA amplicon accumulation of the therapeutic gene, namely the IL10 gene, in IMR-32 human neuroblastoma cell line (ATCC CCL-127) before its transfection and 48 hours after transfection of these cells with the DNA vector GDTT1.8NAS12-IL10 in order to assess the ability to penetrate into eukaryotic cells and functional activity, i.e. expression of the therapeutic gene at the mRNA level.

FIG. 3 shows curves of accumulation of amplicons during the reaction corresponding to:
1—cDNA of the IL10 gene in the IMR-32 cell line before transfection with the DNA vector GDTT1.8NAS12-IL10,
2—cDNA of the IL10 gene in the IMR-32 cell line after transfection with the DNA vector GDTT1.8NAS12-IL10,
3—cDNA of the B2M gene in the IMR-32 cell line before transfection with the DNA vector GDTT1.8NAS12-IL10,
4—cDNA of the B2M gene in the IMR-32 cell line after transfection with the DNA vector GDTT1.8NAS12-IL10.

The B2M (beta-2-microglobulin) gene listed in the GenBank database under number NM 004048.2 was used as a reference gene.

FIG. 4

shows diagrams of cDNA amplicon accumulation of the therapeutic gene, namely the IL13 gene, in U-118 MG human glioblastoma cell line (ATCC HTB-15) before its transfection and 48 hours after transfection of these cells with the DNA vector GDTT1.8NAS12-IL13 in order to assess the ability to penetrate into eukaryotic cells and functional activity, i.e. expression of the therapeutic gene at the mRNA level.

FIG. 4 shows curves of amplicon accumulation during the reaction corresponding to:
1—cDNA of the IL13 gene in the U-118 MG cells before transfection with the DNA vector GDTT1.8NAS12-IL13,
2—cDNA of the IL13 gene in the U-118 MG cells after transfection with the DNA vector GDTT1.8NAS12-IL13,
3—cDNA of the B2M gene in the U-118 MG cells before transfection with the DNA vector GDTT1.8NAS12-IL13,
4—cDNA of the B2M gene in the U-118 MG cells after transfection with the DNA vector GDTT1.8NAS12-IL13.

The B2M (beta-2-microglobulin) gene listed in the GenBank database under number NM 004048.2 was used as a reference gene.

FIG. 5

shows diagrams of cDNA amplicon accumulation of the therapeutic gene, namely the IFNB1 gene, in SH-SY5Y human neuroblastoma cell line (ATCC CRL-2266) before its transfection and 48 hours after transfection of these cells with the DNA vector GDTT1.8NAS12-IFNB1 in order to assess the ability to penetrate into eukaryotic cells and functional activity, i.e. expression of the therapeutic gene at the mRNA level.

FIG. 5 shows curves of amplicon accumulation during the reaction corresponding to:
1—cDNA of the IFNB1 gene in the SH-SY5Y cell line before transfection with the DNA vector GDTT1.8NAS12-IFNB1,
2—cDNA of the IFNB1 gene in the SH-SY5Y cell line after transfection with the DNA vector GDTT1.8NAS12-IFNB1,
3—cDNA of the B2M gene in the SH-SY5Y cell line before transfection with the DNA vector GDTT1.8NAS12-IFNB1,
4—cDNA of the B2M gene in the SH-SY5Y cell line after transfection with the DNA vector GDTT1.8NAS12-IFNB1.

The B2M (beta-2-microglobulin) gene listed in the GenBank database under number NM 004048.2 was used as a reference gene.

FIG. 6

shows diagrams of cDNA amplicon accumulation of the therapeutic gene, namely the TNFRSF4 gene, in SVG p12 human glial cell line (ATCC CRL-8621) before its transfection and 48 hours after transfection of these cells with the DNA vector GDTT1.8NAS12-TNFRSF4 in order to assess the ability to penetrate into eukaryotic cells and functional activity, i.e. expression of the therapeutic gene at the mRNA level.

FIG. 6 shows curves of amplicon accumulation during the reaction corresponding to:
1—cDNA of the TNFRSF4 gene in the SVG p12 cell line before transfection with the DNA vector GDTT1.8NAS12-TNFRSF4,
2—cDNA of the TNFRSF4 gene in the SVG p12 cell line after transfection with the DNA vector GDTT1.8NAS12-TNFRSF4,
3—cDNA of the B2M gene in the SVG p12 cell line before transfection with the DNA vector GDTT1.8NAS12-TNFRSF4,
4—cDNA of the B2M gene in the SVG p12 cell line after transfection with the DNA vector GDTT1.8NAS12-TNFRSF4.

The B2M (beta-2-microglobulin) gene listed in the GenBank database under number NM 004048.2 was used as a reference gene.

FIG. 7

shows diagrams of cDNA amplicon accumulation of the therapeutic gene, namely the TNFSF10 gene, in PFSK-1 human neuroectodermal tumour cell line (ATCC CRL-2060) before its transfection and 48 hours after transfection of these cells with the DNA vector GDTT1.8NAS12-TNFSF10 in order to assess the ability to penetrate into eukaryotic cells and functional activity, i.e. expression of the therapeutic gene at the mRNA level.

FIG. 7 shows curves of accumulation of amplicons during the reaction corresponding to:
1—cDNA of the TNFSF10 gene in the PFSK-1 line before transfection with the DNA vector GDTT1.8NAS12-TNFSF10,
2—cDNA of the TNFSF10 gene in the PFSK-1 line after transfection with the DNA vector GDTT1.8NAS12-TNFSF10,
3—cDNA of the B2M gene in the PFSK-1 line before transfection with the DNA vector GDTT1.8NAS12-TNFSF10,
4—cDNA of the B2M gene in the PFSK-1 line after transfection with the DNA vector GDTT1.8NAS12-TNFSF10.

The B2M (beta-2-microglobulin) gene listed in the GenBank database under number NM 004048.2 was used as a reference gene.

FIG. 8

shows diagrams of cDNA amplicon accumulation of the therapeutic gene, namely the BCL2 gene, in D341 Med human medulloblastoma cell line (ATCC HTB-187) before its transfection and 48 hours after transfection of these cells with the DNA vector GDTT1.8NAS12-BCL2 in order to assess the ability to penetrate into eukaryotic cells and functional activity, i.e. expression of the therapeutic gene at the mRNA level.

FIG. 8 shows curves of amplicon accumulation during the reaction corresponding to:
1—cDNA of the BCL2 gene in the D341 Med cells before transfection with the DNA vector GDTT1.8NAS12-BCL2,
2—cDNA of the BCL2 gene in the D341 Med cells after transfection with the DNA vector GDTT1.8NAS12-BCL2,
3—cDNA of the B2M gene in the D341 Med cells before transfection with the DNA vector GDTT1.8NAS12-BCL2,
4—cDNA of the B2M gene in the D341 Med cells after transfection with the DNA vector GDTT1.8NAS12-BCL2.

The B2M (beta-2-microglobulin) gene listed in the GenBank database under number NM 004048.2 was used as a reference gene.

FIG. 9

shows diagrams of cDNA amplicon accumulation of the therapeutic gene, namely the HGF gene, in A-172 human brain astrocytoma cell culture (ATCC CRL-1620) before its transfection and 48 hours after transfection of these cells with the DNA vector GDTT1.8NAS12-HGF in order to assess the ability to penetrate into eukaryotic cells and functional activity, i.e. expression of the therapeutic gene at the mRNA level.

FIG. 9 shows curves of amplicon accumulation during the reaction corresponding to:
1—cDNA of the HGF gene in the A-172 cell line before transfection with the DNA vector GDTT1.8NAS12-HGF,
2—cDNA of the HGF gene in the A-172 cell line after transfection with the DNA vector GDTT1.8NAS12-HGF,
3—cDNA of the B2M gene in the A-172 cell line before transfection with the DNA vector GDTT1.8NAS12-HGF,
4—cDNA of the B2M gene in the A-172 cell line after transfection with the DNA vector GDTT1.8NAS12-HGF.

The B2M (beta-2-microglobulin) gene listed in the GenBank database under number NM 004048.2 was used as a reference gene.

FIG. 10

shows diagrams of cDNA amplicon accumulation of the therapeutic gene, namely the IL-2 gene, in ATCC-BXS0117 human neural progenitor cell line (ATCC ACS-5003) before its transfection and 48 hours after transfection of these cells with the DNA vector GDTT1.8NAS12-IL2 in order to assess the ability to penetrate into eukaryotic cells and functional activity, i.e. expression of the therapeutic gene at the mRNA level.

FIG. 10 shows curves of amplicon accumulation during the reaction corresponding to:
1—cDNA of the IL-2 gene in the ATCC-BXS0117 cell line before transfection with the DNA vector GDTT1.8NAS12-IL2,
2—cDNA of the IL-2 gene in the ATCC-BXS0117 cell line after transfection with the DNA vector GDTT1.8NAS12-IL2,
3—cDNA of the B2M gene in the ATCC-BXS0117 cell line before transfection with the DNA vector GDTT1.8NAS12-IL2,
4—cDNA of the B2M gene in the ATCC-BXS0117 cell line after transfection with the DNA vector GDTT1.8NAS12-IL2.

The B2M (beta-2-microglobulin) gene listed in the GenBank database under number NM 004048.2 was used as a reference gene.

FIG. 11

shows the plot of the DDC protein concentration in the lysate of HCN-2 human cortical neuron cells (ATCC CRL-10742) after transfection of these cells with the DNA vector GDTT1.8NAS12-DDC in order to assess the functional activity, i.e. the therapeutic gene expression at the protein level, and the possibility of increasing the level of protein expression by gene therapy DNA vector based on the gene therapy DNA vector GDTT1.8NAS12 carrying the DDC therapeutic gene.

The following elements are indicated in FIG. 11:
culture A—the HCN-2 line transfected with aqueous dendrimer solution without plasmid DNA (reference),
culture B—the HCN-2 line transfected with the DNA vector GDTT1.8NAS12,
culture C—the HCN-2 line transfected with the DNA vector GDTT1.8NAS12-DDC.

FIG. 12

shows the plot of the IL10 protein concentration in the lysate of IMR-32 human neuroblastoma cell line (ATCC CCL-127) after transfection of these cells with the DNA vector GDTT1.8NAS12-IL10 in order to assess the functional activity, i.e. the therapeutic gene expression at the protein level, and the possibility of increasing the level of protein expression by gene therapy DNA vector based on the gene therapy DNA vector GDTT1.8NAS12 carrying the IL10 therapeutic gene.

The following elements are indicated in FIG. 12:
culture A—the IMR-32 line transfected with aqueous dendrimer solution without plasmid DNA (reference),
culture B—the IMR-32 line transfected with the DNA vector GDTT1.8NAS12,
culture C—the IMR-32 line transfected with the DNA vector GDTT1.8NAS12-IL10.

FIG. 13

shows the plot of the IL13 protein concentration in the lysate of U-118 MG human glioblastoma cell line (ATCC HTB-15) after transfection of these cells with the DNA vector GDTT1.8NAS12-IL13 in order to assess the functional activity, i.e. the therapeutic gene expression at the protein level, and the possibility of increasing the level of protein expression by gene therapy DNA vector based on the gene therapy DNA vector GDTT1.8NAS12 carrying the IL 13 therapeutic gene.

The following elements are indicated in FIG. 13:
culture A—the U-118 MG cell line transfected with aqueous dendrimer solution without plasmid DNA (reference),
culture B—the U-118 MG cell line transfected with the DNA vector GDTT1.8NAS12,
culture C—the U-118 MG cell line transfected with the DNA vector GDTT1.8NAS12-IL13.

FIG. 14

shows the plot of the IFNB1 protein concentration in the lysate of SH-SY5Y human neuroblastoma cell line (ATCC CRL-2266) after transfection of these cells with the DNA vector GDTT1.8NAS12-IFNB1 in order to assess the functional activity, i.e. the therapeutic gene expression at the protein level, and the possibility of increasing the level of protein expression by gene therapy DNA vector based on the gene therapy DNA vector GDTT1.8NAS12 carrying the IFNB 1 therapeutic gene.

The following elements are indicated in FIG. 14:
culture A—the SH-SY5Y cell line transfected with aqueous dendrimer solution without plasmid DNA (reference),
culture B—the SH-SY5Y cell line transfected with the DNA vector GDTT1.8NAS12,
culture C—the SH-SY5Y cell line transfected with the DNA vector GDTT1.8NAS12-IFNB1.

FIG. 15

shows the plot of the TNFRSF4 protein concentration in the lysate of SSVG p12 human glial cell line (ATCC CRL-8621) after transfection of these cells with the DNA vector GDTT1.8NAS12-TNFRSF4 in order to assess the functional activity, i.e. the therapeutic gene expression at the protein level, and the possibility of increasing the level of protein expression by gene therapy DNA vector based on the gene therapy DNA vector GDTT1.8NAS12 carrying the TNFRSF4 therapeutic gene.

The following elements are indicated in FIG. 15:
culture A—the SVG p12 cell line transfected with aqueous dendrimer solution without plasmid DNA (reference),
culture B—the SVG p12 cell line transfected with the DNA vector GDTT1.8NAS12,
culture C—the SVG p12 cell line transfected with the DNA vector GDTT1.8NAS12-TNFRSF4.

FIG. 16

shows the plot of the TNFSF10 protein concentration in the lysate of PFSK-1 human neuroectodermal tumour cell line (ATCC CRL-2060) after transfection of these cells with the DNA vector GDTT1.8NAS12-TNFSF10 in order to assess the functional activity, i.e. the therapeutic gene expression at the protein level, and the possibility of increasing the level of protein expression by gene therapy DNA vector based on the gene therapy DNA vector GDTT1.8NAS12 carrying the TNFSF10 therapeutic gene.

The following elements are indicated in FIG. 16:
culture A—the PFSK-1 line transfected with aqueous dendrimer solution without plasmid DNA (reference),
culture B—the PFSK-1 line transfected with the DNA vector GDTT1.8NAS12,
culture C—the PFSK-1 line transfected with the DNA vector GDTT1.8NAS12-TNFSF10.

FIG. 17

shows the plot of BCL2 protein concentration in the lysate of D341 Med human medulloblastoma cell line (ATCC HTB-187) after transfection of these cells with the DNA vector GDTT1.8NAS12-BCL2 in order to assess the functional activity, i.e. the therapeutic gene expression at the protein level, and the possibility of increasing the level of protein expression by gene therapy DNA vector based on the gene therapy DNA vector GDTT1.8NAS12 carrying the BCL2 therapeutic gene.

The following elements are indicated in FIG. 17:
culture A—the D341 Med cell line transfected with aqueous dendrimer solution without plasmid DNA (reference),
culture B—the D341 Med cell line transfected with the DNA vector GDTT1.8NAS12,
culture C—the D341 Med cell line transfected with the DNA vector GDTT1.8NAS12-BCL2.

FIG. 18

shows the plot of the HGF protein concentration in the lysate of A-172 human brain astrocytoma cell culture (ATCC CRL-1620) after transfection of these cells with the DNA vector GDTT1.8NAS12-HGF in order to assess the functional activity, i.e. the therapeutic gene expression at the protein level, and the possibility of increasing the level of protein expression by gene therapy DNA vector based on the gene therapy DNA vector GDTT1.8NAS12 carrying the HGF therapeutic gene.

The following elements are indicated in FIG. 18:
culture A—the A-172 cell line transfected with aqueous dendrimer solution without plasmid DNA (reference),
culture B—the A-172 cell line transfected with the DNA vector GDTT1.8NAS12,
culture C—the A-172 cell line transfected with the DNA vector GDTT1.8NAS12-HGF.

FIG. 19

shows the plot of the IL-2 protein concentration in the lysate of ATCC-BXS0117 human neural progenitor cell line (ATCC ACS-5003) after transfection of these cells with the DNA vector GDTT1.8NAS12-IL2 in order to assess the functional activity, i.e. the therapeutic gene expression at the protein level, and the possibility of increasing the level of protein expression by gene therapy DNA vector based on the gene therapy DNA vector GDTT1.8NAS12 carrying the IL-2 therapeutic gene.

The following elements are indicated in FIG. 19:
culture A—the ATCC-BXS0117 cell line transfected with aqueous dendrimer solution without plasmid DNA (reference),
culture B—the ATCC-BXS0117 cell line transfected with the DNA vector GDTT1.8NAS12,
culture C—the ATCC-BXS0117 cell line transfected with the DNA vector GDTT1.8NAS12-IL2.

FIG. 20

shows the plot of the IFNB1 protein concentration in the skin biopsy samples of three patients after intradermal injection of the gene therapy DNA vector GDTT1.8NAS12-IFNB1 in order to assess the functional activity, i.e. the expression of the therapeutic gene at the protein level, and the possibility of increasing the level of protein expression using the gene therapy DNA vector based on the gene therapy DNA vector GDTT1.8NAS12 carrying the IFNB1 therapeutic gene.

The following elements are indicated in FIG. 20:
P1I is patient P1 skin biopsy in the region of injection of the gene therapy DNA vector GDTT1.8NAS12-IFNB1,
P1II is patient P1 skin biopsy in the region of injection of the gene therapy DNA vector GDTT1.8NAS12 (placebo),
P1III is patient P1 skin biopsy from an intact site,
P2I is patient P2 skin biopsy in the region of injection of the gene therapy DNA vector GDTT1.8NAS12-IFNB1,
P2II is patient P2 skin biopsy in the region of injection of gene therapy DNA vector GDTT1.8NAS12 (placebo),
P2III is patient P2 skin biopsy from an intact site,
P3I is patient P3 skin biopsy in the region of injection of the gene therapy DNA vector GDTT1.8NAS12-IFNB1,
P3II is patient P3 skin biopsy in the region of injection of the gene therapy DNA vector GDTT1.8NAS12 (placebo),
P3III is patient P3 skin biopsy from an intact site.

FIG. 21

shows the plot of the IL13 protein concentration in the gastrocnemius muscle biopsy specimens of three patients after injection of the gene therapy DNA vector GDTT1.8NAS12-IL13 into the gastrocnemius muscle of these patients in order to assess the functional activity, i.e. the therapeutic gene expression at the protein level, and the possibility of increasing the level of protein expression using the gene therapy DNA vector based on the gene therapy DNA vector GDTT1.8NAS12 carrying the IL13 therapeutic gene.

The following elements are indicated in FIG. 21:
P1I is patient P1 gastrocnemius muscle biopsy in the region of injection of the gene therapy DNA vector GDTT1.8NAS12-IL13,
P1II is patient P1 gastrocnemius muscle biopsy in the region of injection of the gene therapy DNA vector GDTT1.8NAS12 (placebo),
P1III is patient P1 gastrocnemius muscle biopsy from an intact site,
P2I is patient P2 gastrocnemius muscle biopsy in the region of injection of the gene therapy DNA vector GDTT1.8NAS12-IL13,
P2II is patient P2 gastrocnemius muscle biopsy in the region of injection of the gene therapy DNA vector GDTT1.8NAS12 (placebo),
P2III is patient P2 gastrocnemius muscle biopsy from an intact site,
P3I is patient P3 gastrocnemius muscle biopsy in the region of injection of the gene therapy DNA vector GDTT1.8NAS12-IL13,
P3II is patient P3 gastrocnemius muscle biopsy in the region of injection of the gene therapy DNA vector GDTT1.8NAS12 (placebo),
P3III is patient P3 gastrocnemius muscle biopsy from an intact site.

FIG. 22

shows the plot of the IL-2 protein concentration in the skin biopsy samples of three patients after intradermal injection of the gene therapy DNA vector GDTT1.8NAS12-IL2 in order to assess the functional activity, i.e. the expression of the therapeutic gene at the protein level, and the possibility of increasing the level of protein expression using the gene therapy DNA vector based on the gene therapy DNA vector GDTT1.8NAS12 carrying the IL-2 therapeutic gene.

The following elements are indicated in FIG. 22:
P1I is patient P1 skin biopsy in the region of injection of the gene therapy DNA vector GDTT1.8NAS12-IL2,
P1II is patient P1 skin biopsy in the region of injection of the gene therapy DNA vector GDTT1.8NAS12 (placebo),
P1III is patient P1 skin biopsy from an intact site,
P2I is patient P2 skin biopsy in the region of injection of the gene therapy DNA vector GDTT1.8NAS12-IL2,
P2II is patient P2 skin biopsy in the region of injection of gene therapy DNA vector GDTT1.8NAS12 (placebo),
P2III is patient P2 skin biopsy from an intact site,
P3I is patient P3 skin biopsy in the region of injection of the gene therapy DNA vector GDTT1.8NAS12-IL2,
P3II is patient P3 skin biopsy in the region of injection of the gene therapy DNA vector GDTT1.8NAS12 (placebo),
P3III is patient P3 skin biopsy from an intact site.

FIG. 23

shows the plot of the IL10 protein concentration in human skin biopsy samples after intradermal injection of autologous fibroblast cell line transfected with the gene therapy DNA vector GDTT1.8NAS12-IL10 in order to demonstrate the method of use by injecting autologous cells transfected with the gene therapy DNA vector GDTT1.8NAS12-IL10.

The following elements are indicated in FIG. 23:
P1C is patient P1 skin biopsy in the region of injection of autologous fibroblast cell line of the patient transfected with the gene therapy DNA vector GDTT1.8NAS12-IL10,
P1B is patient P1 skin biopsy in the region of injection of autologous fibroblasts of the patient transfected with the gene therapy DNA vector GDTT1.8NAS12,
P1A is patient P1 skin biopsy from an intact site.

FIG. 24

shows the plot of concentrations of human DDC protein, human BCL2 protein, human HGF protein, human TNFSF10 protein, and human TNFRSF4 protein in muscle tissue of three groups of Wistar rats after injection of a mixture of the following gene therapy vectors: the gene therapy DNA vector GDTT1.8NAS12-DDC, the gene therapy DNA vector GDTT1.8NAS12-BCL2, the gene therapy DNA vector GDTT1.8NAS12-HGF, the gene therapy DNA vector GDTT1.8NAS12-TNFSF10, and the gene therapy DNA vector GDTT1.8NAS12-TNFRSF4 in order to demonstrate the method of use of a mixture of gene therapy DNA vectors.

The following elements are indicated in FIG. 24:
K1I is a fragment of muscle tissue of rat I from the K1 group in the region of injection of the mixture of the following gene therapy DNA vectors: GDTT1.8NAS12-DDC, GDTT1.8NAS12-BCL2, GDTT1.8NAS12-HGF, GDTT1.8NAS12-TNFSF10, and GDTT1.8NAS12-TNFRSF4,
K1II is a fragment of muscle tissue of rat II from the K1 group in the region of injection of the mixture of the following gene therapy DNA vectors: GDTT1.8NAS12-DDC, GDTT1.8NAS12-BCL2, GDTT1.8NAS12-HGF, GDTT1.8NAS12-TNFSF10, and GDTT1.8NAS12-TNFRSF4,
K1III is a fragment of muscle tissue of rat III from the K1 group in the region of injection of the mixture of the following gene therapy DNA vectors: GDTT1.8NAS12-DDC, GDTT1.8NAS12-BCL2, GDTT1.8NAS12-HGF, GDTT1.8NAS12-TNFSF10, and GDTT1.8NAS12-TNFRSF4,
K1IV is a fragment of muscle tissue of rat IV from the K1 group in the region of injection of the mixture of the following gene therapy DNA vectors: GDTT1.8NAS12-DDC, GDTT1.8NAS12-BCL2, GDTT1.8NAS12-HGF, GDTT1.8NAS12-TNFSF10, and GDTT1.8NAS12-TNFRSF4,
K1V is a fragment of muscle tissue of rat V from the K1 group in the region of injection of the mixture of the following gene therapy DNA vectors: GDTT1.8NAS12-DDC, GDTT1.8NAS12-BCL2, GDTT1.8NAS12-HGF, GDTT1.8NAS12-TNFSF10, and GDTT1.8NAS12-TNFRSF4,
K2I is a fragment of muscle tissue of rat I from the K2 group in the region of injection of the gene therapy DNA vector GDTT1.8NAS12 (placebo),
K2II is a fragment of muscle tissue of rat II from the K2 group in the region of injection of the gene therapy DNA vector GDTT1.8NAS12 (placebo),
K2III is a fragment of muscle tissue of rat III from the K2 group in the region of injection of the gene therapy DNA vector GDTT1.8NAS12 (placebo),
K2IV is a fragment of muscle tissue of rat IV from the K2 group in the region of injection of the gene therapy DNA vector GDTT1.8NAS12 (placebo),
K2V is a fragment of muscle tissue of rat V from the K2 group in the region of injection of the gene therapy DNA vector GDTT1.8NAS12 (placebo),
K31 is a fragment of muscle tissue of rat I from the K3 group from a reference intact site,
K3II is a fragment of muscle tissue of rat II from the K3 group from a reference intact site,
K3III is a fragment of muscle tissue of rat III from the K3 group from a reference intact site,
K3IV is a fragment of muscle tissue of rat IV from the K3 group from a reference intact site,
K3V is a fragment of muscle tissue of rat V from the K3 group from a reference intact site.

FIG. 25

shows diagrams of cDNA amplicon accumulation of the IL13 therapeutic gene in bovine peripheral blood mononuclear cells (PBMC) before and 48 hours after transfection of these cells with the DNA vector GDTT1.8NAS12-IL13 in order to demonstrate the method of use by injecting the gene therapy DNA vector in animals.

FIG. 25 shows curves of amplicon accumulation during the reaction corresponding to:
1—cDNA of the IL13 gene in bovine PBMC cells before transfection with the gene therapy DNA vector GDTT1.8NAS12-IL13,
2—cDNA of the IL13 gene in bovine PBMC cells after transfection with the gene therapy DNA vector GDTT1.8NAS12-IL13,
3—cDNA of the ACT gene in bovine PBMC cells before transfection with the gene therapy DNA vector GDTT1.8NAS12-IL13,
4—cDNA of the ACT gene in bovine PBMC cells after transfection with the gene therapy DNA vector GDTT1.8NAS12-IL13.

Bull/cow actin gene (ACT) listed in the GenBank database under number AH001130.2 was used as a reference gene.

### DESCRIPTION OF THE INVENTION

Gene therapy DNA vectors carrying human therapeutic genes designed to increase the expression level of these therapeutic genes in human and animal tissues were constructed based on 2591 bp DNA vector GDTT1.8NAS12. The method of production of each gene therapy DNA vector carrying the therapeutic genes is to clone the protein coding sequence of the therapeutic gene selected from the group of the following genes: human DDC gene (encodes DDC protein), human IL10 gene (encodes IL10 protein), human IL13 gene (encodes IL13 protein), human IFNB1 gene (encodes IFNB1 protein), human TNFRSF4 gene (encodes TNFRSF4 protein), human TNFSF10 gene (encodes TNFSF10 protein), human BCL2 gene (encodes BCL2 protein), human HGF gene (encodes HGF protein), human IL-2 gene (encodes IL-2 protein), to the polylinker of the gene therapy DNA vector GDTT1.8NAS12. The ability of DNA vectors to penetrate into eukaryotic cells is known to be due mainly to the vector size. DNA vectors with the smallest size have higher penetration capability. Thus, the absence of elements in the vector that bear no functional load but at the same time increase the vector DNA size is preferred. These features of DNA vectors were taken into account during the production of the gene therapy DNA vectors based on the gene therapy DNA vector GDTT1.8NAS12 carrying the therapeutic gene selected from the group of DDC, IL10, IL13, IFNB1, TNFRSF4, TNFSF10, BCL2, HGF, and IL-2 genes with no large non-functional sequences and antibiotic resistance genes in the vector, which, in addition to technological advantages and safe use, allowed for the significant reduction of size of the produced gene therapy DNA vector GDTT1.8NAS12 carrying the therapeutic gene selected from the group of DDC, IL10, IL13, IFNB1, TNFRSF4, TNFSF10, BCL2, HGF, and IL-2 genes. Thus, the ability of the obtained gene therapy DNA vector to penetrate into eukaryotic cells is due to its small length.

Each of the following gene therapy DNA vectors: GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, was produced as follows: the coding region of the therapeutic gene from the group of DDC, or IL10, or IL13, or IFNB1, or TNFRSF4, or TNFSF10, or BCL2, or HGF, or IL-2 gene was cloned to the gene therapy DNA vector GDTT1.8NAS12, and the gene therapy DNA vector GDTT1.8NAS12-DDC, SEQ ID No. 1, or GDTT1.8NAS12-IL10, SEQ ID No. 2 or GDTT1.8NAS12-IL13, SEQ ID No. 3, or GDTT1.8NAS12-IFNB1, SEQ ID No. 4, or GDTT1.8NAS12-TNFRSF4, SEQ ID No. 5, or GDTT1.8NAS12-TNFSF10, SEQ ID No. 6, or GDTT1.8NAS12-BCL2, SEQ ID No. 7, or GDTT1.8NAS12-HGF, SEQ ID No. 8, or GDTT1.8NAS12-IL2, SEQ ID No. 9, respectively, was obtained. The coding region of the DDC gene (1447 bp), or the IL10 gene (540 bp), or the IL13 gene (444 bp), or the IFNB1 gene (567 bp), or the TNFRSF4 gene (837 bp), or the TNFSF10 gene (849 bp), or the BCL2 gene (724 bp), or the HGF gene (2190 bp), or the IL-2 gene (465 bp) was produced by extracting total RNA from the biological tissue sample of a healthy human. The reverse transcription reaction was used for the synthesis of the first cDNA chain of human DDC, IL10, IL13, IFNB1, TNFRSF4, TNFSF10, BCL2, HGF, and IL-2 genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector GDTT1.8NAS12 was performed by BamHI, EcoRI, HindIII, KpnI, and SalI restriction sites located in the GDTT1.8NAS12 vector polylinker. The selection of restriction sites was carried out in such a way that the cloned fragment entered the reading frame of expression cassette of the vector GDTT1.8NAS12, while the protein coding sequence did not contain restriction sites for the selected endonucleases. Experts in this field realise that the methodological implementation of the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 production can vary in terms of selection of known methods of molecular gene cloning, and these methods are included in the scope of this invention. For example, different oligonucleotide sequences can be used to amplify the DDC, or IL10, or IL13, or IFNB1, or TNFRSF4, or TNFSF10, or BCL2, or HGF, or IL-2 gene, as well as different restriction endonucleases or laboratory techniques, such as ligation independent cloning of genes.

The gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 has the nucleotide sequence SEQ ID No. 1, or SEQ ID No. 2, or SEQ ID No. 3, or SEQ ID No. 4, or SEQ ID No. 5, SEQ ID No. 6, or SEQ ID No. 7, or SEQ ID No. 8, or SEQ ID No. 9, respectively. Also, experts in this field are aware of the degeneracy property of genetic code, and therefore the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of the GDTT1.8NAS12 vector. Also, experts in this field are aware of the genetic polymorphism, and therefore the scope of this invention also includes variants of nucleotide sequences of genes from the group of DDC, IL10, IL13, IFNB1, TNFRSF4, TNFSF10, BCL2, HGF, and IL-2 genes that also encode different variants of the amino acid sequences of DDC, IL10, IL13, IFNB1, TNFRSF4, TNFSF10, BCL2, HGF, or IL-2 proteins that do not differ from those listed in their functional activity under physiological conditions.

The ability to penetrate into eukaryotic cells and express functional activity, i.e. the ability to express the therapeutic gene of the obtained gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 is confirmed by injecting the obtained vector into eukaryotic cells and subsequent analysis of the expression of specific mRNA and/or protein product of the therapeutic gene. The presence of specific mRNA in cells into which the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 was injected shows the ability of the obtained vector to both penetrate into eukaryotic cells and express mRNA of the therapeutic gene. Furthermore, it is known to the experts in this field that the presence of mRNA gene is a mandatory condition but not an evidence of the translation of protein encoded by the therapeutic gene. Therefore, in order to confirm properties of the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 to express the therapeutic gene at the protein level in eukaryotic cells into which the gene therapy DNA vector was injected, analysis of the concentration of proteins encoded by the therapeutic genes was carried out using immunological methods. The presence of the DDC, or IL10, or IL13, or IFNB1, or TNFRSF4, or TNFSF10, or BCL2, or HGF, or IL-2 protein confirms the efficiency of expression of therapeutic genes in eukaryotic cells and the possibility of increasing the protein concentration using the gene therapy DNA vector based on the gene therapy DNA vector GDTT1.8NAS12 carrying the therapeutic gene selected from the group of DDC, IL10, IL13, IFNB1, TNFRSF4, TNFSF10, BCL2, HGF, and IL-2 genes. Thus, in order to confirm the expression efficiency of the gene therapy DNA GDTT1.8NAS12-DDC carrying the therapeutic gene, namely the DDC gene, the gene therapy DNA vector GDTT1.8NAS12-IL10 carrying the therapeutic gene, namely the IL10 gene, the gene therapy DNA vector GDTT1.8NAS12-IL13 carrying the therapeutic gene, namely the IL13 gene, the gene therapy DNA vector GDTT1.8NAS12-IFNB1 carrying the therapeutic gene, namely the IFNB1 gene, the gene therapy DNA vector GDTT1.8NAS12-TNFRSF4 carrying the therapeutic gene, namely the TNFRSF4 gene, the gene therapy DNA vector GDTT1.8NAS12-TNFSF10 carrying the therapeutic gene, namely the TNFSF10 gene, the gene therapy DNA vector GDTT1.8NAS12-BCL2 carrying the therapeutic gene, namely the BCL2 gene, the gene therapy DNA vector GDTT1.8NAS12-HGF carrying the therapeutic gene, namely the HGF gene, the gene therapy DNA vector GDTT1.8NAS12-IL2 carrying the therapeutic gene, namely the IL-2 gene, the following methods were used:
A) Real-time PCR, i.e. change in accumulation of therapeutic gene mRNA amplicons in human and animal cell lysate after transfection of different human and animal cell lines with the gene therapy DNA vectors,
B) Enzyme-linked immunosorbent assay, i.e. change in the quantitative level of therapeutic proteins in the human cell lysate after transfection of different human cell lines with the gene therapy DNA vectors,
C) Enzyme-linked immunosorbent assay, i.e. change in the quantitative level of therapeutic proteins in the supernatant of human and animals tissue biopsy specimens after the injection of the gene therapy DNA vectors into these tissues,
D) Enzyme-linked immunosorbent assay, i.e. change in the quantitative level of therapeutic proteins in the supernatant of human tissue biopsies after the injection of these tissues with autologous cells of this human transfected with the gene therapy DNA vectors.

In order to confirm the practicability of use of the constructed gene therapy DNA vector GDTT1.8NAS12-DDC carrying the therapeutic gene, namely the DDC gene, the gene therapy DNA vector GDTT1.8NAS12-IL10 carrying the therapeutic gene, namely the IL10 gene, the gene therapy DNA vector GDTT1.8NAS12-IL13 carrying the therapeutic gene, namely the IL13 gene, the gene therapy DNA vector GDTT1.8NAS12-IFNB1 carrying the therapeutic gene, namely the IFNB1 gene, the gene therapy DNA vector GDTT1.8NAS12-TNFRSF4 carrying the therapeutic gene, namely the TNFRSF4 gene, the gene therapy DNA vector GDTT1.8NAS12-TNFSF10 carrying the therapeutic gene, namely the TNFSF10 gene, the gene therapy DNA vector GDTT1.8NAS12-BCL2 carrying the therapeutic gene, namely the BCL2 gene, the gene therapy DNA vector GDTT1.8NAS12-HGF carrying the therapeutic gene, namely the HGF gene, the gene therapy DNA vector GDTT1.8NAS12-IL2 carrying the therapeutic gene, namely the IL-2 gene, the following was performed:
A) transfection of different human and animal cell lines with the gene therapy DNA vectors,
B) injection of the gene therapy DNA vectors into different human and animal tissues,
C) injection of a mixture of the gene therapy DNA vectors into animal tissues,
D) injection of autologous cells transfected with the gene therapy DNA vectors into human tissues.

These methods of use are free of potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of the gene therapy DNA vector GDTT1.8NAS12-DDC, or the gene therapy DNA vector GDTT1.8NAS12-IL10, or the gene therapy DNA vector GDTT1.8NAS12-IL13, or the gene therapy DNA vector GDTT1.8NAS12-IFNB1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2 (SEQ ID No. 1, or SEQ ID No. 2, or SEQ ID No. 3, or SEQ ID No. 4, or SEQ ID No. 5, or SEQ ID No. 6, or SEQ ID No. 7, or SEQ ID No. 8, or SEQ ID No. 9, respectively).

It is known to the experts in this field that antibiotic resistance genes in the gene therapy DNA vectors are used to obtain these vectors in preparative quantities by increasing bacterial biomass in a nutrient medium containing a selective antibiotic. Within this invention, in order to ensure the safe use of the gene therapy DNA vector GDTT1.8NAS12 carrying the DDC, or IL10, or IL13, or IFNB1, or TNFRSF4, or TNFSF10, or BCL2, or HGF, or IL-2 therapeutic genes, the use of selective nutrient media containing an antibiotic is not possible. A method for obtaining strains for production of the gene therapy vectors based on Escherichia coli strain JM110-NAS is proposed as a technological solution for obtaining the gene therapy DNA vector GDTT1.8NAS12 carrying a therapeutic gene selected from the group of DDC, IL10, IL13, IFNB1, TNFRSF4, TNFSF10, BCL2, HGF, and IL-2 genes in order to scale up the production of the gene therapy vectors on an industrial scale. The method of Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.8NAS12-IFNB1, or Escherichia coli strain JM110-NAS/GDTT1.8NAS12-TNFRSF4, or Escherichia coli strain JM110-NAS/GDTT1.8NAS12-TNFSF10, or Escherichia coli strain JM110-NAS/GDTT1.8NAS12-BCL2, or Escherichia coli strain JM110-NAS/GDTT1.8NAS12-HGF, or Escherichia coli strain JM110-NAS/GDTT1.8NAS12-IL2 production involves production of competent cells of Escherichia coli strain JM110-NAS with the injection of the gene therapy DNA vector GDTT1.8NAS12-DDC, or the gene therapy DNA vector GDTT1.8NAS12-IL10, or the gene therapy DNA vector GDTT1.8NAS12-IL13, or the gene therapy DNA vector GDTT1.8NAS12-IFNB1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2 into these cells, respectively, using transformation (electroporation) methods widely known to experts in this field. The strains obtained, i.e. the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2 is used for production of the gene therapy DNA vector GDTT1.8NAS12-DDC, or the gene therapy DNA vector GDTT1.8NAS12-IL10, or the gene therapy DNA vector GDTT1.8NAS12-IL13, or the gene therapy DNA vector GDTT1.8NAS12-IFNB 1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2, respectively, allowing for the use of antibiotic-free media.

In order to confirm the production of the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2, transformation, selection, and subsequent biomass growth with extraction of plasmid DNA were performed.

To confirm the producibility and constructability and scale up of the production of the gene therapy DNA vector GDTT1.8NAS12-DDC carrying the therapeutic gene, namely the DDC gene, the gene therapy DNA vector GDTT1.8NAS12-IL10 carrying the therapeutic gene, namely the IL10 gene, the gene therapy DNA vector GDTT1.8NAS12-IL13 carrying the therapeutic gene, namely the IL13 gene, the gene therapy DNA vector GDTT1.8NAS12-IFNB1 carrying the therapeutic gene, namely the IFNB1 gene, the gene therapy DNA vector GDTT1.8NAS12-TNFRSF4 carrying the therapeutic gene, namely the TNFRSF4 gene, the gene therapy DNA vector GDTT1.8NAS12-TNFSF10 carrying the therapeutic gene, namely the TNFSF10 gene, the gene therapy DNA vector GDTT1.8NAS12-BCL2 carrying the therapeutic gene, namely the BCL2 gene, the gene therapy DNA vector GDTT1.8NAS12-HGF carrying the therapeutic gene, namely the HGF gene, the gene therapy DNA vector GDTT1.8NAS12-IL2 carrying the therapeutic gene, namely the IL-2 gene, to an industrial scale, the fermentation on an industrial scale of the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2, each containing the gene therapy DNA vector GDTT1.8NAS12 carrying the therapeutic gene, namely the DDC, or the IL10, or the IL13, or the IFNB1, or the TNFRSF4, or the TNFSF10, or the BCL2, or the HGF, or the IL-2 gene, was performed.

The method of scaling the production of bacterial mass to an industrial scale for the isolation of the gene therapy DNA vector GDTT1.8NAS12 carrying the therapeutic gene selected from the group of DDC, IL10, IL13, IFNB1, TNFRSF4, TNFSF10, BCL2, HGF, and IL-2 genes involves incubation of the seed culture of the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2 in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. the gene therapy DNA vector GDTT1.8NAS12-DDC, or the gene therapy DNA vector GDTT1.8NAS12-IL10, or the gene therapy DNA vector GDTT1.8NAS12-IL13, or the gene therapy DNA vector GDTT1.8NAS12-IFNB1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2 is extracted, multi-stage filtered, and purified by chromatographic methods. It is known to the experts in this field that culture conditions of strains, composition of nutrient media (except for antibiotic-free), equipment used, and DNA purification methods may vary within the standard operating procedures depending on the particular production line, but known approaches to scaling, industrial production, and purification of DNA vectors using the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2 fall within the scope of this invention.

The described disclosure of the invention is illustrated by examples of the embodiment of this invention.

The essence of the invention is explained in the following examples.

### Example 1

Production of the gene therapy DNA vector GDTT1.8NAS12-DDC carrying the therapeutic gene, namely the DDC gene.

The gene therapy DNA vector GDTT1.8NAS12-DDC was constructed by cloning the coding region of the DDC gene (1447 bp) to a 2591 bp DNA vector GDTT1.8NAS12 by SalI and HindIII restriction sites. The coding region of the DDC gene (1447 bp) was obtained by isolating total RNA from the biological human tissue sample followed by reverse transcription reaction using commercial kit Mint-2 (Evrogen, Russia) and PCR amplification using the following oligonucleotides:

DDC-F
ATCGTCGACCACCATGAACGCAAGTGAGTTCCGA,
DDC-R
ACCAAGCTTCTACTCCCTCTCTG,

and commercially available kit Phusion® High-Fidelity DNA Polymerase (New England Biolabs, USA).

The gene therapy DNA vector GDTT1.8NAS12 was constructed by consolidating six fragments of DNA derived from different sources:
(a) the origin of replication was produced by PCR amplification of a region of commercially available pUC19 using UCori-Bam and UCori-Nco oligonucleotides (List of Oligonucleotides Sequences, (1)-(2)),
(b) hGH-TA transcription terminator was obtained by PCR amplification of a site of human genomic DNA using hGH-F and hGH-R oligonucleotides (List of Oligonucleotides Sequences, (3) and (4)),
(c) the RNA-out regulatory site of transposon Tn10 was synthesised from RO-F, RO-R, RO-1, RO-2, and RO-3 oligonucleotides (List of Oligonucleotides Sequences, (5)-(9)),
(d) the kanamycin resistance gene was obtained by PCR amplification of a site of commercially available human pET-28 plasmid using Kan-F and Kan-R oligonucleotides (List of Oligonucleotides Sequences, (10) and (11)),
(e) the polylinker was obtained by radiolabelling and annealing of four synthetic oligonucleotides, MCS1, MCS2, MCS3, and MCS4 (List of Oligonucleotides Sequences, (12)-(15)),
(f) promoter/regulator region of EF1a human elongation factor gene with its own enhancer was obtained by PCR amplification of a site of human genomic DNA using EF1-Xho and EF1-R oligonucleotides (List of Oligonucleotides Sequences, (16)-(17)).

PCR amplification was performed using the commercially available kit Phusion® High-Fidelity DNA Polymerase (New England Biolabs) as per the manufacturer's instructions. The fragments (b), (c), and (d) had overlapping regions allowing for their consolidation with subsequent PCR amplification. Fragments (b), (c), and (d) were consolidated using hGH-F and Kan-R oligonucleotides (List of Oligonucleotides Sequences, (3) and (11)). Afterwards, the obtained DNA fragments were consolidated by restriction with subsequent ligation by BamHI and NcoI sites. This resulted in a vector still devoid of the polylinker. To add it, the plasmid was cleaved by restriction endonucleases by BamHI and EcoRI sites with further ligation to fragment (e). This resulted in a 2408 bp intermediate vector carrying a kanamycin resistance gene, but still without promoter/regulator site of elongation factor EF1a gene with its own enhancer. The obtained vector was cleaved by restriction endonucleases by SalI and BamHI sites with further ligation to fragment (f). This resulted in a 3608 bp vector carrying a kanamycin resistance gene and promoter/regulator site of elongation factor EF1a gene with its own enhancer. Then the kanamycin resistance gene was cleaved by SpeI restriction sites, and the remaining fragment was ligated to itself. This resulted in a 2591 bp recombinant gene therapy DNA vector GDTT1.8NAS12 enabling antibiotic-free selection and expression of therapeutic genes cloned into it in the most types of human and animal tissues.

The amplification product of the coding region of the DDC gene and the DNA vector GDTT1.8NAS12 was cleaved by restriction endonucleases SalI and HindIII.

This resulted in a 4006 bp DNA vector GDTT1.8NAS12-DDC with the nucleotide sequence SEQ ID No. 1 and general structure shown in FIG. 1A.

### Example 2

Production of the gene therapy DNA vector GDTT1.8NAS12-IL10 carrying the therapeutic gene, namely the IL10 gene.

The gene therapy DNA vector GDTT1.8NAS12-IL10 was constructed by cloning the coding region of the IL10 gene (540 bp) to a 2591 bp DNA vector GDTT1.8NAS12 by BamHI and SalI restriction sites. The coding region of the IL10 gene (540 bp) was obtained by isolating total RNA from the biological human tissue sample followed by reverse transcription reaction using commercial kit Mint-2 (Evrogen, Russia) and PCR amplification using the following oligonucleotides:

IL10-F
AGGATCCACCATGCACAGCTCAGCACTGC,
IL10-R
CTTGTCGACTCAGTTTCGTATCTTCATTGTC,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 3093 bp DNA vector GDTT1.8NAS12-IL10 with the nucleotide sequence SEQ ID No. 2 and general structure shown in FIG. 1B.

The gene therapy DNA vector GDTT1.8NAS12 was constructed as described in Example 1.

### Example 3

Production of the gene therapy DNA vector GDTT1.8NAS12-IL13 carrying the therapeutic gene, namely the human IL13 gene.

The gene therapy DNA vector GDTT1.8NAS12-IL13 was constructed by cloning the coding region of the IL13 gene (444 bp) to a 2591 bp DNA vector GDTT1.8NAS12 by BamHI and SalI restriction sites. The coding region of the IL13 gene (444 bp) was obtained by isolating total RNA from the biological human tissue sample followed by reverse transcription reaction using commercial kit Mint-2 (Evrogen, Russia) and PCR amplification using the following oligonucleotides:

IL13-F
AGGATCCACCATGCATCCGCTCCTCAATC,
IL13-R
CTTGTCGACTCAGTTGAACTGTCCCTCG,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 2997 bp DNA vector GDTT1.8NAS12-IL13 with the nucleotide sequence SEQ ID No. 3 and general structure shown in FIG. 1C.

The gene therapy DNA vector GDTT1.8NAS12 was constructed as described in Example 1.

### Example 4

Production of the gene therapy DNA vector GDTT1.8NAS12-IFNB1 carrying the therapeutic gene, namely the IFNB1 gene.

The gene therapy DNA vector GDTT1.8NAS12-IFNB1 was constructed by cloning the coding region of the IFNB1 gene (567 bp) to a 2591 bp DNA vector GDTT1.8NAS12 by BamHI and SalI restriction sites. The coding region of the IFNB1 gene (567 bp) was obtained by isolating total RNA from the biological human tissue sample followed by reverse transcription reaction using commercial kit Mint-2 (Evrogen) and PCR amplification using the following oligonucleotides:

IFNB1-F
AAAGGATCCACCATGACCAACAAGTGTCTCCTCCAAA,
IFNB1-R
TTTGTCGACTCAGTTTCGGAGGTAACCTGTAAGTCTG,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 3120 bp DNA vector GDTT1.8NAS12-IFNB1 with the nucleotide sequence SEQ ID No. 4 and general structure shown in FIG. 1D.

The gene therapy DNA vector GDTT1.8NAS12 was constructed as described in Example 1.

### Example 5

Production of the gene therapy DNA vector GDTT1.8NAS12-TNFRSF4 carrying the therapeutic gene, namely the TNFRSF4 gene.

The gene therapy DNA vector GDTT1.8NAS12-TNFRSF4 was constructed by cloning the coding region of the TNFRSF4 gene (837 bp) to a 2591 bp DNA vector GDTT1.8NAS12 by BamHI and SalI restriction sites. The coding region of the TNFRSF4 gene (837 bp) was obtained by isolating total RNA from the biological human tissue sample followed by reverse transcription reaction using commercial kit Mint-2 (Evrogen) and PCR amplification using the following oligonucleotides:

TNFRSF4-F
AGGATCCACCATGTGCGTGGGGGCTCGG,
TNFRSF4-R
CTTGTCGACTCAGATCTTGGCCAGGGTG,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 3390 bp DNA vector GDTT1.8NAS12-TNFRSF4 with the nucleotide sequence SEQ ID No. 5 and general structure shown in FIG. 1E.

The gene therapy DNA vector GDTT1.8NAS12 was constructed as described in Example 1.

### Example 6

Production of the gene therapy DNA vector GDTT1.8NAS12-TNFSF10 carrying the therapeutic gene, namely the TNFSF10 gene.

The gene therapy DNA vector GDTT1.8NAS12-TNFSF10 was constructed by cloning the coding region of the TNFSF10 gene (849 bp) to a 2591 bp DNA vector GDTT1.8NAS12 by BamHI and HindIII restriction sites. The coding region of the TNFSF10 gene (849 bp) was obtained by isolating total RNA from the biological human tissue sample followed by reverse transcription reaction using commercial kit Mint-2 (Evrogen) and PCR amplification using the following oligonucleotides:

TNFSF10-F
AGGATCCACCATGGCTATGATGGAGGTCCA,
TNFSF10-R
TGTCGACTCAGCCAACTAAAAAGGCCCCGA,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 3402 bp DNA vector GDTT1.8NAS12-TNFSF10 with the nucleotide sequence SEQ ID No. 6 and general structure shown in FIG. 1F.

The gene therapy DNA vector GDTT1.8NAS12 was constructed as described in Example 1.

### Example 7

Production of the gene therapy DNA vector GDTT1.8NAS12-BCL2 carrying the therapeutic gene, namely the BCL2 gene.

The gene therapy DNA vector GDTT1.8NAS12-BCL2 was constructed by cloning the coding region of the BCL2 gene (724 bp) to a 2591 bp DNA vector GDTT1.8NAS12 by SalI and KpnI restriction sites. The coding region of the BCL2 gene (724 bp) was obtained by isolating total RNA from the biological human tissue sample followed by reverse transcription reaction using commercial kit Mint-2 (Evrogen) and PCR amplification using the following oligonucleotides:

BCL-F
ATCGTCGACCACCATGGCGCACGCTGGGAGA,
BCL-R
TTCGGTACCTCACTTGTGGCCCA,

and commercially available kit Phusion® High-Fidelity DNA Polymerase (New England Biolabs, USA); the amplification product and the DNA vector GDTT1.8NAS12 were cleaved by restriction endonucleases SalI and KpnI.

This resulted in a 3277 bp DNA vector GDTT1.8NAS12-BCL2 with the nucleotide sequence SEQ ID No. 7 and general structure shown in FIG. 1G.

The gene therapy DNA vector GDTT1.8NAS12 was constructed as described in Example 1.

### Example 8

Production of the gene therapy DNA vector GDTT1.8NAS12-HGF carrying the therapeutic gene, namely the HGF gene.

The gene therapy DNA vector GDTT1.8NAS12-HGF was constructed by cloning the coding region of the HGF gene (2190 bp) to a 2591 bp DNA vector GDTT1.8NAS12 by BamHI and SalI restriction sites. The coding region of the HGF gene (2190 bp) was obtained by isolating total RNA from the biological human tissue sample followed by reverse transcription reaction using commercial kit Mint-2 (Evrogen) and PCR amplification using the following oligonucleotides:

HGF_F
TTTGGATCCACCATGTGGGTGACCAAACTCCTGCCA,
HGF_R
AATGTCGACCTATGACTGTGGTACCTTATATGTTAAAAT,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 4775 bp DNA vector GDTT1.8NAS12-HGF with the nucleotide sequence SEQ ID No. 8 and general structure shown in FIG. 1H.

The gene therapy DNA vector GDTT1.8NAS12 was constructed as described in Example 1.

### Example 9

Production of the gene therapy DNA vector GDTT1.8NAS12-IL2 carrying the therapeutic gene, namely the IL-2 gene.

The gene therapy DNA vector GDTT1.8NAS12-IL2 was constructed by cloning the coding region of the IL-2 gene (465 bp) to a 2591 bp DNA vector GDTT1.8NAS12 by BamHI and SalI restriction sites. The coding region of the IL-2 gene (465 bp) was obtained by isolating total RNA from the biological human tissue sample followed by reverse transcription reaction using commercial kit Mint-2 (Evrogen) and PCR amplification using the following oligonucleotides:

IL-2-F
AGGATCCACCATGTACAGGATGCAACTCCT,
IL-2-R
TGTCGACTCAAGTCAGTGTTGAGATGATG,

and commercially available kit Phusion® High-Fidelity DNA Polymerase (New England Biolabs, USA); the amplification product and the DNA vector GDTT1.8NAS12 were cleaved by BamHI and SalI restriction endonucleases.

This resulted in a 3018 bp DNA vector GDTT1.8NAS12-IL2 with the nucleotide sequence SEQ ID No. 9 and general structure shown in FIG. 1I.

The gene therapy DNA vector GDTT1.8NAS12 was constructed as described in Example 1.

### Example 10

Proof of the ability of the gene therapy DNA vector GDTT1.8NAS12-DDC carrying the therapeutic gene, namely the DDC gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the DDC therapeutic gene were assessed in HCN-2 human cortical neuron cell line (ATCC CRL-10742) 48 hours after its transfection with the gene therapy DNA vector GDTT1.8NAS12-DDC carrying the human DDC gene. The amount of mRNA was determined by the dynamics of accumulation of cDNA amplicons in the real-time PCR.

The HCN-2 cell line was cultured under standard conditions (37° C., 5% CO2) using the DMEM growth medium with the addition of 10% fetal bovine serum (Gibco). The growth medium was replaced every 48 hours during the cultivation process.

To achieve 90% confluence, 24 hours before the transfection procedure, the cells were seeded into a 24-well plate in the quantity of 5×104 cells per well. Transfection with the gene therapy DNA vector GDTT1.8NAS12-DDC expressing the human DDC gene was performed using Lipofectamine 3000 (ThermoFisher Scientific, USA) according to the manufacturer's recommendations. In test tube 1, 1 μl of DNA vector GDTT1.8NAS12-DDC solution (in concentration of 500 ng/μl) and 1 μl of P3000 reagent was added to 25 μl of the Opti-MEM medium (Gibco, USA). The preparation was mixed by gentle shaking. In test tube 2, 1 μl of Lipofectamine 3000 solution was added to 25 μl of Opti-MEM medium (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

The HCN-2 cells transfected with the gene therapy DNA vector GDTT1.8NAS12 devoid of the inserted therapeutic gene (cDNA of the DDC gene before and after transfection with the gene therapy DNA vector GDTT1.8NAS12 devoid of the inserted therapeutic gene is not shown in the figures) were used as a reference. The reference vector GDTT1.8NAS12 for transfection was prepared as described above.

Total RNA was extracted from the HCN-2 cells using Trizol Reagent (Invitrogen, USA) according to the manufacturer's recommendations. 1 ml of Trizol Reagent was added to the well with cells, homogenised and heated for 5 minutes at 65° C. Then the sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The packed cells were rinsed in 1 ml of 70% ethyl alcohol, air-dried, and dissolved in 10 μl of RNase-free water. The level of DDC mRNA expression after transfection was determined by assessing the dynamics of the cDNA amplicon accumulation by real-time PCR. For production and amplification of cDNA specific for the human DDC gene, the following DDC_SF and DDC_SR oligonucleotides were used:

DDC_SF
GGAGAAGGGGGAGGAGTGAT,
DDC_SR
CCCAGCTCTTTCCACTGAGG.

The length of amplification product is 186 bp.

Reverse transcription reaction and PCR amplification was performed using SYBR GreenQuantitect RT-PCR Kit (Qiagen, USA) for real-time PCR. The reaction was carried out in a volume of 20 μl containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles of denaturation at 94° C. for 15 seconds, the annealing of primers at 60° C. for 30 seconds, and elongation at 72° C. for 30 seconds. The B2M (beta-2-microglobulin) gene listed in the GenBank database under number NM 004048.2 was used as a reference gene. Positive control included amplicons from PCR on matrices represented by plasmids in known concentrations containing cDNA sequences of the DDC and B2M genes. Deionised water was used as negative control. Real-time quantification of the dynamics of accumulation of cDNA amplicons of the DDC and B2M genes was conducted using the Bio-Rad CFX Manager 2.1 software (Bio-Rad, USA). Diagrams resulting from the assay are shown in FIG. 2.

FIG. 2 shows that the level of specific mRNA of the human DDC gene has grown massively as a result of transfection of the HCN-2 human cortical neuron cell line with the gene therapy DNA vector GDTT1.8NAS12-DDC, which confirms the ability of the vector to penetrate eukaryotic cells and express the DDC gene at the mRNA level. The presented results also confirm the practicability of use of the gene therapy DNA vector GDTT1.8NAS12-DDC in order to increase the expression level of the DDC gene in eukaryotic cells.

### Example 11

Proof of the ability of the gene therapy DNA vector GDTT1.8NAS12-IL10 carrying the therapeutic gene, namely the IL10 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the IL10 therapeutic gene were assessed in the IMR-32 human neuroblastoma cell line (ATCC CCL-127) 48 hours after its transfection with the gene therapy DNA vector GDTT1.8NAS12-IL10 carrying the human IL10 gene. The amount of mRNA was determined by the dynamics of accumulation of cDNA amplicons in the real-time PCR.

The IMR-32 cell line was cultured under standard conditions (37° C., 5% CO2) using the EMEM growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-IL10 expressing the human IL10 gene was performed according to the procedure described in Example 10. The B2M (beta-2-microglobulin) gene listed in the GenBank database under number NM 004048.2 was used as a reference gene. The IMR-32 human neuroblastoma cell line transfected with the gene therapy DNA vector GDTT1.8NAS12 devoid of the therapeutic gene (cDNA of the IL10 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human IL10 gene, the following IL10_SF and IL10_SR oligonucleotides were used:

IL10_SF
AAGACCCAGACATCAAGGCG,
IL10_SR
AGGCATTCTTCACCTGCTCC.

The length of amplification product is 135 bp.

Positive control included amplicons from PCR on matrices represented by plasmids in known concentrations containing cDNA sequences of the IL10 and B2M genes. Deionised water was used as negative control. Real-time quantification of the PCR products, i.e. the IL10 and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that the level of specific mRNA of the human IL10 gene has grown massively as a result of transfection of the IMR-32 human neuroblastoma cell line with the gene therapy DNA vector GDTT1.8NAS12-IL10, which confirms the ability of the vector to penetrate eukaryotic cells and express the IL10 gene at the mRNA level. The presented results also confirm the practicability of use of the gene therapy DNA vector GDTT1.8NAS12-IL10 in order to increase the expression level of the IL10 gene in eukaryotic cells.

### Example 12

Proof of the ability of the gene therapy DNA vector GDTT1.8NAS12-IL13 carrying the therapeutic gene, namely the IL13 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the IL13 therapeutic gene were assessed in the U-118 MG human glioblastoma cell line (ATCC HTB-15) 48 hours after its transfection with the gene therapy DNA vector GDTT1.8NAS12-IL13 carrying the human IL13 gene. The amount of mRNA was determined by the dynamics of accumulation of cDNA amplicons in the real-time PCR.

The U-118 MG cell line was cultured under standard conditions (37° C., 5% CO2) using the MEM growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-IL13 expressing the human IL13 gene was performed according to the procedure described in Example 10. The B2M (beta-2-microglobulin) gene listed in the GenBank database under number NM 004048.2 was used as a reference gene. The U-118 MG human glioblastoma cell line transfected with the gene therapy DNA vector GDTT1.8NAS12 devoid of the therapeutic gene (cDNA of the IL13 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human IL13 gene, the following IL13_SF and IL13_SR oligonucleotides were used:

IL13_SF
CATGGCGCTTTTGTTGACCA,
IL13_SR
AGCTGTCAGGTTGATGCTCC.

The length of amplification product is 181 bp.

Positive control included amplicons from PCR on matrices represented by plasmids in known concentrations containing cDNA sequences of the IL13 and B2M genes. Deionised water was used as negative control. Real-time quantification of the PCR products, i.e. the IL13 and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that the level of specific mRNA of the human IL13 gene has grown massively as a result of transfection of the U-118 MG human glioblastoma cell line with the gene therapy DNA vector GDTT1.8NAS12-IL13, which confirms the ability of the vector to penetrate eukaryotic cells and express the IL13 gene at the mRNA level. The presented results also confirm the practicability of use of the gene therapy DNA vector GDTT1.8NAS12-IL13 in order to increase the expression level of the IL13 gene in eukaryotic cells.

### Example 13

Proof of the ability of the gene therapy DNA vector GDTT1.8NAS12-IFNB1 carrying the therapeutic gene, namely the IFNB1 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the IFNB1 therapeutic gene were assessed in the SH-SY5Y human neuroblastoma cell line (ATCC CRL-2266) 48 hours after its transfection with the gene therapy DNA vector GDTT1.8NAS12-IFNB1 carrying the human IFNB1 gene. The amount of mRNA was determined by the dynamics of accumulation of cDNA amplicons in the real-time PCR.

The SH-SY5Y cell line was cultured under standard conditions (37° C., 5% CO2) using the 1:1 DMEM: F12 (Gibco) growth medium with the addition of 10% fetal bovine serum (Gibco). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. Transfection with the gene therapy DNA vector GDTT1.8NAS12-IFNB1 expressing the human IFNB1 gene was performed according to the procedure described in Example 10. The SH-SY5Y human neuroblastoma cell line transfected with the gene therapy DNA vector GDTT1.8NAS12 devoid of the therapeutic gene (cDNA of the IFNB1 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human IFNB1 gene, the following IFNB1_SF and IFNB1_SR oligonucleotides were used:

IFNB1_SF
GTGGCAATTGAATGGGAGGC,
IFNB1_SR
ATAGATGGTCAATGCGGCGT.

The length of amplification product is 118 bp.

Positive control included amplicons from PCR on matrices represented by plasmids in known concentrations containing cDNA sequences of the IFNB 1 and B2M genes. The B2M (beta-2-microglobulin) gene listed in the GenBank database under number NM 004048.2 was used as a reference gene. Deionised water was used as negative control. Real-time quantification of the PCR products, i.e. the IFNB1 and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that the level of specific mRNA of the human IFNB1 gene has grown massively as a result of transfection of the SH-SY5Y human neuroblastoma cell line with the gene therapy DNA vector GDTT1.8NAS12-IFNB1, which confirms the ability of the vector to penetrate eukaryotic cells and express the IFNB1 gene at the mRNA level. The results presented also confirm the practicability of use of the gene therapy DNA vector GDTT1.8NAS12-IFNB1 in order to increase the expression level of the IFNB1 gene in eukaryotic cells.

### Example 14

Proof of the ability of the gene therapy DNA vector GDTT1.8NAS12-TNFRSF4 carrying the therapeutic gene, namely the TNFRSF4 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the TNFRSF4 therapeutic gene were assessed in the SVG p12 human glial cell line (ATCC CRL-8621) 48 hours after its transfection with the gene therapy DNA vector GDTT1.8NAS12-TNFRSF4 carrying the human TNFRSF4 gene. The amount of mRNA was determined by the dynamics of accumulation of cDNA amplicons in the real-time PCR.

The SVG p12 cell line was cultured under standard conditions (37° C., 5% CO2) using the MEM growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-TNFRSF4 expressing the human TNFRSF4 gene was performed according to the procedure described in Example 10. The SVG p12 human glial cell line transfected with the gene therapy DNA vector GDTT1.8NAS12 devoid of the therapeutic gene (cDNA of the TNFRSF4 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human TNFRSF4 gene, the following TNFRSF4_SF and TNFRSF4 SR oligonucleotides were used:

TNFRSF4_SF
CTGGACAGCTACAAGCCTGG,
TNFRSF4_SR
CCTGTCCTCACAGATTGCGT.

The length of amplification product is 162 bp.

Positive control included amplicons from PCR on matrices represented by plasmids in known concentrations containing cDNA sequences of the TNFRSF4 and B2M genes. The B2M (beta-2-microglobulin) gene listed in the GenBank database under number NM 004048.2 was used as a reference gene. Deionised water was used as negative control. Real-time quantification of the PCR products, i.e. the TNFRSF4 and B2M gene cDNAs obtained by amplification, was conducted using the Bio-Rad CFX Manager 2.1 software (Bio-Rad, USA). Diagrams resulting from the assay are shown in FIG. 6.

FIG. 6 shows that the level of specific mRNA of the human TNFRSF4 gene has grown massively as a result of transfection of the SVG p12 human glial cell line with the gene therapy DNA vector GDTT1.8NAS12-TNFRSF4, which confirms the ability of the vector to penetrate eukaryotic cells and express the TNFRSF4 gene at the mRNA level. The results presented also confirm the practicability of use of the gene therapy DNA vector GDTT1.8NAS12-TNFRSF4 in order to increase the expression level of the TNFRSF4 gene in eukaryotic cells.

### Example 15

Proof of the ability of the gene therapy DNA vector GDTT1.8NAS12-TNFSF10 carrying the therapeutic gene, namely the TNFSF10 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the TNFSF10 therapeutic gene were assessed in the PFSK-1 human neuroectodermal tumour cell line (ATCC CRL-2060) 48 hours after its transfection with the gene therapy DNA vector GDTT1.8NAS12-TNFSF10 carrying the human TNFSF10 gene. The amount of mRNA was determined by the dynamics of accumulation of cDNA amplicons in the real-time PCR.

The PFSK-1 cell line was cultured under standard conditions (37° C., 5% CO2) using the RPMI-1640 growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-TNFSF10 expressing the human TNFSF10 gene was performed according to the procedure described in Example 10. The PFSK-1 human neuroectodermal tumour cell line transfected with the gene therapy DNA vector GDTT1.8NAS12 devoid of the therapeutic gene (cDNA of the TNFSF10 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human TNFSF10 gene, the following TNFSF10_SF and TNFSF10_SR oligonucleotides were used:

TNFSF10_SF
CCTGCAGTCTCTCTGTGTGG,
TNFSF10_SR
ACGGAGTTGCCACTTGACTT.

The length of amplification product is 181 bp.

Positive control included amplicons from PCR on matrices represented by plasmids in known concentrations containing cDNA sequences of the TNFSF10 and B2M genes. The B2M (beta-2-microglobulin) gene listed in the GenBank database under number NM 004048.2 was used as a reference gene. Deionised water was used as negative control. Real-time quantification of the PCR products, i.e. the TNFSF10 and B2M gene cDNAs obtained by amplification, was conducted using the Bio-Rad CFX Manager 2.1 software (Bio-Rad, USA). Diagrams resulting from the assay are shown in FIG. 7.

FIG. 7 shows that the level of specific mRNA of the human TNFSF10 gene has grown massively as a result of transfection of the PFSK-1 human neuroectodermal tumour cell line with the gene therapy DNA vector GDTT1.8NAS12-TNFSF10, which confirms the ability of the vector to penetrate eukaryotic cells and express the TNFSF10 gene at the mRNA level. The results presented also confirm the practicability of use of the gene therapy DNA vector GDTT1.8NAS12-TNFSF10 in order to increase the expression level of the TNFSF10 gene in eukaryotic cells.

### Example 16

Proof of the ability of the gene therapy DNA vector GDTT1.8NAS12-BCL2 carrying the therapeutic gene, namely the BCL2 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the BCL2 therapeutic gene were assessed in the D341 Med human medulloblastoma cell line (ATCC HTB-187) 48 hours after its transfection with the gene therapy DNA vector GDTT1.8NAS12-BCL2 carrying the human BCL2 gene. The amount of mRNA was determined by the dynamics of accumulation of cDNA amplicons in the real-time PCR.

The D341 Med cell line was cultured under standard conditions (37° C., 5% CO2) using the MEM growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-BCL2 expressing the human BCL2 gene was performed according to the procedure described in Example 10. The D341 Med human medulloblastoma cell line transfected with the gene therapy DNA vector GDTT1.8NAS12 devoid of the therapeutic gene (cDNA of the BCL2 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human BCL2 gene, the following BCL2_SF and BCL2_SR oligonucleotides were used:

BCL_SF
GAACTGGGGGAGGATTGTGG,
BCL_SR
CATCCCAGCCTCCGTTATCC.

The length of amplification product is 164 bp.

Positive control included amplicons from PCR on matrices represented by plasmids in known concentrations containing cDNA sequences of the BCL2 and B2M genes. The B2M (beta-2-microglobulin) gene listed in the GenBank database under number NM 004048.2 was used as a reference gene. Deionised water was used as negative control. Real-time quantification of the PCR products, i.e. the BCL2 and B2M gene cDNAs obtained by amplification, was conducted using the Bio-Rad CFX Manager 2.1 software (Bio-Rad, USA). Diagrams resulting from the assay are shown in FIG. 8.

FIG. 8 shows that the level of specific mRNA of the human BCL2 gene has grown massively as a result of transfection of the D341 Med human medulloblastoma cell line with the gene therapy DNA vector GDTT1.8NAS12-BCL2, which confirms the ability of the vector to penetrate eukaryotic cells and express the BCL2 gene at the mRNA level. The results presented also confirm the practicability of use of the gene therapy DNA vector GDTT1.8NAS12-BCL2 in order to increase the expression level of the BCL2 gene in eukaryotic cells.

### Example 17

Proof of the ability of the gene therapy DNA vector GDTT1.8NAS12-HGF carrying the therapeutic gene, namely the HGF gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the HGF therapeutic gene were assessed in the A-172 human brain astrocytoma cell culture (ATCC CRL-1620) 48 hours after its transfection with the gene therapy DNA vector GDTT1.8NAS12-HGF carrying the human HGF gene. The amount of mRNA was determined by the dynamics of accumulation of cDNA amplicons in the real-time PCR.

The A-172 cell culture was grown under standard conditions (37° C., 5% CO2) using the DMEM growth medium with the addition of 10% fetal bovine serum (Gibco). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-HGF expressing the human HGF gene was performed according to the procedure described in Example 10. The A-172 human brain astrocytoma cell culture transfected with the gene therapy DNA vector GDTT1.8NAS12 devoid of the therapeutic gene (cDNA of the HGF gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human HGF gene, the following HGF_SF and HGF_SR oligonucleotides were used:

HGF_SF
ACCCTGGTGTTTCACAAGCA,
HGF_FR
GCAAGAATTTGTGCCGGTGT.

The length of amplification product is 182 bp.

Positive control included amplicons from PCR on matrices represented by plasmids in known concentrations containing cDNA sequences of the HGF and B2M genes. The B2M (beta-2-microglobulin) gene listed in the GenBank database under number NM 004048.2 was used as a reference gene. Deionised water was used as negative control. Real-time quantification of the PCR products, i.e. the HGF and B2M gene cDNAs obtained by amplification, was conducted using the Bio-Rad CFX Manager 2.1 software (Bio-Rad, USA). Diagrams resulting from the assay are shown in FIG. 9.

FIG. 9 shows that the level of specific mRNA of the human HGF gene has grown massively as a result of transfection of the A-172 human brain astrocytoma cell culture with the gene therapy DNA vector GDTT1.8NAS12-HGF, which confirms the ability of the vector to penetrate eukaryotic cells and express the HGF gene at the mRNA level. The presented results also confirm the practicability of use of gene therapy DNA vector GDTT1.8NAS12-HGF in order to increase the expression level of the HGF gene in eukaryotic cells.

### Example 18

Proof of the ability of the gene therapy DNA vector GDTT1.8NAS12-IL2 carrying the therapeutic gene, namely the IL-2 gene, to penetrate eukaryotic cells and its functional activity at the level of therapeutic gene mRNA expression. This example also demonstrates practicability of the use of the gene therapy DNA vector carrying a therapeutic gene.

Changes in the mRNA accumulation of the IL-2 therapeutic gene were assessed in the ATCC-BXS0117 human neural progenitor cell culture (ATCC ACS-5003) 48 hours after their transfection with the gene therapy DNA vector GDTT1.8NAS12-IL2 carrying the human IL-2 gene. The amount of mRNA was determined by the dynamics of accumulation of cDNA amplicons in the real-time PCR.

The ATCC-BXS0117 cell line was grown under standard conditions (37° C., 5% CO2) using the 1:1 DMEM: F12 (Gibco) growth medium with the addition of 10% fetal bovine serum (Gibco) and the Growth Kit for Neural Progenitor Cell Expansion (ATCC ACS-3003). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. Transfection with the gene therapy DNA vector GDTT1.8NAS12-IL2 expressing the human IL-2 gene was performed according to the procedure described in Example 10. The ATCC-BXS0117 human neural progenitor cell line transfected with the gene therapy DNA vector GDTT1.8NAS12 devoid of the therapeutic gene (cDNA of the IL-2 gene before and after transfection with the gene therapy DNA vector GDTT1.8NAS12 devoid of the inserted therapeutic gene is not shown in the figures) was used as a reference. RNA isolation, reverse transcription reaction, and real-time PCR were performed as described in Example 10, except for oligonucleotides with sequences different from Example 10. For amplification of cDNA specific for the human IL-2 gene, the following IL-2_SF and IL-2_SR oligonucleotides were used:

IL-2_SF
ACAGGATGCAACTCCTGTCT,
IL-2_SR
GCATCCTGGTGAGTTTGGGA.

The length of amplification product is 192 bp.

Positive control included amplicons from PCR on matrices represented by plasmids in known concentrations containing cDNA sequences of the IL-2 and B2M genes. The B2M (beta-2-microglobulin) gene listed in the GenBank database under number NM 004048.2 was used as a reference gene. Deionised water was used as negative control. Real-time quantification of the PCR products, i.e. the IL-2 and B2M gene cDNAs obtained by amplification, was conducted using the Bio-Rad CFX Manager 2.1 software (Bio-Rad, USA). Diagrams resulting from the assay are shown in FIG. 10.

FIG. 10 shows that the level of specific mRNA of the human IL-2 gene has grown massively as a result of transfection of the ATCC-BXS0117 human neural progenitor cell line with the gene therapy DNA vector GDTT1.8NAS12-IL2, which confirms the ability of the vector to penetrate eukaryotic cells and express the IL-2 gene at the mRNA level. The results presented also confirm the practicability of use of the gene therapy DNA vector GDTT1.8NAS12-IL2 in order to increase the expression level of the IL-2 gene in eukaryotic cells.

### Example 19

Proof of the efficiency and practicability of use of the gene therapy DNA vector GDTT1.8NAS12-DDC carrying the DDC gene in order to increase expression of the DDC protein in mammalian cells.

Changes in the DDC protein concentration in the lysate of the HCN-2 human cortical neuron cell line (ATCC CRL-10742) were assessed after its transfection with the DNA vector GDTT1.8NAS12-DDC carrying the human DDC gene.

The HCN-2 human cortical neuron cell line was cultured as described in Example 10. To achieve 90% confluence, 24 hours before the transfection procedure, the cells were seeded into a 24-well plate in the quantity of 5×10⁴ cells per well. The 6th generation SuperFect Transfection Reagent (Qiagen, Germany) was used for transfection. The aqueous dendrimer solution without the DNA vector (A) and the DNA vector GDTT1.8NAS12 devoid of cDNA of the DDC gene (B) were used as a reference, and the DNA vector GDTT1.8NAS12-DDC carrying the human DDC gene (C) was used as a transfected agent. The DNA-dendrimer complex was prepared according to the manufacturer's procedure (QIAGEN, SuperFect Transfection Reagent Handbook, 2002) with certain modifications. For cell transfection in one well of a 24-well plate, the culture medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 ug/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 ug/ml of gentamicin was added and incubated for 24-48 hours at 37° C. in the presence of 5% CO2.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The DDC protein was assayed by enzyme-linked immunosorbent assay (ELISA) using the ELISA Kit for Dopa Decarboxylase (DDC) (SEG474Hu, Cloud-Clone Corp, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of the DDC protein was used. The sensitivity was at least 0.61 ng/ml, with measurement range from 1.56 ng/ml to 100 ng/ml. R-3.0.2 software (https://www.r-project.org/) was used for the statistical treatment of the results and data visualization. Diagrams resulting from the assay are shown in FIG. 11.

FIG. 11 shows that the transfection of the HCN-2 human cortical neuron cell line with the gene therapy DNA vector GDTT1.8NAS12-DDC results in increased DDC protein concentration compared to reference samples, which confirms the ability of the vector to penetrate eukaryotic cells and express the DDC gene at the protein level. The presented results also confirm the practicability of use of the gene therapy DNA vector GDTT1.8NAS12-DDC in order to increase the expression level of the DDC gene in eukaryotic cells.

### Example 20

Proof of the efficiency and practicability of use of the gene therapy DNA vector GDTT1.8NAS12-IL10 carrying the IL 10 gene in order to increase the expression of the IL10 protein in mammalian cells.

Changes in the protein concentration in the IMR-32 human neuroblastoma cell line (ATCC CCL-127) were assessed after its transfection with the DNA vector GDTT1.8NAS12-IL10 carrying the human IL10 gene. Cells were grown as described in Example 11.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the IL10 gene (B) were used as a reference, and the DNA vector GDTT1.8NAS12-IL10 carrying the human IL10 gene (C) was used as a transfected agent. Preparation of the DNA/dendrimer complex and transfection of the IMR-32 human neuroblastoma cell line were performed according to the procedure described in Example 19. After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The IL10 protein was assayed by enzyme-linked immunosorbent assay (ELISA) using the ELISA Kit for Interleukin 10 (IL10) (SEA056Hu, Cloud-Clone Corp, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of the IL10 protein was used. The sensitivity was at least 2.8 pg/ml, with the measurement range from 7.8 pg/ml to 500 pg/ml. R-3.0.2 software (https://www.r-project.org/) was used for the statistical treatment of the results and data visualization. Diagrams resulting from the assay are shown in FIG. 12.

FIG. 12 shows that the transfection of the IMR-32 human neuroblastoma cell line with the gene therapy DNA vector GDTT1.8NAS12-IL10 results in increased IL10 protein concentration compared to reference samples, which confirms the ability of the vector to penetrate eukaryotic cells and express the IL10 gene at the protein level. The presented results also confirm the practicability of use of the gene therapy DNA vector GDTT1.8NAS12-IL10 in order to increase the expression level of the IL 10 gene in eukaryotic cells.

### Example 21

Proof of the efficiency and practicability of use of the gene therapy DNA vector GDTT1.8NAS12-IL13 carrying the IL 13 gene in order to increase the expression of the IL13 protein in mammalian cells.

Changes in the IL13 protein concentration in the lysate of U-118 MG human glioblastoma cell (ATCC HTB-15) were assessed after its transfection with the DNA vector GDTT1.8NAS12-IL13 carrying the human IL13 gene. The cells were cultured as described in Example 12.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the IL13 gene (B) were used as a reference, and the DNA vector GDTT1.8NAS12-IL13 carrying the human IL13 gene (C) was used as a transfected agent. Preparation of the DNA/dendrimer complex and transfection of the U-118 MG human glioblastoma cell line were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The IL13 protein was assayed by enzyme-linked immunosorbent assay (ELISA) using the ELISA Kit for Interleukin 13 (IL13) (SEA060Hu, Cloud-Clone Corp, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of the IL13 protein was used. The sensitivity was at least 6.7 pg/ml, with the measurement range from 15.6 pg/ml to 1000 pg/ml. R-3.0.2 software (https://www.r-project.org/) was used for the statistical treatment of the results and data visualization. Diagrams resulting from the assay are shown in FIG. 13.

FIG. 13 shows that the transfection of the U-118 MG human glioblastoma cell line with the gene therapy DNA vector GDTT1.8NAS12-IL13 results in increased IL13 protein concentration compared to reference samples, which confirms the ability of the vector to penetrate eukaryotic cells and express the IL13 gene at the protein level. The presented results also confirm the practicability of use of the gene therapy DNA vector GDTT1.8NAS12-IL13 in order to increase the expression level of the IL13 gene in eukaryotic cells.

### Example 22

Proof of the efficiency and practicability of use of the gene therapy DNA vector GDTT1.8NAS12-IFNB1 carrying the IFNB1 gene in order to increase the expression of the IFNB 1 protein in mammalian cells.

Changes in the IFNB1 protein concentration in the lysate of the SH-SY5Y human neuroblastoma cell line (ATCC CRL-2266) were assessed after its transfection with the DNA vector GDTT1.8NAS12-IFNB1 carrying the human IFNB1 gene. The cells were cultured as described in Example 13.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the IFNB1 gene (B) were used as a reference, and the DNA vector GDTT1.8NAS12-IFNB1 carrying the human IFNB1 gene (C) was used as the transfected agent. Preparation of the DNA/dendrimer complex and transfection of the SH-SY5Y human neuroblastoma cell line were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The IFNB1 protein was assayed by enzyme-linked immunosorbent assay (ELISA) using the ELISA Kit for Interferon Beta (IFNb) (SEA222Hu, Cloud-Clone Corp, USA) according to the manufacturer's method with optical density detection using Chem Well Automated EIA and Chemistry Analyser (Awareness Technology Inc., USA).

To measure the numerical value of concentration, the calibration curve constructed using the reference samples from the kit with known concentrations of the IFNB1 protein was used. The sensitivity was at least 2.7 pg/ml, with the measurement range from 7.8 pg/ml to 500 pg/ml. R-3.0.2 software (https://www.r-project.org/) was used for the statistical treatment of the results and data visualization. Diagrams resulting from the assay are shown in FIG. 14.

FIG. 14 shows that the transfection of the SH-SY5Y human neuroblastoma cell line with the gene therapy DNA vector GDTT1.8NAS12-IFNB1 results in increased IFNB1 protein concentration compared to reference samples, which confirms the ability of the vector to penetrate eukaryotic cells and express the IFNB1 gene at the protein level. The results presented also confirm the practicability of use of the gene therapy DNA vector GDTT1.8NAS12-IFNB1 in order to increase the expression level of the IFNB1 gene in eukaryotic cells.

### Example 23

Proof of the efficiency and practicability of use of the gene therapy DNA vector GDTT1.8NAS12-TNFRSF4 carrying the TNFRSF4 gene in order to increase the expression of the TNFRSF4 protein in mammalian cells.

Changes in the TNFRSF4 protein concentration in the lysate of the SVG p12 human glial cell line (ATCC CRL-8621) were assessed after its transfection with the DNA vector GDTT1.8NAS12-TNFRSF4 carrying the human TNFRSF4 gene. The cells were cultured as described in Example 14.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the TNFRSF4 gene (B) were used as a reference, and the DNA vector GDTT1.8NAS12-TNFRSF4 carrying the human TNFRSF4 gene (C) was used as a transfected agent. Preparation of the DNA dendrimer complex and transfection of the SVG p12 human glial cells were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The TNFRSF4 protein was assayed by enzyme-linked immunosorbent assay (ELISA) using the Human OX40 ELISA Kit (ab231926, Abcam, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of the TNFRSF4 protein was used. The sensitivity was at least 23 pg/ml, with the measurement range from 46.9 pg/ml to 3000 pg/ml. R-3.0.2 software (https://www.r-project.org/) was used for the statistical treatment of the results and data visualization. Diagrams resulting from the assay are shown in FIG. 15.

FIG. 15 shows that the transfection of the SVG p12 glial cell line with the gene therapy DNA vector GDTT1.8NAS12-TNFRSF4 results in increased TNFRSF4 protein concentration compared to reference samples, which confirms the ability of the vector to penetrate eukaryotic cells and express the TNFRSF4 gene at the protein level. The results presented also confirm the practicability of use of the gene therapy DNA vector GDTT1.8NAS12-TNFRSF4 in order to increase the expression level of the TNFRSF4 gene in eukaryotic cells.

### Example 24

Proof of the efficiency and practicability of use of the gene therapy DNA vector GDTT1.8NAS12-TNFSF10 carrying the TNFSF10 gene in order to increase the expression of the TNFSF10 protein in mammalian cells.

Changes in the concentration of the TNFSF10 protein in the lysate of the PFSK-1 human neuroectodermal tumour cell line (ATCC CRL-2060) were assessed after transfection of the said cells with the gene therapy DNA vector GDTT1.8NAS12-TNFSF10 carrying the human TNFSF10 gene. The cells were cultured as described in Example 15.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the TNFSF10 gene (B) were used as a reference, and the DNA vector GDTT1.8NAS12-TNFSF10 carrying the human TNFSF10 gene (C) was used as a transfected agent. Preparation of the DNA dendrimer complex and transfection of the PFSK-1 human neuroectodermal tumour cells were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The TNFSF10 protein was assayed by enzyme-linked immunosorbent assay (ELISA) using the Human TRAIL/TNFSF10 Quantikine ELISA Kit (DTRL00, R&D Systems, Inc., USA) according to the manufacturer's method with optical density detection using Chem Well Automated EIA and Chemistry Analyser (Awareness Technology Inc., USA).

To measure the numerical value of concentration, the calibration curve constructed using the reference samples from the kit with known concentrations of the TNFSF10 protein was used. The sensitivity was at least 7.87 pg/ml, with the measurement range from 15.6 pg/ml to 1000 pg/ml. R-3.0.2 software (https://www.r-project.org/) was used for the statistical treatment of the results and data visualization. Diagrams resulting from the assay are shown in FIG. 16.

FIG. 16 shows that transfection of the PFSK-1 human neuroectodermal tumour cell line with the gene therapy DNA vector GDTT1.8NAS12-TNFSF10 results in increased TNFSF10 protein concentration compared to reference samples, which confirms the ability of the vector to penetrate eukaryotic cells and express the TNFSF10 gene at the protein level. The results presented also confirm the practicability of use of the gene therapy DNA vector GDTT1.8NAS12-TNFSF10 in order to increase the expression level of the TNFSF10 gene in eukaryotic cells.

### Example 25

Proof of the efficiency and practicability of use of the gene therapy DNA vector GDTT1.8NAS12-BCL2 carrying the BCL2 gene in order to increase the expression of the BCL2 protein in mammalian cells.

Changes in the BCL2 protein concentration in the lysate of the D341 Med human medulloblastoma cell culture (ATCC HTB-187) were assessed after its transfection with the DNA vector GDTT1.8NAS12-BCL2 carrying the human BCL2 gene. The cells were cultured as described in Example 16.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the BCL2 gene (B) were used as a reference, and the DNA vector GDTT1.8NAS12-BCL2 carrying the human BCL2 gene (C) was used as a transfected agent. Preparation of the DNA/dendrimer complex and transfection of the D341 Med human medulloblastoma cell line were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The BCL2 protein was assayed by enzyme-linked immunosorbent assay (ELISA) using the ELISA Kit for B-Cell Leukemia/Lymphoma 2 (Bcl2) (SEA778Hu, Cloud-Clone Corp., USA) according to the manufacturer's method with optical density detection using Chem Well Automated EIA and Chemistry Analyser (Awareness Technology Inc., USA).

To measure the numerical value of concentration, the calibration curve constructed using the reference samples from the kit with known concentrations of the BCL2 protein was used. The sensitivity was at least 0.061 ng/ml, with the measurement range from 0.156 ng/ml to 10 ng/ml. R-3.0.2 software (https://www.r-project.org/) was used for the statistical treatment of the results and data visualization. Diagrams resulting from the assay are shown in FIG. 17.

FIG. 17 shows that the transfection of the D341 Med human medulloblastoma cell line with the gene therapy DNA vector GDTT1.8NAS12-BCL2 results in increased BCL2 protein concentration compared to reference samples, which confirms the ability of the vector to penetrate eukaryotic cells and express the BCL2 gene at the protein level. The results presented also confirm the practicability of use of the gene therapy DNA vector GDTT1.8NAS12-BCL2 in order to increase the expression level of the BCL2 gene in eukaryotic cells.

### Example 26

Proof of the efficiency and practicability of use of the gene therapy DNA vector GDTT1.8NAS12-HGF carrying the HGF gene in order to increase the expression of the HGF protein in mammalian cells.

Changes in the HGF protein concentration in the lysate of the A-172 human brain astrocytoma cell culture (ATCC CRL-1620) were assessed after its transfection with the DNA vector GDTT1.8NAS12-HGF carrying the human HGF gene. The cells were cultured as described in Example 17.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the HGF gene (B) were used as a reference, and the DNA vector GDTT1.8NAS12-HGF carrying the human HGF gene (C) was used as a transfected agent. Preparation of the DNA dendrimer complex and transfection of the A-172 human brain astrocytoma cells were performed according to the procedure described in Example 19.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The HGF protein was assayed by enzyme-linked immunosorbent assay (ELISA) using the Human HGF ELISA Kit (ab 100534, Abcam, USA) according to the manufacturer's method with optical density detection using Chem Well Automated EIA and Chemistry Analyser (Awareness Technology Inc., USA).

To measure the numerical value of concentration, the calibration curve constructed using the reference samples from the kit with known concentrations of the HGF protein was used. The sensitivity was at least 3 pg/ml, with the measurement range from 2.74 pg/ml to 2000 pg/ml. R-3.0.2 software (https://www.r-project.org/) was used for the statistical treatment of the results and data visualization. Diagrams resulting from the assay are shown in FIG. 18.

FIG. 18 shows that transfection of the A-172 human brain astrocytoma cell culture with the gene therapy DNA vector GDTT1.8NAS12-HGF results in increased HGF protein concentration compared to reference samples, which confirms the ability of the vector to penetrate eukaryotic cells and express the HGF gene at the protein level. The presented results also confirm the practicability of use of the gene therapy DNA vector GDTT1.8NAS12-HGF in order to increase the expression level of the HGF gene in eukaryotic cells.

### Example 27

Proof of the efficiency and practicability of use of the gene therapy DNA vector GDTT1.8NAS12-IL2 carrying the IL-2 gene in order to increase the expression of the IL-2 protein in mammalian cells.

Changes in the IL-2 protein concentration in the lysate of the ATCC-BXS0117 human neural progenitor cell line (ATCC ACS-5003) were assessed after its transfection with the DNA vector GDTT1.8NAS12-IL2 carrying the human IL-2 gene. The cells were cultured as described in Example 18.

The 6th generation SuperFect Transfection Reagent (Qiagen, Germany) was used for transfection. The aqueous dendrimer solution without the DNA vector (A) and the DNA vector GDTT1.8NAS12 devoid of cDNA of the IL-2 gene (B) were used as a reference, and the DNA vector GDTT1.8NAS12-IL2 carrying the human IL-2 gene (C) was used as a transfected agent.

Preparation of the DNA/dendrimer complex and transfection of the ATCC-BXS0117 human neural progenitor cell line were performed according to the procedure described in Example 19. After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The IL-2 protein was assayed by enzyme-linked immunosorbent assay (ELISA) using the ELISA Kit for Interleukin 2 (IL-2) (SEA073Hu, Cloud-Clone Corp, USA) according to the manufacturer's method with optical density detection using Chem Well Automated EIA and Chemistry Analyser (Awareness Technology Inc., USA).

To measure the numerical value of concentration, the calibration curve constructed using the reference samples from the kit with known concentrations of IL-2 protein was used. The sensitivity was at least 5.9 pg/ml, with the measurement range from 15.6 pg/ml to 1000 pg/ml. R-3.0.2 software (https://www.r-project.org/) was used for the statistical treatment of the results and data visualization. Diagrams resulting from the assay are shown in FIG. 19.

FIG. 19 shows that the transfection of the ATCC-BXS0117 human neural progenitor cell culture with the gene therapy DNA vector GDTT1.8NAS12-IL2 results in increased IL-2 protein concentration compared to reference samples, which confirms the ability of the vector to penetrate eukaryotic cells and express the IL-2 gene at the protein level. The results presented also confirm the practicability of use of the gene therapy DNA vector GDTT1.8NAS12-IL2 in order to increase the expression level of the IL-2 gene in eukaryotic cells.

### Example 28

Proof of the efficiency and practicability of use of the gene therapy DNA vector GDTT1.8NAS12-IFNB1 carrying the IFNB1 gene in order to increase the expression of the IFNB 1 protein in human tissues.

To confirm the efficiency of the gene therapy DNA vector GDTT1.8NAS12-IFNB1 carrying the therapeutic gene, namely the IFNB1 gene, and practicability of its use, changes in the IFNB1 protein concentration in human skin upon intradermal injection of the gene therapy DNA vector GDTT1.8NAS12-IFNB1 carrying the human IFNB1 gene were assessed.

To analyse changes in the IFNB1 protein concentration, the gene therapy DNA vector GDTT1.8NAS12-IFNB1 carrying the IFNB1 gene was injected into the forearm skin of three patients with concurrent injection of a placebo being the gene therapy DNA vector GDTT1.8NAS12 devoid of cDNA of the IFNB1 gene.

Patient 1, female, 48 y.o. (P1); patient 2, female, 66 y.o. (P2); patient 3, male, 44 y.o. (P3). Polyethylenimine Transfection reagent cGMP grade in-vivo-jetPEI (Polyplus Transfection, France) was used as a transport system. The gene therapy DNA vector GDTT1.8NAS12-IFNB1 containing cDNA of the IFNB1 gene and the gene therapy DNA vector GDTT1.8NAS12 used as a placebo not containing cDNA of the IFNB1 gene were dissolved in sterile nuclease-free water. To obtain a gene construct, DNA-cGMP grade in-vivo-jetPEI complexes were prepared according to the manufacturer recommendations.

The gene therapy DNA vector GDTT1.8NAS12 (placebo) and the gene therapy DNA vector GDTT1.8NAS12-IFNB1 carrying the IFNB1 gene were injected in the quantity of 1 mg of each genetic construct using the tunnel method with a 30G needle to the depth of 3 mm. The injectate volume of the gene therapy DNA vector GDTT1.8NAS12 (placebo) and the gene therapy DNA vector GDTT1.8NAS12-IFNB1 carrying the IFNB1 gene was 0.5 ml for each genetic construct. The points of injection of each genetic construct were located at 8 to 10 cm intervals at the forearm site.

Biopsy samples were taken on the 2nd day after the injection of the genetic constructs of the gene therapy DNA vectors. The biopsy samples were taken from the patients' skin in the site of injection of the gene therapy DNA vector GDTT1.8NAS12-IFNB1 carrying the IFNB1 gene (I), the gene therapy DNA vector GDTT1.8NAS12 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample had a size of ca. 10 mm3 and the weight was approximately 11 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used to assay the therapeutic protein by enzyme-linked immunosorbent assay (ELISA). The IFNB1 protein was assayed by enzyme-linked immunosorbent assay (ELISA) as described in Example 22.

To measure the numerical value of concentration, the calibration curve constructed using the reference samples from the kit with known concentrations of the IFNB1 protein was used. R-3.0.2 software (https://www.r-project.org/) was used for the statistical treatment of the results and data visualization according to the manufacturer's method with optical density detection using Chem Well Automated EIA and Chemistry Analyser (Awareness Technology Inc., USA). Diagrams resulting from the assay are shown in FIG. 20.

FIG. 20 shows the increased IFNB1 protein concentration in the skin of all three patients in the injection site of the gene therapy DNA vector GDTT1.8NAS12-IFNB 1 carrying the human IFNB 1 therapeutic gene compared to the IFNB1 protein concentration in the injection site of the gene therapy DNA vector GDTT1.8NAS12 (placebo) devoid of the human IFNB1 gene, which indicates the efficiency of the gene therapy DNA vector GDTT1.8NAS12-IFNB1 and confirms the practicability of its use, in particular upon intradermal injection of the gene therapy DNA vector in human tissues.

### Example 29

Proof of the efficiency and practicability of use of the gene therapy DNA vector GDTT1.8NAS12-IL13 carrying the IL 13 gene in order to increase the expression of the IL13 protein in human tissues.

To confirm the efficiency of the gene therapy DNA vector GDTT1.8NAS12-IL 13 carrying the IL13 therapeutic gene and practicability of its use, the change in the IL13 protein concentration in human muscle tissues upon injection of the gene therapy DNA vector GDTT1.8NAS12-IL13 carrying the therapeutic gene, namely the human IL13 gene, was assessed.

To analyse changes in the concentration of the IL13 protein, the gene therapy DNA vector GDTT1.8NAS12-IL13 carrying the IL 13 gene with transport molecule was injected into the gastrocnemius muscle of three patients with concurrent injection of a placebo being the gene therapy DNA vector GDTT1.8NAS12 devoid of cDNA of the IL13 gene with transport molecule.

Patient 1, female, 49 y.o. (P1); patient 2, male, 54 y.o. (P2); patient 3, male, 53 y.o. (P3). Polyethylenimine Transfection reagent cGMP grade in-vivo-jetPEI (Polyplus Transfection, France) was used as a transport system; sample preparation was carried out in accordance with the manufacturer's recommendations.

The gene therapy DNA vector GDTT1.8NAS12 (placebo) and the gene therapy DNA vector GDTT1.8NAS12-IL13 carrying the IL13 gene were injected in the quantity of 1 mg for each genetic construct using the tunnel method with a 30G needle to the depth of around 10-12 mm. The injectate volume of the gene therapy DNA vector GDTT1.8NAS12 (placebo) and the gene therapy DNA vector GDTT1.8NAS12-IL13 carrying the IL13 gene was 1 ml for each genetic construct. The points of injection of each genetic construct were located medially at 8 to 10 cm intervals.

Biopsy samples were taken on the 2nd day after the injection of the genetic constructs of the gene therapy DNA vectors. The biopsy samples were taken from the patients' muscle tissues in the site of injection of the gene therapy DNA vector GDTT1.8NAS12-IL 13 carrying the IL13 gene (I), the gene therapy DNA vector GDTT1.8NAS12 (placebo) (II), and intact site of gastrocnemius muscle (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used to assay the therapeutic protein.

The IL13 protein was assayed by enzyme-linked immunosorbent assay (ELISA) as described in Example 21.

To measure the numerical value of concentration, the calibration curve constructed using the reference samples from the kit with known concentrations of the IL13 protein was used. R-3.0.2 software (https://www.r-project.org/) was used for the statistical treatment of the results and data visualization. Diagrams resulting from the assay are shown in FIG. 21.

FIG. 21 shows the increased IL13 protein concentration in the gastrocnemius muscle of all three patients in the injection site of the gene therapy DNA vector GDTT1.8NAS12-IL13 carrying the therapeutic gene, namely the human IL13 gene, compared to the IL13 protein concentration in the injection site of the gene therapy DNA vector GDTT1.8NAS12 (placebo) devoid of the human IL13 gene, which indicates the efficiency of the gene therapy DNA vector GDTT1.8NAS12-IL13 and confirms the practicability of its use, in particular upon intramuscular injection of the gene therapy DNA vector in human tissues.

### Example 30

Proof of the efficiency and practicability of use of the gene therapy DNA vector GDTT1.8NAS12-IL2 carrying the IL-2 gene in order to increase the expression of the IL-2 protein in human tissues.

To confirm the efficiency of the gene therapy DNA vector GDTT1.8NAS12-IL2 carrying the therapeutic gene, namely the IL-2 gene, and practicability of its use, changes in the IL-2 protein concentration in human skin upon intradermal injection of the gene therapy DNA vector GDTT1.8NAS12-IL2 carrying the human IL-2 gene were assessed.

To analyse changes in the IL-2 protein concentration, the gene therapy DNA vector GDTT1.8NAS12-IL2 carrying the IL-2 gene was injected into the forearm skin of three patients with concurrent injection of a placebo being the gene therapy DNA vector GDTT1.8NAS12 devoid of cDNA of the IL-2 gene.

Patient 1, female, 52 y.o. (P1); patient 2, male, 39 y.o. (P2); patient 3, male, 40 y.o. (P3). Polyethylenimine Transfection reagent cGMP grade in-vivo-jetPEI (Polyplus Transfection, France) was used as a transport system. The gene therapy DNA vector GDTT1.8NAS12-IL2 containing cDNA of the IL-2 gene and the gene therapy DNA vector GDTT1.8NAS12 used as a placebo not containing cDNA of the IL-2 gene were dissolved in sterile nuclease-free water. To obtain a gene construct, DNA-cGMP grade in-vivo-jetPEI complexes were prepared according to the manufacturer recommendations.

The gene therapy DNA vector GDTT1.8NAS12 (placebo) and the gene therapy DNA vector GDTT1.8NAS12-IL2 carrying the IL-2 gene were injected in the quantity of 1 mg of each genetic construct using the tunnel method with a 30G needle to the depth of 3 mm. The injectate volume of the gene therapy DNA vector GDTT1.8NAS12 (placebo) and the gene therapy DNA vector GDTT1.8NAS12-IL2 carrying the IL-2 gene was 0.5 ml for each genetic construct. The points of injection of each genetic construct were located at 8 to 10 cm intervals at the forearm site.

Biopsy samples were taken on the 2nd day after the injection of the genetic constructs of the gene therapy DNA vectors. The biopsy samples were taken from the patients' skin in the site of injection of the gene therapy DNA vector GDTT1.8NAS12-IL2 carrying the IL-2 gene (I), the gene therapy DNA vector GDTT1.8NAS12 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample had a size of ca. 10 mm3 and the weight was approximately 11 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used to assay the therapeutic protein by enzyme-linked immunosorbent assay (ELISA) as described in Example 27.

To measure the numerical value of concentration, the calibration curve constructed using the reference samples from the kit with known concentrations of IL-2 protein was used. R-3.0.2 software (https://www.r-project.org/) was used for the statistical treatment of the results and data visualization. Diagrams resulting from the assay are shown in FIG. 22.

FIG. 22 shows the increased IL-2 protein concentration in the skin of all three patients in the injection site of the gene therapy DNA vector GDTT1.8NAS12-IL2 carrying the human IL-2 therapeutic gene compared to the IL-2 protein concentration in the injection site of the gene therapy DNA vector GDTT1.8NAS12 (placebo) devoid of the human IL-2 gene, which indicates the efficiency of the gene therapy DNA vector GDTT1.8NAS12-IL2 and confirms the practicability of its use, in particular upon intradermal injection of the gene therapy DNA vector in human tissues.

### Example 31

Proof of the efficiency of the gene therapy DNA vector GDTT1.8NAS12-IL10 carrying the IL10 gene and practicability of its use in order to increase the expression level of the IL10 protein in human tissues by injecting autologous fibroblasts transfected with the gene therapy DNA vector GDTT1.8NAS12-IL10.

To confirm the efficiency of the gene therapy DNA vector GDTT1.8NAS12-IL10 carrying the IL10 gene and practicability of its use, changes in the IL10 protein concentration in human skin were assessed upon injection of patient's skin with autologous fibroblast culture of the same patient transfected with the gene therapy DNA vector GDTT1.8NAS12-IL10.

The appropriate autologous fibroblast culture transfected with the gene therapy DNA vector GDTT1.8NAS12-IL10 carrying the IL 10 gene was injected into the patient's forearm skin with concurrent injection of a placebo in the form of autologous fibroblast culture transfected with the gene therapy DNA vector GDTT1.8NAS12 devoid of the IL10 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample size was ca. 10 mm, its weight was ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×10⁴ cells was taken from the cell culture. The patient's fibroblast culture was transfected with the gene therapy DNA vector GDTT1.8NAS12-IL10 carrying the IL10 gene or placebo, i.e. the GDTT1.8NAS12 vector devoid of the IL 10 therapeutic gene.

The transfection was carried out using a cationic polymer, polyethylenimine JETPEI (Polyplus Transfection, France), according to the manufacturer's instructions. The cells were cultured for 72 hours and then injected into the patient. Injection of autologous fibroblast culture of the patient transfected with the gene therapy DNA vector GDTT1.8NAS12-IL10 and autologous fibroblast culture of the patient transfected with the gene therapy DNA vector GDTT1.8NAS12 as a placebo was performed in the forearm using the tunnel method with a 13 mm long 30G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after injection of autologous fibroblast culture transfected with the gene therapy DNA vector GDTT1.8NAS12-IL10 carrying the therapeutic gene, namely the IL10 gene, and placebo. The biopsy samples were taken from the patients' skin in the site of injection of autologous fibroblast culture transfected with the gene therapy DNA vector GDTT1.8NAS12-IL10 carrying the therapeutic gene, namely the IL 10 gene (C), autologous fibroblast culture transfected with the gene therapy DNA vector GDTT1.8NAS12 not carrying the IL10 therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample had a size of ca. 10 mm3 and the weight was approximately 11 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used to assay the therapeutic protein as described in Example 12.

Diagrams resulting from the assay are shown in FIG. 23.

FIG. 23 shows the increased concentration of the IL10 protein in the area of the patient's skin in the injection site of autologous fibroblast culture transfected with the gene therapy DNA vector GDTT1.8NAS12-IL10 carrying the IL10 gene compared to the IL 10 protein concentration in the injection site of autologous fibroblast culture transfected with the gene therapy DNA vector GDTT1.8NAS12 devoid of the IL10 gene (placebo), which indicates the efficiency of the gene therapy DNA vector GDTT1.8NAS12-IL10 and practicability of its use in order to increase the expression level of the IL10 in human tissues, in particular upon injection of autologous fibroblasts transfected with the gene therapy DNA vector GDTT1.8NAS12-IL10 into the skin.

### Example 32

Proof of the efficiency and practicability of use of the constructed gene therapy DNA vector GDTT1.8NAS12-DDC carrying the therapeutic gene, namely the DDC gene, the gene therapy DNA vector GDTT1.8NAS12-BCL2 carrying the therapeutic gene, namely the BCL2 gene, the gene therapy DNA vector GDTT1.8NAS12-HGF carrying the therapeutic gene, namely the HGF gene, the gene therapy DNA vector GDTT1.8NAS12-TNFSF10 carrying the therapeutic gene, namely the TNFSF10 gene, the gene therapy DNA vector GDTT1.8NAS12-TNFRSF4 carrying the therapeutic gene, namely the TNFRSF4 gene, in order to increase the expression level of the DDC, BCL2, HGF, TNFSF10, and TNFRSF4 proteins in mammalian tissues.

Changes in concentrations of the DDC, BCL2, HGF, TNFSF10, and TNFRSF4 proteins in muscle tissue of rats were assessed upon joint injection of a mixture of the gene therapy vectors: the gene therapy DNA vector GDTT1.8NAS12-DDC, the gene therapy DNA vector GDTT1.8NAS12-BCL2, the gene therapy DNA vector GDTT1.8NAS12-HGF, the gene therapy DNA vector GDTT1.8NAS12-TNFSF10, and the gene therapy DNA vector GDTT1.8NAS12-TNFRSF4. The study involved 15 laboratory animals, namely male Wistar rats at 8 months of age weighing 240-290 g, divided into three groups: group K1 (5 animals) injected with the mixture of five gene therapy DNA vectors carrying the DDC, BCL2, HGF, TNFSF10, and TNFRSF4 genes; group K2 (5 animals) injected with the gene therapy DNA vector GDTT1.8NAS12 (placebo); and group K3 (5 animals) of intact animals. Polyethylenimine Transfection reagent cGMP grade in-vivo-jetPEI (Polyplus Transfection, France) was used as a transport system. Equimolar mixture of the gene therapy DNA vectors GDTT1.8NAS12-DDC, GDTT1.8NAS12-BCL2, GDTT1.8NAS12-HGF, GDTT1.8NAS12-TNFSF10, GDTT1.8NAS12-TNFRSF4, 30 ug of each, was dissolved in sterile nuclease-free water. To obtain a gene construct, DNA-cGMP grade in-vivo-jetPEI complexes were prepared according to the manufacturer recommendations.

The volume of solution for intramuscular injection was 0.15 ml with a total of 150 ug of DNA. The solution was injected using an insulin syringe. Rats were decapitated 2 days after the procedure.

The samples were taken on the 2nd day after the injection of the gene therapy DNA vectors. Biopsy material was taken from the right hip muscle of all animals, in the injection site of a mixture of the five gene therapy DNA vectors carrying the DDC, BCL2, HGF, TNFSF10, and TNFRSF4 genes (group 1), in the injection site of the placebo (group 2), and from the right hip muscle of intact animals (group 3). Each sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension obtained was then centrifuged for 10 minutes at 14,000 g. Supernatant was collected and used to assay the therapeutic proteins as described in Example 19 (quantification of the DDC protein), Example 25 (quantification of the BCL2 protein), Example 26 (quantification of the HGF protein), Example 24 (quantification of the TNFSF10 protein), and Example 23 (quantification of the TNFRSF4 protein). Diagrams resulting from the assay are shown in FIG. 24.

FIG. 24 shows an increase in the concentration of the DDC, BCL2, HGF, TNFSF10, and TNFRSF4 proteins in the muscle tissues of group K1 animals injected with the mixture of the gene therapy DNA vectors: the gene therapy DNA vector GDTT1.8NAS12-DDC carrying the DDC therapeutic gene, the gene therapy DNA vector GDTT1.8NAS12-BCL2 carrying the BCL2 therapeutic gene, the gene therapy DNA vector GDTT1.8NAS12-HGF carrying the HGF therapeutic gene, the gene therapy DNA vector GDTT1.8NAS12-TNFSF10 carrying the TNFSF10 therapeutic gene, and the gene therapy DNA vector GDTT1.8NAS12-TNFRSF4 carrying the TNFRSF4 therapeutic gene, compared to muscle tissues of the group K2 animals (placebo) and group K3 animals (intact group). The obtained results show the efficiency of combined use of gene therapy DNA vectors and practicability of use for the increase of the expression level of therapeutic proteins in mammalian tissues.

### Example 33

Proof of the efficiency of the gene therapy DNA vector GDTT1.8NAS12-IL13 carrying the IL13 gene and practicability of its use in order to increase the expression level of the IL13 protein in mammalian cells.

To confirm the efficiency of the gene therapy DNA vector GDTT1.8NAS12-IL13 carrying the IL13 gene, changes in mRNA accumulation of the IL13 therapeutic gene in bovine peripheral blood mononuclear cells 48 hours after their transfection with the gene therapy DNA vector GDTT1.8NAS12-IL13 carrying the human IL13 gene were assessed.

Bovine peripheral blood mononuclear cells were extracted from 50 ml of venous blood by centrifuging in the gradient of ficoll solution 1.077 (Paneco, P052p) and cultured in RPMI Media 1640 (GIBCOR, 11875-085) with the addition of 10% of horse serum (ATCC® 30-2040™) under standard conditions. Transfection with the gene therapy DNA vector GDTT1.8NAS12-IL13 carrying the human IL13 gene and the DNA vector GDTT1.8NAS12 devoid of the human IL13 gene (reference), RNA extraction, reverse transcription reaction, PCR amplification, and data analysis were performed as described in Example 12. Bull/cow actin gene (ACT) listed in the GenBank database under number AH001130.2 was used as a reference gene. Positive control included amplicons from PCR on matrices represented by plasmids in known concentrations containing the IL13 and ACT gene sequences. Deionised water was used as negative control. Real-time quantification of the PCR products, i.e. the IL13 and ACT gene cDNAs obtained by amplification, was conducted using the Bio-Rad CFX Manager 2.1 software (Bio-Rad, USA).

Diagrams resulting from the assay are shown in FIG. 25.

FIG. 25 shows that the level of specific mRNA of the human IL13 gene has grown massively as a result of transfection of the bovine peripheral blood mononuclear cells with the gene therapy DNA vector GDTT1.8NAS12-IL13, which confirms the ability of the vector to penetrate eukaryotic cells and express the IL 13 gene at the mRNA level. The presented results confirm the practicability of use of the gene therapy DNA vector GDTT1.8NAS12-IL13 in order to increase the expression level of the IL13 gene in mammalian cells.

### Example 34

Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.8NAS12-IFNB1, or Escherichia coli strain JM110-NAS/GDTT1.8NAS12-TNFRSF4, or Escherichia coli strain JM110-NAS/GDTT1.8NAS12-TNFSF10, or Escherichia coli strain JM110-NAS/GDTT1.8NAS12-BCL2, or Escherichia coli strain JM110-NAS/GDTT1.8NAS12-HGF, or Escherichia coli strain JM110-NAS/GDTT1.8NAS12-IL2, carrying the gene therapy DNA vector, and method of production thereof.

The strain construction for the production of the gene therapy DNA vector based on the gene therapy DNA vector GDTT1.8NAS12 carrying a therapeutic gene from the following group of genes: DDC, IL10, IL13, IFNB1, TNFRSF4, TNFSF10, BCL2, HGF, and IL-2, namely the Escherichia coli strain JM110-NAS/GDTT1.8NAS12-DDC, or Escherichia coli strain JM110-NAS/GDTT1.8NAS12-IL10, or Escherichia coli strain JM110-NAS/GDTT1.8NAS12-IL13, or Escherichia coli strain JM110-NAS/GDTT1.8NAS12-IFNB1, or Escherichia coli strain JM110-NAS/GDTT1.8NAS12-TNFRSF4, or Escherichia coli strain JM110-NAS/GDTT1.8NAS12-TNFSF10, or Escherichia coli strain JM110-NAS/GDTT1.8NAS12-BCL2, or Escherichia coli strain JM110-NAS/GDTT1.8NAS12-HGF, or Escherichia coli strain JM110-NAS/GDTT1.8NAS12-IL2 carrying the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, respectively, for its production allowing for antibiotic-free selection involves obtaining electrocompetent cells of the Escherichia coli strain JM110-NAS and subjecting these cells to electroporation with the gene therapy DNA vector GDTT1.8NAS12-DDC, or the gene therapy DNA vector GDTT1.8NAS12-IL10, or the gene therapy DNA vector GDTT1.8NAS12-IL13, or GDTT1.8NAS12-IFNB1, or the gene therapy DNA vector GDTT1.8NAS12-TNFRSF4, or the gene therapy DNA vector GDTT1.8NAS12-TNFSF10, or the gene therapy DNA vector GDTT1.8NAS12-BCL2, or the gene therapy DNA vector GDTT1.8NAS12-HGF, or the gene therapy DNA vector GDTT1.8NAS12-IL2. After that, the cells were seeded into agar plates (Petri dishes) with a selective medium containing yeastrel, peptone, 6% sucrose, and 10 ug/ml of chloramphenicol. At the same time, production of the Escherichia coli strain JM110-NAS for the production of the gene therapy DNA vector GDTT1.8NAS12 or the gene therapy DNA vectors based on it allowing for antibiotic-free positive selection involves constructing a 64 bp linear DNA fragment that contains regulatory element RNA-IN of Tn10 transposon allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 ug/ml of chloramphenicol are selected.

The obtained strains for production were included in the collection of the National Biological Resource Centre-Russian National Collection of Industrial Microorganisms (NBRC RNCIM), RF and NCIMB Patent Deposit Service, UK under the following registration numbers:
the Escherichia coli strain JM110-NAS/GDTT1.8NAS12-DDC under RCIM Reg. No. B-______, deposited on 14 Jan. 2020; INTERNATIONAL DEPOSITARY AUTHORITY No. NCIMB 43543, deposited on 6 Jan. 2020,
the Escherichia coli strain JM110-NAS/GDTT1.8NAS12-IL10 under RCIM Reg. No. B-______, deposited on 14 Jan. 2020; INTERNATIONAL DEPOSITARY AUTHORITY No. NCIMB 43540, deposited on 6 Jan. 2020,
the Escherichia coli strain JM110-NAS/GDTT1.8NAS12-IL13 under RCIM Reg. No. B-______, deposited on 14 Jan. 2020; INTERNATIONAL DEPOSITARY AUTHORITY No. NCIMB 43542, deposited on 6 Jan. 2020,
the Escherichia coli strain JM110-NAS/GDTT1.8NAS12-IFNB1 under Reg. No. B-13534, deposited on 27 Nov. 2019; INTERNATIONAL DEPOSITARY AUTHORITY No. NCIMB 43525, deposited on 28Nov. 2019,
the Escherichia coli strain JM110-NAS/GDTT1.8NAS12-TNFRSF4 under RCIM Reg. No. B-______, deposited on 14 Jan. 2020; INTERNATIONAL DEPOSITARY AUTHORITY No. NCIMB 43545, deposited on 6 Jan. 2020,
the Escherichia coli strain JM110-NAS/GDTT1.8NAS12-TNFSF10 under RCIM Reg. No. B-______, deposited on 14 Jan. 2020; INTERNATIONAL DEPOSITARY AUTHORITY No. NCIMB 43539, deposited on 6 Jan. 2020,
the Escherichia coli strain JM110-NAS/GDTT1.8NAS12-BCL2 under RCIM Reg. No. B-______, deposited on 14 Jan. 2020; INTERNATIONAL DEPOSITARY AUTHORITY No. NCIMB 43546, deposited on 6 Jan. 2020,
the Escherichia coli strain JM110-NAS/GDTT1.8NAS12-HGF under RCIM Reg. No. B-______, deposited on 14 Jan. 2020; INTERNATIONAL DEPOSITARY AUTHORITY No. NCIMB 43541, deposited on 6 Jan. 2020,
the Escherichia coli strain JM110-NAS/GDTT1.8NAS12-IL2 under RCIM Reg. No. B-______, deposited on 14 Jan. 2020; INTERNATIONAL DEPOSITARY AUTHORITY No. NCIMB 43544, deposited on 6 Jan. 2020.

### Example 35

The method for scaling up of the gene therapy DNA vector based on the gene therapy DNA vector GDTT1.8NAS12 carrying the therapeutic gene selected from the group of DDC, IL10, IL13, IFNB1, TNFRSF4, TNFSF10, BCL2, HGF, IL-2, to a commercial scale.

To confirm the producibility and constructability and scale up of the production of the gene therapy DNA vector GDTT1.8NAS12-DDC (SEQ ID No. 1), or the gene therapy DNA vector GDTT1.8NAS12-IL10 (SEQ ID No. 2), or the gene therapy DNA vector GDTT1.8NAS12-IL13 (SEQ ID No. 3), or the gene therapy DNA vector GDTT1.8NAS12-IFNB1 (SEQ ID No. 4), or the gene therapy DNA vector GDTT1.8NAS12-TNFRSF4 (SEQ ID No. 5), or the gene therapy DNA vector GDTT1.8NAS12-TNFSF10 (SEQ ID No. 6), or the gene therapy DNA vector GDTT1.8NAS12-BCL2 (SEQ ID No. 7), or the gene therapy DNA vector GDTT1.8NAS12-HGF (SEQ ID No. 8), or the gene therapy DNA vector GDTT1.8NAS12-IL2 (SEQ ID No. 9), to an industrial scale, the industrial-scale fermentation the Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2, each containing the gene therapy DNA vector GDTT1.8NAS12 carrying the therapeutic gene, namely DDC, or IL10, or IL13, or IFNB1, or TNFRSF4, or TNFSF10, or BCL2, or HGF, or IL-2 gene, was performed. Each Escherichia coli strain JM110-NAS/GDTT1.8NAS12-DDC, or the Escherichia coli strain JM110-NAS/GDTT1.8NAS12-IL10, or the Escherichia coli strain JM110-NAS/GDTT1.8NAS12-IL13, or the Escherichia coli strain JM110-NAS/GDTT1.8NAS12-IFNB1, or the Escherichia coli strain JM110-NAS/GDTT1.8NAS12-TNFRSF4, or the Escherichia coli strain JM110-NAS/GDTT1.8NAS12-TNFSF10, or the Escherichia coli strain JM110-NAS/GDTT1.8NAS12-BCL2, or the Escherichia coli strain JM110-NAS/GDTT1.8NAS12-HGF, or the Escherichia coli strain JM110-NAS/GDTT1.8NAS12-IL2 was produced on the basis of the Escherichia coli strain JM110-NAS (Genetic Diagnostics and Therapy 21 Ltd, United Kingdom) as described in Example 34, by electroporation of competent cells of this strain with the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, carrying the therapeutic gene, namely DDC, or IL10, or IL13, or IFNB1, or TNFRSF4, or TNFSF10, or BCL2, or HGF, or IL-2 with further inoculation of transformed cells into agar plates (Petri dishes) with a selective medium containing yeastrel, peptone, and 6% sucrose, and selection of individual clones.

Fermentation of the resulting Escherichia coli strain JM110-NAS/GDTT1.8NAS12-DDC carrying the gene therapy DNA vector GDTT1.8NAS12-DDC was performed in a 10 L fermenter with subsequent extraction of the gene therapy DNA vector GDTT1.8NAS12-DDC.

For fermentation of the Escherichia coli strain JM110-NAS/GDTT1.8NAS12-DDC, medium containing the following ingredients per 10 L of volume was prepared: 100 g of tryptone and 50g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of the Escherichia coli strain JM110-NAS/GDTT1.8NAS12-DDC was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were precipitated by centrifugation for 30 minutes at 5,000-10,000 g. Supernatant was removed, and the cell precipitate was re-suspended in 10% (v/v) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell precipitate in the volume of 1000 ml, and the mixture was stirred thoroughly to a homogenised suspension. Then egg lysozyme solution was added to the final concentration of 100 ug/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then RNase A (Sigma, USA) was added to the final concentration of 20 ug/ml, and the solution was incubated overnight for 16 hours at room temperature. The solution was then centrifuged for 20-30 minutes at 15,000 g and passed through a 0.45 μm membrane filter (Millipore, USA). Then ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution, and then the gene therapy DNA vector GDTT1.8NAS12-DDC was eluted using a linear gradient of 25 mM Tris-HCl, pH 7.0, to obtain a solution of 25 mM Tris-HCl, pH 7.0, 1M NaCl, five times the volume of the column. The elution process was controlled by measuring optical density of the run-off solution at 260 nm. Chromatographic fractions containing the gene therapy DNA vector GDTT1.8NAS12-DDC were joined together and subjected to gel filtration by Superdex 200 sorbent (GE, USA). The column was equilibrated with phosphate buffered saline. The elution process was controlled by measuring optical density of the run-off solution at 260 nm, and the fractions were analysed by agarose gel electrophoresis. The fractions containing the gene therapy DNA vector GDTT1.8NAS12-DDC were joined together and stored at −20° C. To assess the process reproducibility, the indicated processing operations were repeated five times. All processing operations for the Escherichia coli strains JM110-NAS/GDTT1.8NAS12-IL10, or the Escherichia coli JM110-NAS/GDTT1.8NAS12-IL13, or the Escherichia coli JM110-NAS/GDTT1.8NAS12-IFNB1, or the Escherichia coli JM110-NAS/GDTT1.8NAS12-TNFRSF4, or the Escherichia coli JM110-NAS/GDTT1.8NAS12-TNFSF10, or the Escherichia coli JM110-NAS/GDTT1.8NAS12-BCL2, or the Escherichia coli JM110-NAS/GDTT1.8NAS12-HGF, or the Escherichia coli JM110-NAS/GDTT1.8NAS12-IL2 were performed in a similar way.

The process reproducibility and quantitative characteristics of final product yield confirm the producibility and constructability of the gene therapy DNA vector GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention is achieved, namely the construction of the gene therapy DNA vectors enhancing the expression level of DDC, IL10, IL13, IFNB1, TNFRSF4, TNFSF10, BCL2, HGF, and IL-2 genes that combine the following properties:
I) the effectiveness of increase of the expression of therapeutic genes in eukaryotic cells due to the obtained gene therapy vectors with a minimum length,
II) possibility of safe use in gene therapy of human beings and animals due to the absence of regulatory elements representing the nucleotide sequences of viral genomes and antibiotic resistance genes in the gene therapy DNA vector,
III) producibility and constructability in the strains on an industrial scale,
IV) as well as the purpose of the constructions of strains carrying these gene therapy DNA vectors for the production of these gene therapy DNA vectors is achieved.

This is supported by the following examples:
Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 for Clause I;
Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 for Clause II;
Examples 34, 35 for Clause III and Clause IV.

### INDUSTRIAL APPLICABILITY

All the examples above confirm industrial applicability of the proposed gene therapy DNA vector based on the gene therapy DNA vector GDTT1.8NAS12 carrying the therapeutic gene selected from the group of DDC, IL10, IL13, IFNB1, TNFRSF4, TNFSF10, BCL2, HGF, and IL-2 genes in order to increase the expression level of these therapeutic genes, the Escherichia coli strain JM110-NAS/GDTT1.8NAS12-DDC, or the Escherichia coli JM110-NAS/GDTT1.8NAS12-IL10, or the Escherichia coli JM110-NAS/GDTT1.8NAS12-IL13, or the Escherichia coli JM110-NAS/GDTT1.8NAS12-IFNB1, or the Escherichia coli JM110-NAS/GDTT1.8NAS12-TNFRSF4, or the Escherichia coli JM110-NAS/GDTT1.8NAS12-TNFSF10, or the Escherichia coli JM110-NAS/GDTT1.8NAS12-BCL2, or the Escherichia coli JM110-NAS/GDTT1.8NAS12-HGF, or the Escherichia coli JM110-NAS/GDTT1.8NAS12-IL2 carrying the gene therapy DNA vector, method of the gene therapy DNA vector production, method of the gene therapy DNA vector production on an industrial scale.

### List of Abbreviations

GDTT1.8NAS12—a gene therapy vector devoid of sequences of viral genomes and antibiotic resistance markers (vector therapeutic virus-antibiotic-free)
L-DOPA—1-3,4-dihydroxyphenylalanine
DNA—deoxyribonucleic acid
AADC—aromatic L-amino acid decarboxylase
cDNA—complementary deoxyribonucleic acid
RNA—ribonucleic acid
mRNA—messenger ribonucleic acid
bp—base pair
PCR—polymerase chain reaction
ml—millilitre, ul—microlitre
mm3—cubic millimetre
1—litre
ug—microgram
mg—milligram
g—gram
umol—micromol
mM—millimol
MPTP—1-methyl-4-phenyl-1,2,3,6-tetrahydropyridine
min—minute
s—second
rpm—rotations per minute
nm—nanometre
cm—centimetre
mW—milliwatt
RFU—relative fluorescence unit
PBS—phosphate buffered saline
PBMC—peripheral blood mononuclear cells
CNS—central nervous system
EEM—experimental encephalomyelitis.

1.-24. (canceled)

25. A gene therapy DNA vector based on a gene therapy DNA vector GDTT1.8NAS12 in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of a central nervous system, with disorders of oxygen supply to brain and spinal cord, with deviations in neuronal energy metabolism and axonal transport, or with autoimmune demyelinating processes, including multiple sclerosis, while the gene therapy DNA vector contains a coding region of the therapeutic gene selected from DDC, or IL10, or IL13, or IFNB1, or TNFRSF4, or TNFSF10, or BCL2, or HGF, or IL2, each cloned into the gene therapy DNA vector GDTT1.8NAS12 resulting in a gene therapy DNA vector GDTT1.8NAS12-DDC with a nucleotide sequence SEQ ID No. 1, or resulting in a gene therapy DNA vector GDTT1.8NAS12-IL10 with a nucleotide sequence SEQ ID No. 2, or resulting in a gene therapy DNA vector GDTT1.8NAS12-IL13 with a nucleotide sequence SEQ ID No. 3, or resulting in a gene therapy DNA vector GDTT1.8NAS12-IFNB1 with a nucleotide sequence SEQ ID No. 4, or resulting in a gene therapy DNA vector GDTT1.8NAS12-TNFRSF4 with a nucleotide sequence SEQ ID No. 5, or resulting in a gene therapy DNA vector GDTT1.8NAS12-TNFSF10 with a nucleotide sequence SEQ ID No. 6, or resulting in a gene therapy DNA vector GDTT1.8NAS12-BCL2 with a nucleotide sequence SEQ ID No. 7, or resulting in a gene therapy DNA vector GDTT1.8NAS12-HGF with a nucleotide sequence SEQ ID No. 8, or resulting in a gene therapy DNA vector GDTT1.8NAS12-IL2 with a nucleotide sequence SEQ ID No. 9.

26. The gene therapy DNA vector based on the gene therapy DNA vector GDTT1.8NAS12 containing the coding region of the therapeutic gene selected from DDC, or IL10, or IL13, or IFNB1, or TNFRSF4, or TNFSF10, or BCL2, or HGF, or IL2 as per claim 25, said DNA vector is unique due to a fact that each of the constructed gene therapy DNA vectors: GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 uses nucleotide sequences that don't constitute antibiotic resistance genes, viral genes, or elements of viral genomes, which ensures its safe use.

27. A method of gene therapy DNA vector production based on the gene therapy DNA vector GDTT1.8NAS12 carrying the therapeutic gene DDC, or IL10, or IL13, or IFNB1, or TNFRSF4, or TNFSF10, or BCL2, or HGF, or IL2 as per claim 25, that involves obtaining each of the gene therapy DNA vectors: GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2 as follows: the coding region of the therapeutic gene DDC, or IL10, or IL13, or IFNB1, or TNFRSF4, or TNFSF10, or BCL2, or HGF, or IL2 is cloned to DNA vector GDTT1.8NAS12, and the gene therapy DNA vector GDTT1.8NAS12-DDC, SEQ ID No. 1, or GDTT1.8NAS12-IL10, SEQ ID No. 2, or GDTT1.8NAS12-IL13, SEQ ID No. 3, or GDTT1.8NAS12-IFNB1, SEQ ID No. 4, or GDTT1.8NAS12-TNFRSF4, SEQ ID No. 5, or GDTT1.8NAS12-TNFSF10, SEQ ID No. 6, or GDTT1.8NAS12-BCL2, SEQ ID No. 7, or GDTT1.8NAS12-HGF, SEQ ID No. 8, or GDTT1.8NAS12-IL2, SEQ ID No. 9 respectively, is obtained, while the coding region of the DDC, or IL10, or IL13, or IFNB1, or TNFRSF4, or TNFSF10, or BCL2, or HGF, or IL2 therapeutic gene is obtained by isolating total RNA from the biological human tissue sample followed by the reverse transcription reaction and PCR amplification using the obtained oligonucleotides and cleaving the amplification product by corresponding restriction endonucleases, while cloning to the gene therapy DNA vector GDTT1.8NAS12 is performed by SalI and KpnI or BamHI and SalI restriction sites, while the selection is performed without antibiotics.

28. The method of use of gene therapy DNA vector based on gene therapy DNA vector GDTT1.8NAS12 carrying the therapeutic gene DDC, or IL10, or IL13, or IFNB1, or TNFRSF4, or TNFSF10, or BCL2, or HGF, or IL2 as per claim 25, namely GDTT1.8NAS12-DDC, or GDTT1.8NAS12-IL10, or GDTT1.8NAS12-IL13, or GDTT1.8NAS12-IFNB1, or GDTT1.8NAS12-TNFRSF4, or GDTT1.8NAS12-TNFSF10, or GDTT1.8NAS12-BCL2, or GDTT1.8NAS12-HGF, or GDTT1.8NAS12-IL2, in order to treat diseases associated with progressive pathological changes in neural tissue structure and neurons function, including death thereof, associated with genetic factors, including mutations of genes encoding proteins that are critical for normal neurons functioning, including Huntington's disease, hereditary forms of amyotrophic lateral sclerosis, and with impaired folding of tertiary structure of proteins, including Parkinson's disease, Alzheimer's disease, with traumatic injuries of the central nervous system, with disorders of oxygen supply to brain and spinal cord, with deviations in neuronal energy metabolism and axonal transport, or with autoimmune demyelinating processes, including multiple sclerosis that involves transfection of the cells of patient or animal organs and tissues with the selected gene therapy DNA vector carrying the respective therapeutic gene or several gene therapy DNA vectors carrying therapeutic genes based on gene therapy DNA vector GDTT1.8NAS12 from the group of constructed gene therapy DNA vectors, and/or injection of autologous cells of the patient or animal transfected with the selected gene therapy DNA vector or several gene therapy DNA vectors from the group of constructed gene therapy DNA vectors into the organs and tissues of the patient or animal, and/or the injection of the selected gene therapy DNA vector carrying the respective therapeutic gene or several selected gene therapy DNA vectors from the group of constructed gene therapy DNA vectors into the organs and tissues of the patient or animal, or a combination of the indicated methods.
